SEQUENCE LISTING (161 sequences)

SEQ ID NO: 1 - DNA, 1592 bp, Medicago truncatula
  source 1..1592: mol_type = genomic DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 2 - DNA, 2000 bp, Zea mays
  source 1..2000: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 3 - DNA, 4101 bp, synthetic construct
  misc_feature 1..4101: note = Cas9 nucleic acid sequence
  source 1..4101: mol_type = other DNA
gacaagaagtacagcatcgggctggcgatcgggaccaactccgtcggctgggctgtgattaccgacgagtacaaggtgccatccaagaagttcaaggtcctcggcaacactgaccggcacagcattaagaagaacctgattggggcgctgctgttcgattcgggggagactgcggaggcgaccaggctgaagcggactgcgcgccggaggtacaccaggaggaagaatcggatctgctacctccaggagattttctcgaatgagatggccaaggtggacgattccttcttccatcgcctggaggagtcgttcctcgttgaggaggacaagaagcatgagaggcatcccattttcgggaatatcgttgacgaggtggcttaccatgagaagtacccgaccatctaccatctgcggaagaagctcgtcgattcgaccgataaggccgacctgcggctgatctacctggccctcgcgcacatgattaagttccggggccatttcctcatcgagggcgacctcaacccggacaactcggacgtggataagctcttcattcagctcgtgcagacatacaaccagctcttcgaggagaatcccattaacgcctcgggggtcgacgctaaggctattctctcggctcggctgtcgaagtcgcgccggctggagaatctcattgcccagctcccaggcgagaagaagaacggcctcttcggcaacctgattgccctgtcgctggggctcacaccgaatttcaagtcgaacttcgacctcgccgaggacgctaagctccagctcagcaaggatacttacgatgatgacctcgataacctgctcgcccagattggggatcagtacgcggatctgttcctcgcggccaagaatctcagcgatgctattctcctgtcggacattctccgcgtcaacacagagattactaaggccccactgtcggcgagcatgattaagaggtacgatgagcatcatcaggacctgacactgctcaaggcgctggtccggcagcagctccccgagaagtacaaggagattttcttcgatcagtcaaagaatgggtacgcgggctacattgatggcggcgcgtcccaggaggagttctacaagttcattaagcccatcctggagaagatggacgggaccgaggagctgctggtgaagctcaatcgggaggacctgctccggaagcagcgcacattcgacaatggctcgattcctcaccagattcacctgggcgagctgcacgccattctccgcaggcaggaggacttctacccgttcctcaaggacaaccgcgagaagatcgagaagatcctgaccttccggattccatactacgtggggccgctcgcgcgggggaactcccggttcgcgtggatgactcgcaagtccgaagaaacgattacaccgtggaatttcgaggaggtcgtcgacaagggcgctagtgcgcagtcattcattgagaggatgaccaatttcgataagaacctgcctaacgagaaggtgctgccgaagcattcgctgctctacgagtacttcaccgtttacaatgagctgaccaaggtgaagtatgtgactgagggcatgaggaagccagcgttcctgagcggcgagcagaagaaggctatcgtggacctgctcttcaagactaaccggaaggtgactgtgaagcagctcaaggaggactacttcaagaagattgagtgcttcgattccgttgagattagcggggtggaggatcggttcaatgcttcgctcgggacataccacgatctcctgaagatcattaaggataaggacttcctcgacaacgaggagaacgaggacattctcgaagatattgtcctgaccctcaccctcttcgaggatcgggagatgatcgaggagaggctcaagacatacgctcatctgttcgatgataaggtcatgaagcagctgaagcgcaggcggtacacagggtgggggcggctgagccggaagctgatcaacgggattcgggataagcagtccgggaagacaattctcgacttcctcaagtccgacgggttcgctaaccggaacttcatgcagctcattcatgatgactcgctgacattcaaggaggatattcagaaggcgcaggtttcggggcagggcgactcgctccacgagcatattgcgaatctggcgggctcccccgcgattaagaagggcattctgcaaaccgtcaaggtggttgatgagctggtcaaggtcatggggcggcataagccagagaatattgtcatcgagatggcgcgggagaatcagaccacacagaaggggcagaagaactcacgggagcggatgaagcgcatcgaggagggcatcaaggagctggggtcgcagatcctgaaggagcatcccgtggagaacactcagctgcaaaatgagaagctgtacctctactacctccagaacgggagggacatgtatgtggatcaggagctggatattaataggctgagcgattacgatgtcgaccacattgtcccacagtcgttcctgaaggacgacagcattgacaacaaggtgctgacccgctcggataagaacaggggcaagagcgataatgttccaagcgaggaggttgtgaagaagatgaagaactactggcggcagctcctgaacgcgaagctcatcacacagcggaagttcgacaacctcaccaaggctgagcgcgggggcctgagcgagctggacaaggcggggttcattaagaggcagctggtcgagacacggcagattacaaagcatgttgcgcagattctcgattcccggatgaacaccaagtacgatgagaacgataagctgattcgggaggtcaaggtaattaccctgaagtccaagctggtgtccgacttcaggaaggacttccagttctacaaggttcgggagatcaacaactaccaccacgcgcatgatgcctacctcaacgcggtcgtggggaccgctctcatcaagaagtacccaaagctggagtcagagttcgtctacggggattacaaggtttacgacgtgcggaagatgatcgctaagagcgagcaggagattggcaaggctaccgctaagtacttcttctactccaacatcatgaacttcttcaagacagagattaccctcgcgaatggcgagatccggaagaggcccctcatcgagacaaatggggagacaggggagattgtctgggataaggggcgggatttcgcgaccgtccggaaggtcctgtcgatgccccaggttaatattgtcaagaagactgaggtccagactggcggcttctcaaaggagtcgattctcccaaagaggaactccgataagctcattgctcggaagaaggattgggaccccaagaagtacgggggattcgactcccccactgttgcttactctgttctggttgttgctaaggtggagaaggggaagtcgaagaagctgaagagcgtgaaggagctgctcgggattacaattatggagaggtcatccttcgagaagaatcccatcgacttcctggaggccaagggctacaaggaggtgaagaaggacctgattattaagctgcccaagtactcgctcttcgagctggagaatgggcggaagcggatgctggcgtccgcgggggagctgcaaaaggggaacgagctggcgctcccctccaagtatgtgaacttcctctacctggcgtcgcactacgagaagctgaaggggtccccagaggataatgagcagaagcagctcttcgtcgagcagcataagcactacctggacgagattatcgagcagattagcgagttctcgaagcgggtcatcctcgcggatgcgaacctggataaggtgctcagcgcctacaataagcaccgggacaagccgattcgggagcaggcggagaatattattcacctcttcacactcaccaacctcggggcaccagctgcgttcaagtacttcgacactactatcgaccggaagcggtacacctcgacgaaggaggtgctcgacgccaccctcattcaccagtcgatcacaggcctgtacgagacacggattgacctgtcccagctcgggggcgac

SEQ ID NO: 4 - DNA, 4101 bp, synthetic construct
  misc_feature 1..4101: note = Cas9 nucleic acid sequence
  source 1..4101: mol_type = other DNA
gacaagaagtactccattggcctggcgattgggacaaactcggtggggtgggccgtgattacggatgagtacaaggttccaagcaagaagttcaaggtcctcgggaacacagatcggcattcgattaagaagaatctcattggggcgctcctcttcgactcgggggagacagcggaggctaccaggctcaagcggacagccaggcggcggtacacaaggcggaagaatcgcatctgctacctccaggagattttctcgaatgagatggcgaaggtggacgacagcttcttccatcggctggaggagtccttcctggtggaggaggataagaagcacgagaggcatccaattttcgggaacatcgtggacgaggttgcgtaccatgagaagtaccctacaatctaccatctgcggaagaagctggttgactccacagacaaggcggacctgaggctgatctacctcgctctggcccacatgattaagttccgcgggcatttcctgatcgagggggacctgaatcccgacaattcggatgtggacaagctcttcatccagctggtgcagacctacaaccagctgttcgaggagaatcccatcaatgcgtcgggcgttgacgctaaggccattctgtccgctaggctgtcgaagagcaggaggctggagaacctgatcgcccagctgccaggcgagaagaagaatgggctcttcgggaatctgattgcgctctccctggggctgacaccgaacttcaagagcaatttcgatctggctgaggacgcgaagctccagctctcgaaggacacttacgacgatgacctcgataacctcctcgcgcagatcggggaccagtacgctgatctcttcctcgccgctaagaacctctcggatgctatcctgctctccgacattctccgggttaataccgagattacaaaggccccactgtcggcgtccatgatcaagcggtacgatgagcatcatcaggatctcaccctgctcaaggccctcgtgcggcagcagctgcccgagaagtacaaggagattttcttcgaccagagcaagaatgggtacgctggctacattgacggcggggcctcacaggaggagttctacaagttcatcaagccaatcctggagaagatggatgggacagaggagctgctggtgaagctcaaccgggaggatctgctcaggaagcagcggacgttcgacaacgggtcgattccccatcagatccacctgggggagctgcacgcgatcctgcgccggcaggaggatttctaccctttcctgaaggataatcgggagaagatcgagaagattctcaccttccggattccctactacgtcgggccactcgcgcggggcaatagcaggttcgcctggatgacacggaagagcgaggagacaatcaccccctggaacttcgaggaggttgtcgacaagggggcgtccgcccagtcattcattgagcggatgaccaatttcgacaagaatctgccaaatgagaaggttctcccaaagcatagcctcctctacgagtacttcactgtttacaacgagctgaccaaggtgaagtatgtgaccgagggcatgcggaagcccgcgttcctgtccggcgagcagaagaaggccattgtggacctcctgttcaagaccaatcgcaaggtcacagtcaagcagctcaaggaggattacttcaagaagatcgagtgcttcgactcggttgagattagcggggtggaggatcggttcaacgcgagcctcggcacttaccacgacctcctgaagatcatcaaggataaggacttcctcgacaacgaggagaacgaggatattctggaggacatcgtgctcaccctgacgctgttcgaggatcgggagatgatcgaggagcgcctgaagacctacgctcatctcttcgatgataaggtcatgaagcagctgaagaggaggcggtacaccgggtggggccgcctgagcaggaagctcattaacgggatcagggacaagcagagcggcaagaccatcctggacttcctcaagagcgatggcttcgccaaccggaatttcatgcagctcatccacgacgactccctcaccttcaaggaggacattcagaaggctcaggtcagcggccagggcgactcgctgcatgagcacatcgctaacctggcgggcagcccagccatcaagaagggcatcctccagacagtgaaggtcgtggatgagctggtgaaggtcatgggccggcataagcccgagaatattgtgattgagatggcgcgggagaatcagaccactcagaagggccagaagaactcgcgggagcgcatgaagaggatcgaggaggggattaaggagctgggcagccagattctcaaggagcaccccgtggagaatacccagctccagaacgagaagctgtacctctactacctccagaatgggcgggacatgtatgttgatcaggagctggacatcaatcgcctctcggattacgacgtggaccacatcgtgccccagagcttcctgaaggatgatagcatcgacaataaggtcctgacccgctccgacaagaatcgcggcaagagcgacaacgtgccgagcgaggaggtcgtgaagaagatgaagaactactggcggcagctgctgaacgcgaagctcattacacagcggaagttcgataacctgacgaaggcggagaggggcggcctctccgagctggacaaggcgggcttcattaagaggcagctcgtggagactcgccagatcaccaagcacgtggctcagatcctcgatagccggatgaatacgaagtacgatgagaatgacaagctcatccgggaggtgaaggtaatcaccctgaagtcaaagctcgttagcgatttccggaaggacttccagttctacaaggtgcgggagattaacaactaccatcatgcgcacgatgcgtacctcaatgcggtggtgggcacagccctgattaagaagtaccccaagctggagagcgagttcgtctacggggactacaaggtgtacgatgttcggaagatgatcgccaagagcgagcaggagattgggaaggccaccgctaagtacttcttctactcgaatattatgaatttcttcaagaccgagatcacactcgctaatggggagattcggaagcggcccctcatcgagactaacggggagactggcgagattgtgtgggacaaggggcgcgacttcgctaccgtgcgcaaggtcctctcgatgccccaggttaatattgttaagaagacagaggtgcagacgggcgggttctccaaggagtctatcctgccgaagcggaactcggacaagctgatcgcccgcaagaaggattgggaccccaagaagtacgggggattcgatagcccaaccgtggcttacagcgtcctggtggtcgccaaggttgagaaggggaagtcgaagaagctcaagagcgttaaggagctgctgggcatcaccatcatggagcggtccagcttcgagaagaatcctatcgacttcctggaggctaaggggtacaaggaggtcaagaaggacctgatcattaagctgcccaagtactctctgttcgagctggagaacgggaggaagcggatgctggcgtctgctggcgagctacagaagggcaatgagctggcgctcccctcgaagtatgtcaacttcctctacctggcttcccattacgagaagctgaagggctcgcccgaggataatgagcagaagcagctcttcgtggagcagcacaagcactacctcgacgagatcattgagcagatttcggagttctcgaagcgggtcattctcgcggacgcgaacctcgacaaggtcctctcggcgtacaacaagcaccgggacaagcccatccgggagcaggccgagaacattatccacctcttcacactgaccaacctcggcgctcccgccgcgttcaagtacttcgacaccaccattgaccgcaagagatacacatccaccaaggaggtgctggacgcgaccctcatccaccagagcatcacaggcctctacgagacacggatcgacctctcgcagctcgggggcgat

SEQ ID NO: 5 - DNA, 4092 bp, synthetic construct
  misc_feature 1..4092: note = Cas9 nucleic acid sequence
  source 1..4092: mol_type = other DNA
gacaagaagtactcgatcggcctggcgattggcacaaacagcgtggggtgggctgtgatcactgatgagtacaaggtgccatcgaagaagttcaaggtgctggggaatacagaccggcattcgatcaagaagaatctcattggcgctctcctcttcgattccggcgagactgctgaggcgacccgcctgaagcgcaccgcccggcggcgctacactcggcggaagaataggatttgctacctccaggagattttctcgaatgagatggccaaggtggatgacagcttcttccaccgcctggaggagtcgttcctggtcgaggaggacaagaagcatgagcggcaccctatcttcgggaatatcgttgatgaggtcgcctaccacgagaagtaccccactatctaccatctccgcaagaagctcgtggacagcacagataaggccgacctccgcctgatctacctcgccctcgcgcacatgattaagttccgggggcacttcctcattgagggggatctgaatcccgataactccgacgtggacaagctgttcatccagctggtgcagacatacaaccagctgttcgaggagaatcccatcaacgcgagcggcgtggacgctaaggccattctgtcggctaggctctcgaagtcgaggcggctggagaacctgattgcgcagctccccggcgagaagaagaacgggctgttcgggaatctcatcgccctctccctcggcctcacaccaaacttcaagagcaatttcgacctggctgaggacgctaagctgcaactctcaaaggatacatacgatgacgacctggacaatctcctggctcagatcggcgaccagtacgctgacctgttcctcgcggccaagaatctgtcggacgcgattctcctcagcgacatcctgcgcgtcaataccgagattacgaaggctccactgtctgcgtcaatgattaagcggtacgatgagcatcaccaggatctgaccctcctgaaggcgctcgtgcggcagcagctgcccgagaagtacaaggagattttcttcgatcagagcaagaatggctacgccggctacatcgacgggggcgcgagccaggaggagttctacaagttcatcaagcccatcctggagaagatggacggcaccgaggagctactcgtgaagctcaatcgggaggatctcctccggaagcagcggacattcgataacgggtctatcccacaccagatccacctcggcgagctgcatgcgattctgcggcggcaggaggatttctaccctttcctgaaggacaaccgggagaagatcgagaagatcctcacattccggattccatactacgtcggccccctggcgaggggcaatagccggttcgcgtggatgacaaggaagtccgaggagactattaccccgtggaatttcgaggaggtggttgacaagggcgcttccgcgcagagcttcattgagcggatgacaaacttcgacaagaatctccccaacgagaaggtcctgccgaagcatagcctcctgtacgagtacttcaccgtctacaatgagctaactaaggtcaagtatgtgacagagggcatgaggaagccagccttcctctcaggcgagcagaagaaggccattgtggacctcctgttcaagacaaaccgcaaggtgacagtgaagcagctgaaggaggattacttcaagaagattgagtgcttcgactcagtggagatttcaggcgtggaggatcggttcaacgcgagcctggggacttaccacgacctgctgaagattattaaggacaaggacttcctggataacgaggagaatgaggacatcctggaggatattgtgctcaccctcaccctgttcgaggacagggagatgattgaggagaggctcaagacctacgcgcacctgttcgatgacaaggtcatgaagcagctgaagaggcggcgctacactgggtggggccgcctgtcgcggaagctgatcaacggcattcgggataagcagtccgggaagaccattctggatttcctgaagtcggacggcttcgccaacaggaatttcatgcagctgatccacgacgactccctcaccttcaaggaggacattcagaaggcccaggttagcggccagggggactcactccacgagcatattgccaatctggccggctctccagctatcaagaagggcatcctgcaaacagttaaggttgttgacgagctggttaaggtcatggggcggcataagcccgagaacattgtcatcgagatggctcgggagaaccagacaactcagaagggccagaagaactccagggagcgcatgaagcggattgaggagggcattaaggagctggggtcccagatcctcaaggagcaccctgtcgagaacactcagctgcaaaacgagaagctctacctgtactacctccagaacgggcgggatatgtatgtggatcaggagctggacatcaacaggctctccgactacgacgtggatcacattgtcccacagtctttcctcaaggatgattccatcgacaacaaggtgctgacgcgcagcgacaagaatagggggaagtcggacaacgttccgagcgaggaggtcgtgaagaagatgaagaattactggaggcagctcctgaatgcgaagctgatcactcagaggaagttcgacaatctgacaaaggcggagaggggcgggctctcggagctggataaggcgggcttcatcaagcggcagctcgttgaaacccggcagatcaccaagcatgtcgcccagatcctcgatagccgcatgaacaccaagtacgatgagaacgacaagctcattcgggaggttaaggtcattacgctgaagtccaagctcgtcagcgacttcaggaaggatttccagttctacaaggttcgggagattaacaactaccaccacgcgcatgatgcgtacctgaacgctgttgtcggcactgctctcatcaagaagtacccaaagctggagtccgagttcgtctacggggactacaaggtctacgatgtccggaagatgatcgccaagtcggagcaggagatcgggaaggctactgcgaagtacttcttctacagcaacattatgaatttcttcaagacggagattacgctggcgaacggggagattaggaagaggcccctcattgagactaatggggagacaggcgagattgtttgggacaagggccgcgacttcgcgactgtgcggaaggtcctgtccatgccacaggtgaatattgttaagaagacagaggtgcagactgggggcttctcgaaggagagcattctcccaaagcggaacagcgataagctcatcgcgcgcaagaaggattgggaccctaagaagtacggcggcttcgattctcccactgtggcctactccgttctcgtggttgccaaggttgagaaggggaagtcgaagaagctgaagtcggtcaaggagctgctcgggattacaatcatggagcggagcagcttcgagaagaaccctattgatttcctggaggccaagggctacaaggaggttaagaaggatctcattatcaagctccctaagtactctctgttcgagctggagaatggccggaagaggatgctggcctcggctggcgagctacagaaggggaatgagctggccctcccgtcgaagtatgtgaatttcctgtacctcgcgtcgcactacgagaagctcaagggcagcccggaggataatgagcagaagcagctcttcgtggagcagcataagcactacctggacgagatcattgagcagatcagcgagttctcgaagcgggttattctggctgatgctaacctggacaaggttctgagcgcctacaataagcatcgcgacaagccgattcgcgagcaggcggagaatattatccacctgttcaccctcactaacctcggggctcccgcggccttcaagtacttcgataccacaatagataggaagcggtacacctcgacgaaggaggtcctcgacgccacactcatccatcagtcgattacaggcctgtacgagacacggattgacctctcgcagctg

SEQ ID NO: 6 - DNA, 4101 bp, synthetic construct
  misc_feature 1..4101: note = Cas9 nucleic acid sequence
  source 1..4101: mol_type = other DNA
gacaagaagtattccataggcctggctatcggcaccaacagcgtgggctgggccgtcatcaccgacgagtacaaagtgccgagtaaaaagttcaaagtgctcggcaacaccgaccgccactccataaagaaaaacctgatcggggcgctcctgttcgacagcggcgagacggcggaggccacccgcttgaaacgcacggcccgacggcgctacacgcggcgcaagaaccggatctgttacctacaggagattttctctaacgagatggcgaaggtggacgactcgttctttcaccgcctcgaagagtccttcctcgtggaggaggacaagaaacacgagcgccacccgatcttcggcaacatcgtggacgaggtggcctaccacgagaagtacccgaccatctaccacctccggaagaaactcgtggacagcacggacaaggccgacctgaggctcatctacctcgccctggcgcacatgattaagttccggggccacttcctgatcgagggcgacctgaacccggacaacagcgacgtggacaagctgttcatccagctagtccagacctacaaccagcttttcgaggaaaaccccatcaacgccagcggggtggacgcgaaggcgatcctgtccgcccggctgagcaagtcccggcggctggagaacctcatcgcgcagttgcccggcgagaagaagaacgggctgttcgggaacctgatcgccctctccctggggctcaccccgaacttcaagtccaacttcgacctcgccgaggacgccaaactacagctgagcaaggacacctacgacgacgacctcgacaacctgctggcccagatcggggaccagtacgcagacctgttcctcgccgccaagaacctctccgacgccatcctgctgtcggacatcctgcgggtgaacacggagatcacgaaggccccgctctcggcctcgatgattaaacgctacgacgagcaccaccaggacttgaccctcctcaaggcgctggtccgccagcagcttcccgagaagtacaaggaaatctttttcgatcagagcaagaacgggtacgccgggtacatcgacggcggggcgtcccaggaggagttctacaagttcatcaagcccatcctggagaaaatggacgggaccgaggagctgctcgtgaagctcaaccgcgaagatttgctccgcaagcagcgcacgttcgacaacgggtcgatcccgcaccagatccacctgggcgagctgcacgcgatcctcaggcgtcaggaagacttctaccccttcctcaaggacaaccgcgagaagatagagaagattctgaccttcagaattccttattacgtgggcccgctggctcggggcaactcgcgcttcgcctggatgacgcgcaagtccgaggagaccatcaccccgtggaacttcgaggaggtggtggataagggtgcctcggcccagtccttcatcgagcggatgaccaacttcgacaagaacctgccgaacgagaaggtgctccccaagcacagcctgctctacgaatatttcacggtgtacaacgagctgacgaaggtcaagtacgtgaccgagggaatgaggaaacctgcattcctctccggggagcagaagaaagccatagtcgacctcctgttcaagaccaaccggaaggtcaccgtcaagcagctcaaggaggactacttcaagaagatcgagtgcttcgattcagtggagatcagcggcgtcgaggaccggttcaacgccagcctgggcacctaccacgacctgctcaagatcatcaaggacaaggacttcctcgacaacgaggagaacgaggacatcctggaggacatcgtgctgaccctgacgctcttcgaggaccgcgagatgatcgaggagcgcctcaagacctacgcccacctgttcgacgacaaggtgatgaagcagctcaagcggcggagatatactgggtggggccgcctctcccggaagctcattaacggtatcagggataagcagtccgggaagacgatcctcgacttcctcaagtcggacgggttcgccaaccgcaacttcatgcagctcatccacgacgactccctgacgttcaaggaggacatccagaaggcccaagtgtctggtcaaggtgactcgctccacgagcacatcgccaacctcgcgggcagcccggccatcaagaagggaatactccagaccgtcaaggtggtggacgagctggtgaaggtcatgggccgccacaagccggagaacatcgtcatcgagatggcgcgggagaaccagaccacgcagaaggggcagaaaaatagccgtgagcgcatgaagcgcatcgaggaggggattaaggagttgggcagccagatcctcaaggagcaccctgtggagaacacgcagttgcaaaacgagaagctctacctgtactacctccagaacgggagggatatgtacgtggaccaagaactggacatcaaccgcctgtccgactacgacgtggaccacatcgtgccgcagagcttcctcaaggacgacagcatcgacaacaaggtgctcacccggtccgacaagaatcggggcaagtccgacaacgtgcccagcgaggaggtcgtcaaaaagatgaaaaactactggcgacaactactgaacgccaagctcatcacccagcgcaagttcgacaacctcacaaaagccgagcgcggcgggttgagcgagctggacaaggccgggttcatcaagcgccagctcgtcgagacgcgccagatcacgaagcacgtcgcgcagatactcgacagccggatgaacaccaagtacgacgagaacgacaagctcatccgggaggtgaaggtcatcaccctcaagtcgaagctcgtgagcgacttccgcaaggacttccagttctacaaggtccgggagatcaacaactaccaccacgcccacgatgcttatcttaacgccgtggtggggacggccctcattaagaaatacccgaagctggagtcggagttcgtgtacggcgactacaaggtgtacgacgtcaggaagatgatcgccaagtccgaacaggagatcgggaaggccacggcgaaatacttcttctacagcaacatcatgaacttcttcaagaccgagatcaccctcgccaacggcgagatccgcaagcgcccgctcatcgagacgaacggggagaccggcgagatcgtctgggacaaggggcgcgacttcgccactgtgcggaaggtgctgtcgatgccccaggtcaacatcgtcaagaagacggaggtccagacgggcgggttcagcaaggagagcatcctgccgaagcgcaacagcgacaagctgatcgcccgcaaaaaggactgggatccaaaaaagtacggcggcttcgacagccccaccgtcgcctacagcgtcctcgtcgtcgctaaagtcgagaagggcaagtccaaaaagctcaagagcgtcaaggagctgctcgggatcaccatcatggagcggtccagcttcgagaagaacccaattgatttcctggaggcgaagggctacaaggaggtcaagaaagacctcatcataaagctgccgaagtactcactcttcgagctggagaacgggcgcaagcggatgctggcgtcggccggagagctccaaaagggcaacgagctggcgctgccgagcaagtacgtgaacttcctctacctggcgtcccactacgagaagctcaagggcagtccagaggataacgagcagaagcagctattcgtggagcagcacaagcactacctggacgagatcatcgagcagatcagcgagttctccaagcgcgtcatcctggcggacgccaacctggacaaggtgctgtccgcgtacaacaagcaccgcgacaagccgatccgcgagcaagccgagaacatcatccacctgttcaccctcacgaacctcggggcacccgccgccttcaaatatttcgacacgaccatcgaccgcaagcgctacaccagcacgaaggaggtgctcgacgccaccctgatccaccagagcatcaccgggctgtacgagacccgcatcgacctctcgcagctcggcggggac

SEQ ID NO: 7 - DNA, 4101 bp, synthetic construct
  misc_feature 1..4101: note = Cas9 nucleic acid sequence
  source 1..4101: mol_type = other DNA
gacaagaagtacagtattggattggccatcgggacgaacagcgtgggctgggccgtcatcaccgacgagtacaaggtgccatccaagaagtttaaggttctggggaataccgaccgccactcgatcaagaaaaatctcatcggggcgctgcttttcgacagcggcgagacggcggaagcgacgcggctcaagcggacggctcgtcgccgttacacccggcgtaagaaccgcatctgttacctccaggagatattcagcaacgagatggcgaaggtggacgactcctttttccaccgtcttgaggagtccttcctggtcgaggaggacaagaagcacgagcgccacccgatcttcgggaacatcgtggacgaggtggcctaccacgagaagtaccccacgatctaccacctccgcaaaaaactcgtggactcaactgacaaggccgatttgaggcttatctacctcgccctcgcccacatgattaagttccgtgggcacttcctaatcgagggtgacctcaaccccgacaactctgacgtggacaagctgttcatccagcttgtgcagacctacaatcagctctttgaggagaatccgatcaacgcatctggtgtggacgcaaaggccatcctcagcgcgcggctgagcaagtctaggcggttggagaacctgatcgcccaactgcccggcgagaagaaaaatggcctcttcggcaacctgatcgccctgtcgctggggctcacgccgaacttcaagagtaactttgacctggcggaggacgctaagctccagctatctaaggacacatacgacgacgacctggacaacctgctggcccagatcggcgaccagtacgccgacctcttcctagccgccaagaacctgtccgacgccatcctcctcagcgacatcctgcgcgtgaacacggagatcacgaaggctccgctcagcgcctccatgattaagcggtacgacgagcaccaccaagacctaactttactcaaagccctcgtgcggcagcagcttcccgagaagtacaaagagatattttttgatcagtccaagaacggttatgcgggctacatcgacggcggcgcgagccaggaggagttctacaagttcatcaagcccatcctggagaagatggacggcacggaggagctgctcgtgaagctcaaccgtgaagacctcctgcgaaagcagcgaaccttcgacaacggttcgatcccgcaccagatccacctcggggagctgcacgccatcctgaggcgacaggaggacttctaccctttcctaaaggacaaccgcgagaagattgaaaaaatcctgacgtttcgcataccctactacgtcggcccgctggcgcgcggcaactcccggttcgcctggatgacccgtaagagcgaggagacgatcaccccgtggaacttcgaggaggtcgtggacaagggcgcgagcgcgcagagcttcatcgagcgcatgaccaacttcgacaagaacctcccgaacgagaaggtgctcccaaagcactccctcctgtacgagtatttcaccgtgtacaacgagttgacaaaggtgaagtacgtgacggagggaatgcggaagcctgcgttcctctcgggcgagcagaagaaggcaatcgtggacctgctcttcaagaccaaccggaaggtgacggtgaagcagctcaaggaggactacttcaaaaaaatcgagtgcttcgactccgtggagataagcggcgtggaggaccgattcaacgcctccctcggcacctaccacgacctccttaagatcatcaaggacaaggacttcctggacaacgaggagaacgaggacatcctggaggacatcgtgctcaccctgaccctcttcgaggaccgggagatgatcgaggagcgcctcaagacgtacgcccacttgttcgacgacaaggtgatgaagcagctcaagcggcggcgatacaccgggtggggccgcctatcccgcaaacttatcaacggcatccgcgacaagcagtccggcaagacgatcctggatttcctcaagtcggacgggttcgccaaccggaacttcatgcagctcatccacgacgacagcctcacgttcaaggaggacatccagaaggcccaagtgagcggtcaaggggacagcctccacgagcacattgcgaaccttgctgggagccctgcgatcaagaaggggatattgcaaaccgtgaaggtcgtggacgagttggtgaaggtcatggggcgacacaagcccgagaacatcgtgatcgagatggccagggaaaatcagaccacgcagaagggccaaaaaaacagccgcgagcggatgaagcggatcgaggagggcatcaaggagctggggtcgcagatcctcaaggagcacccggtggagaacacgcagctccagaacgagaagctgtacctctattacctacagaacgggcgggatatgtacgtggaccaggagctagacatcaaccgcctgtccgactacgacgtggaccatatcgtcccgcagtcgttcttgaaggacgacagcatcgacaacaaggtgctcacaagatcggataagaatcgaggcaagtccgacaacgtgccctcggaggaggtggtcaagaaaatgaaaaactactggcggcagttgctgaacgccaagctcattacgcagcggaagttcgacaacctgacgaaggctgaacgtggtgggctcagcgagctagacaaggcggggttcatcaagcggcagctcgtcgagacccggcagatcaccaagcacgtggcgcagatcctggactcgcgcatgaacaccaagtacgacgagaacgacaagctcatccgtgaggtgaaggtcatcacccttaagtctaagctggtcagtgacttccgcaaggacttccagttctacaaggtccgggagatcaacaactaccaccacgcgcacgacgcctacctcaacgcggtggtggggacggcgcttattaagaaatatcccaagctggaaagcgagttcgtttacggcgactacaaggtgtacgacgtccgcaagatgatcgcaaagtcggaacaggaaatcggaaaggcgacggccaaatatttcttttactccaacatcatgaatttttttaagacggagatcaccctggcgaacggggagatccgcaagcggcccctcatcgagaccaacggggagacgggcgagatcgtctgggacaagggccgggacttcgccaccgtgcggaaggtgctttctatgcctcaagtcaatatcgtcaaaaagacagaggtgcagaccggcgggttcagcaaggagtctatcctgccgaagcgcaactcggacaagctcatcgcgcgcaagaaagactgggaccccaaaaaatatggcgggttcgactcgccgaccgtcgcctacagcgtcctcgtggtggctaaggtcgagaagggcaagagcaaaaagctaaagtcggtgaaggagctgctgggcatcaccatcatggagcgctcgtctttcgagaagaatccaatcgacttcctagaggcgaaggggtacaaggaggtcaaaaaggatcttatcatcaaactgccgaagtacagtctgttcgagctggagaacgggcggaagcggatgctggctagtgcgggcgagttgcagaagggcaacgagttggcactgccctccaagtacgtgaacttcctgtacctggcctcccactacgagaagctcaaggggagccccgaggacaacgagcagaagcagctattcgtcgagcagcacaagcactacctggacgagatcatcgagcagatcagtgagttctccaagcgggtcatcctcgcggacgccaacctggacaaggtgctgagcgcgtacaacaagcacagggacaagccaatcagggaacaggccgagaacatcatccacctgttcaccctgaccaacctgggtgcaccggctgccttcaagtactttgacacgaccatcgaccggaagcgctacacctccacgaaggaggtgctggacgccacgctgatccaccagagcatcaccgggctctacgagacacggatcgacctgagccagcttggcggggac

SEQ ID NO: 8 - DNA, 4092 bp, synthetic construct
  misc_feature 1..4092: note = Cas9 nucleic acid sequence
  source 1..4092: mol_type = other DNA
gacaaaaagtattccattggactcgctatcggcacgaacagcgtcgggtgggcggtcatcactgacgagtacaaggtgccgagcaagaagtttaaggtgctgggaaacaccgacaggcactcgatcaagaaaaatcttatcggggccctactcttcgactccggagaaaccgccgaggccacccggttgaagcgcacggcccgccgtcgctacaccaggcgcaagaaccggatctgctacctccaggagatattcagcaatgagatggcgaaggtggacgactcgttttttcacaggctagaggagtctttcctcgtggaggaggacaagaaacacgagcgccaccccatcttcggcaacatcgtggatgaggtggcatatcacgagaagtacccaaccatctaccacctccgcaaaaagctcgtggactctaccgacaaggccgacctccgtctgatctacctcgcgctggcccacatgattaagttccgaggacactttctgatcgagggcgacctgaacccagacaacagcgacgtggacaagctgttcatccaacttgtccagacctacaatcagctcttcgaggagaaccctatcaacgcctcgggcgtggacgcgaaggccatcctgtccgcccgcctgagcaagtcgcggcggctggagaacctgatcgcccagctccccggcgaaaaaaagaacggcctcttcggcaacctcatcgcgttgtcgctggggctcaccccgaacttcaagtccaacttcgacctggccgaggacgctaaactccagctctcgaaggatacctacgacgacgacctcgacaacctgctggcccagatcggcgaccagtacgcggaccttttcctggcggccaagaacctgagcgacgcgatcctccttagcgacatactccgtgtgaacaccgagatcacgaaggccccgctctccgcgtccatgattaagcgctacgacgagcaccaccaagaccttaccctgcttaaggcgctggtcaggcagcagttaccggagaagtacaaggagatcttttttgatcaatctaagaacggttacgccgggtacatcgacggcggcgcgtcccaggaggagttctacaagttcatcaagccgatcttggagaaaatggacgggaccgaggagctgctcgtgaagctcaaccgcgaagacctcctccgcaagcagcgcaccttcgacaacgggagcatcccgcaccagatccacctgggagagctgcacgcgatcctgcggagacaagaggacttctaccccttcctcaaggacaaccgggagaagattgaaaaaatacttacttttcgtatcccgtactacgtcgggccccttgcgaggggcaactccagattcgcgtggatgacccgcaagtccgaggagaccatcaccccgtggaacttcgaggaggtggtggacaagggcgcgtcggcccagtcgttcatcgagcgcatgaccaacttcgacaagaaccttccgaacgagaaggtgctcccgaagcacagcctgctctacgaatattttactgtgtacaacgagctgacgaaggtcaagtacgttacggaggggatgaggaagcccgccttcctctccggcgagcagaagaaagccattgtggatctcctgttcaagaccaaccgcaaggtgacggtgaaacagctcaaagaggactacttcaagaagatcgagtgcttcgactccgtagagatcagcggggtcgaggaccgcttcaacgcctcgctgggcacgtaccacgacctgctaaagattatcaaggacaaagacttcctagacaatgaggagaacgaggacattctggaggacatcgtgctgactctgacgctgttcgaagaccgcgagatgatcgaggagcggcttaagacgtacgcccacctgttcgacgacaaggtgatgaagcagttgaaacggcggcgctacaccgggtggggccgcctctcccgcaagctcatcaacggcatccgcgacaagcagtcggggaagacgatcctggacttcctcaagagcgacggcttcgccaaccgaaacttcatgcagctaatccacgacgacagcctgacgttcaaggaggacatccagaaggcccaagtgagcggccagggagactcgctacacgagcatatcgccaacctggctggcagcccggcgattaagaaaggaatcctccaaaccgtcaaagtggtggacgagctggtgaaggtgatgggccgccacaagcccgagaacattgtgatcgagatggcgcgggagaaccagacgacgcagaagggccaaaaaaatagcagggaaaggatgaagcgaatagaggaggggatcaaggagctggggagccagattctcaaagagcacccggtcgagaacacacagctccagaacgagaagctgtacctctactacctccaaaacggccgcgatatgtacgtggaccaggaactagacatcaaccggctgagcgactatgacgtggaccacatcgtgccgcagtccttcctcaaggacgactcgattgacaacaaagtgctcactagatccgacaagaacagaggcaagagcgataacgtcccgtcggaggaggtcgtcaagaaaatgaaaaactactggcggcagctcctaaacgccaagctcatcacgcagcgtaagttcgacaacctgacgaaggcggagcggggcgggctgagcgagctggacaaagcggggttcatcaagcggcagctcgttgagacgcggcagatcacaaagcacgtcgcgcaaatcctcgactcccgcatgaacaccaagtacgacgagaacgacaagctcatccgggaggtgaaggtcattacccttaaatcgaagctcgtcagcgactttcgtaaggacttccagttctacaaggtcagagagatcaacaactaccaccacgcccacgacgcctatctgaacgccgtggtgggcaccgcgcttattaagaagtaccccaagctggagtccgagttcgtgtacggcgactacaaggtttatgacgtcaggaagatgatcgccaagtcggaacaggagatcggaaaagctaccgccaaatatttcttctatagcaacatcatgaacttcttcaaaaccgagatcaccctcgccaacggcgagatccggaagcgcccgctcatcgagaccaacggggagaccggggagatcgtctgggacaaggggcgggacttcgctactgtccgaaaggtgctctccatgccacaagtgaatatcgtcaagaaaacagaggtgcagaccggagggttcagtaaggagtccatcctgcccaagcggaactccgacaagctaattgctcgcaaaaaggattgggatcctaaaaaatatggcggcttcgactcgcccacggtcgcctactctgtgctggtcgtggcgaaggtggagaagggcaagtccaagaagctcaagagcgtcaaggagctgctggggatcacgatcatggagcgtagttcgtttgagaagaatcccatcgacttcctggaggctaagggctacaaggaggtcaaaaaggacctcatcattaagctgccgaagtacagcctcttcgagctggagaacgggcggaagcgtatgctcgcctccgctggggagttacaaaaggggaacgagctggcgctgccgtctaagtacgtcaacttcctgtacctggcctcccactacgagaagctcaaggggtcgccggaggacaacgagcagaagcagctcttcgtagagcagcacaagcactacctggacgagatcatcgagcagatttcagagttctcaaagcgggtcatcctcgccgacgccaacctggacaaggtgctctcggcctacaacaagcaccgggacaagccgatccgcgaacaggccgaaaacatcatccacctgttcacgctcaccaacctcggtgccccggcggccttcaagtactttgacacgaccatcgaccggaagcgctatacctcgacgaaggaggtgctggacgccaccctgatccaccagtccatcaccgggctttacgagacccggatcgacctctcgcagcta

SEQ ID NO: 9 - DNA, 4101 bp, synthetic construct
  misc_feature 1..4101: note = Cas9 nucleic acid sequence
  source 1..4101: mol_type = other DNA
gacaagaagtatagtattggactcgccatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgaccacatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgat

SEQ ID NO: 10 - DNA, 4101 bp, synthetic construct
  misc_feature 1..4101: note = Cas9 nucleic acid sequence
  source 1..4101: mol_type = other DNA
gacaaaaagtattccatcgggcttgctatcggaaccaactctgtggggtgggcagttattaccgacgaatacaaggtgcccagcaagaagtttaaggttctggggaacacagatagacatagcataaagaaaaacctgataggcgcactgttgttcgactccggggaaacagccgaagctaccaggctgaagagaactgcaagaagaaggtacaccagaagaaaaaacagaatatgttatctccaagagattttctctaacgagatggccaaggtggacgactcattctttcacagactggaagaatctttccttgtggaagaagataagaaacacgagaggcaccctatttttggcaatatcgtggatgaggtggcttaccacgaaaaataccctacaatataccacctcaggaaaaaattggttgatagtacagacaaggccgacctcaggctcatctatttggccctggcccatatgattaaattcagggggcactttctcatcgagggagatttgaaccccgacaacagtgatgttgataagctctttattcagctcgtgcagacttacaatcagttgtttgaggaaaaccccattaatgcttccggggtggacgccaaggcaatcctttctgcaagactctcaaagtcaaggagactcgaaaatctgatagcacagcttccaggagagaagaagaacgggctctttggaaacctgatcgctctgtcactcggactcacacccaatttcaaaagcaattttgatttggcagaggacgctaagctgcaactcagtaaggatacctacgacgatgacttggataatctgctcgcacaaattggggaccagtatgcagacctgtttctcgcagctaagaacttgagtgacgccatattgctcagtgacatcctcagggttaataccgagattacaaaagctccactctctgcaagcatgatcaagaggtatgacgagcaccatcaagacctgacactccttaaggcgttggttaggcagcaacttcctgaaaagtataaggaaatcttcttcgatcaaagcaaaaacggctacgccggctatatagacgggggagcatcccaagaagaattttataagttcataaaacctatattggagaagatggacgggacagaggaattgctcgtgaaactgaacagggaggatctcctcaggaagcaaaggaccttcgacaatggctccatcccacatcagattcacctcggcgaactgcacgcaatactgagaagacaagaggacttttatcctttcctgaaggacaacagggagaaaatcgagaaaatcttgacattcagaatcccatactacgttgggcctctggccagaggtaacagtaggttcgcctggatgactaggaaatcagaggagactattacaccctggaactttgaagaagttgttgataagggagcttcagcacaatcattcatcgaaagaatgacaaactttgacaaaaatctgcctaatgagaaagtgctcccaaaacattccctgctgtatgagtattttaccgtttataacgagcttaccaaggtgaaatacgttactgaaggtatgagaaagccagcttttctttcaggggagcaaaagaaggctatcgtggatcttctctttaagaccaacagaaaggttaccgtgaagcagcttaaggaagactactttaaaaagatcgagtgttttgactcagtggaaataagcggtgttgaagatagattcaacgcatccttgggaacttatcatgatcttcttaagataatcaaggataaagactttctcgacaacgaggaaaacgaagatatactggaggacatagttctgacacttactttgttcgaggatagggagatgatcgaggaaagactgaaaacatatgctcaccttttcgacgacaaagttatgaaacaactcaagagaaggagatatacagggtgggggagattgagcaggaaactgattaatggtatcagagacaaacagtcaggaaaaacaatactcgactttttgaaatcagacgggttcgcaaataggaatttcatgcagcttatacacgacgattcacttacttttaaagaggacattcaaaaggctcaagttagtggacaaggtgactccctccacgaacacatcgcaaatctcgctggcagccctgcaattaagaagggtatactccagacagttaaggttgttgacgagctggttaaagtgatgggaagacacaaacccgagaacatagtgatagagatggccagggaaaaccaaaccactcaaaaagggcagaaaaattccagagagaggatgaaaaggattgaagaaggtatcaaggagctgggtagccaaattctgaaagaacatcctgtggaaaacactcaactccagaatgagaaactctatctgtactatctgcaaaatgggagagatatgtatgtggaccaggaactggacataaacaggctctcagattacgatgtggatcatatcgtgccacagtcctttcttaaggatgatagcatcgacaataaggtgcttaccaggtccgacaagaacaggggaaagtcagataacgtgccttctgaagaagttgttaaaaagatgaagaactactggagacagctgcttaacgctaagctcataacacagaggaagtttgacaacttgaccaaggccgagagaggcggactctcagaattggataaggcagggttcataaaaaggcagctggtggaaacaaggcagataactaaacatgtggctcagatcctcgatagtaggatgaatacaaaatacgatgagaacgacaagctcataagggaggttaaagtgataactctgaaatccaaactggttagcgattttaggaaggatttccagttttacaaagttagggagatcaacaattatcatcacgcccacgatgcctacttgaacgcagttgtgggtactgcacttatcaaaaagtaccctaagctggaatccgagtttgtttatggagactataaggtgtacgacgttagaaaaatgattgcaaagtcagagcaggagatagggaaagccactgcaaaatatttcttttatagcaatatcatgaatttctttaagacagaaatcacactggccaatggggaaataaggaagaggcccctgatcgaaactaatggcgagacaggggagattgtgtgggataaaggtagggactttgcaacagtgaggaaagtgctgagcatgccccaagttaatatcgttaaaaagaccgaggttcaaacagggggctttagtaaggaaagcattttgcccaagaggaatagtgacaaattgattgctaggaaaaaagattgggaccccaaaaagtatggcggatttgatagccccactgttgcttactccgtgctcgtggttgcaaaggtggagaagggaaagagcaagaaactgaagtcagttaaggaactccttggtatcactatcatggaaagaagctcctttgagaagaaccctattgacttcctggaggctaaagggtacaaagaggttaagaaagaccttatcattaaattgcccaaatatagtcttttcgagcttgaaaacggaagaaagaggatgcttgcatccgctggcgaattgcaaaagggcaatgagcttgctctcccttccaagtatgtgaacttcctttatcttgcctcacactatgaaaaactcaaaggttcacccgaagacaacgaacaaaagcaactatttgtggaacaacacaagcactacctggacgaaatcattgagcaaatttctgagttttcaaaaagggtaatcttggctgacgcaaatctcgacaaagttttgtcagcttacaacaaacatagagataagccaattagagagcaagctgagaatatcatccatctgtttaccctgactaaccttggagcgcctgctgcttttaaatatttcgacaccacaatcgacaggaagaggtacactagcactaaggaagttctcgacgccaccctcatccaccagagtattacaggcctgtacgagacaagaattgatctttctcaacttggtggtgac

SEQ ID NO: 11 - DNA, 4101 bp, synthetic construct
  misc_feature 1..4101: note = Cas9 nucleic acid sequence
  source 1..4101: mol_type = other DNA
gataagaagtactcaatcggtctggcaatcggaaccaactctgtgggttgggcagtgattacagatgagtataaggtgccaagcaaaaaattcaaggtgctgggtaataccgacagacacagcattaagaagaatttgattggagcactcctctttgactcaggggaaacagcagaggcaacaaggctgaagaggacagcaaggcggaggtacacaaggcggaaaaacaggatatgctacctccaggaaatctttagcaacgagatggctaaagtggatgatagctttttccatagactcgaagaatcctttcttgttgaagaggacaaaaagcatgaaaggcatcccatcttcggcaatatagttgatgaggttgcataccatgagaagtaccccacaatctaccacctcagaaagaaacttgtggactccacagataaagcagacctgaggctcatatacctcgcactcgcacacatgatcaagttcagagggcactttctcatcgaaggtgacctgaatccagataattcagatgtggataaactgtttatacagctggtgcaaacatacaaccaacttttcgaggaaaacccaatcaatgcctccggtgttgatgcaaaggccatcctgtcagcaagactcagcaaaagcaggcggctcgaaaacctcatcgcccagcttcccggtgaaaagaagaacgggctctttggtaatctcatcgcattgagccttggtcttactccaaacttcaagagcaattttgatctggcagaggatgctaaactgcaactctcaaaggacacatatgacgatgaccttgacaatctgttggcccagatcggggaccaatatgcagacctcttcctggccgcaaagaatctgtcagatgcaatcctcttgtccgacatactgagagttaacactgagatcacaaaggcacctctgtccgcctccatgattaagagatacgatgagcatcaccaggatctgactttgctcaaagccctcgttagacagcagttgccagaaaagtacaaagaaatattctttgatcaatcaaaaaacggatatgcagggtacatcgacggtggggcaagccaggaagagttctacaaattcatcaaacctatcctggaaaagatggatgggacagaagagctgctggttaagctgaatagggaagacctcctcagaaagcagaggacatttgataacgggagcatccctcatcaaatccacctcggtgaactccatgctatcctgagaaggcaggaagacttttatccatttttgaaggacaatagggagaaaatcgaaaaaatcctgacattcagaatcccatactacgttggtcctctggcaagaggtaacagtaggttcgcatggatgacaaggaaaagcgaggagacaatcacaccctggaattttgaggaagttgttgacaagggtgccagcgcacaatcctttatcgaaagaatgacaaatttcgacaagaatctgcctaacgaaaaggttctcccaaagcattcactcctgtacgaatattttacagtttataacgaactgactaaagttaaatacgttaccgagggtatgaggaagccagcattcctttccggggaacagaagaaagctattgtggacctcctgttcaagacaaatagaaaagtgacagttaagcaactcaaagaggattacttcaaaaagatcgaatgttttgactctgtggagatcagcggggtggaggatagattcaacgccagcctgggtacatatcatgatctcctgaaaatcattaaagacaaggacttccttgacaacgaggagaacgaggacattctggaagacattgttctgaccctcacactctttgaggatagggagatgattgaggaaagactgaagacctacgcccacctctttgacgataaagtgatgaaacagctcaagagaagaaggtatacaggttgggggagactgagcaggaagttgatcaatgggattagggacaaacagtccgggaaaacaatcctcgattttctgaagtcagacggtttcgcaaacagaaattttatgcagctcattcacgatgacagcttgacattcaaggaagacatccaaaaggctcaagtgagcggccaaggggatagcctccacgagcatattgcaaatctggcaggttcaccagccatcaaaaagggcatacttcagacagttaaggttgtggacgaattggttaaagttatgggcaggcataagccagagaatatcgttatcgaaatggcaagggagaaccaaacaactcaaaaagggcagaaaaatagcagagagaggatgaaaagaatcgaggaagggatcaaggaacttgggtcccaaatcctcaaggagcacccagttgaaaatactcaactgcaaaacgagaagctctatctctactatctccaaaacgggagggatatgtatgttgaccaggagctggatattaacagactgtcagattatgatgttgatcatatcgtgccccagtcattcctgaaggacgattccatcgacaacaaagttctcacaaggtccgataaaaacaggggcaagtccgataacgttccaagcgaagaagtggtgaaaaagatgaaaaactattggagacaacttctgaatgcaaagttgattactcagagaaagtttgacaacctcacaaaagcagaaagaggcgggcttagcgaactcgataaggcagggtttatcaaaagacagctggttgagacaaggcagatcacaaaacatgtggcacagatccttgactcaaggatgaataccaagtatgatgagaatgataagttgatcagggaggttaaagttatcacactcaaatccaaactggtgtcagacttcaggaaagactttcaattttataaggtgagggagatcaataactaccaccatgcacatgacgcctacctgaacgcagtggtgggtacagcattgattaaaaaataccctaagctggagtctgagtttgtgtacggggactacaaggtgtacgacgtgaggaaaatgatagccaagtccgagcaggagatcgggaaagcaacagctaagtatttcttttacagtaatatcatgaatttctttaaaactgagattactctggcaaacggggagatcaggaaaagacccctcatcgagactaatggtgaaacaggtgagatcgtttgggacaaggggagggattttgctactgttagaaaagttctgagtatgccacaagtgaatattgtgaaaaagacagaagttcagacaggtgggttctccaaagaatccatcctgcccaagagaaattcagacaagctcatcgcaagaaagaaggactgggaccctaagaagtacggaggatttgacagccccaccgtggcctattccgtgcttgttgtggcaaaggtggagaaagggaagagcaaaaaactgaaatccgtgaaagaactgctgggaattaccatcatggaaagaagctcctttgagaagaacccaatcgacttcctggaagcaaaaggatataaggaagtgaaaaaggacctcattatcaagctcccaaaatactcacttttcgagttggagaacggtagaaagaggatgctggcaagcgcaggggaacttcagaaaggcaatgagctggcattgccatcaaagtatgtgaacttcctctacttggccagccattacgagaaacttaaaggtagcccagaagataacgagcaaaaacagctctttgtggaacagcataagcattatctggatgagatcatagaacaaatctcagagttttccaagagagttatcctcgcagatgcaaacctggataaggttctctcagcctataataagcatagagacaagccaattagagagcaagcagagaacattatccacttgttcactcttacaaacctgggggcaccagccgccttcaaatatttcgatacaacaatagacagaaagaggtataccagcaccaaagaagttctcgacgccacactgatccatcaatcaatcacaggcctttacgaaactaggatcgacttgtcacaactgggtggggat

SEQ ID NO: 12 - DNA, 3307 bp, synthetic construct
  misc_feature 1..3307: note = Cas9 nucleic acid sequence
  source 1..3307: mol_type = other DNA
gagcaaggacacctacgacgacgacttggacaacctattggcccagataggtgaccagtatgcagacctcttccttgcggccaagaacttgagtgacgctatactgctcagtgacatcctgagggtgaacactgagatcactaaggcccctctctctgcctcaatgattaagcgttacgacgagcatcaccaggatctcaccctgcttaaggcccttgttcggcagcagctccctgagaagtacaaggagatattttttgaccagtctaagaacggctacgccggttacattgacggtggggcaagccaggaggagttctacaagttcatcaagccgatccttgagaagatggacggcaccgaggagctacttgtcaagttgaaccgggaagacctgctccggaaacagcgtacattcgacaacggcagcatccctcaccagatccacctgggcgaactacacgccatcctccgacgtcaggaggacttctatccattcttgaaagataacagggaaaaaatcgaaaaaatacttacgtttcgaataccttactacgtggggccccttgctcggggaaactccagattcgcatggatgaccaggaagtcagaggagaccatcacaccctggaactttgaggaggtggttgacaaaggtgcttctgcccagtccttcattgagcggatgactaacttcgacaagaacctgcccaacgagaaggtgctgccaaagcacagcctgctctacgaatactttactgtgtacaatgagctgacgaaggtgaagtacgtgacagaggggatgcggaagcccgctttcctgagcggcgagcaaaaaaaagcaatcgtggacctactgttcaagaccaaccgaaaggtgacagtgaagcagctcaaggaggactacttcaaaaaaatcgagtgcttcgactctgttgagataagcggcgtggaggaccgattcaacgcctcattgggaacctatcacgacctgctcaagatcattaaggacaaggacttcctggataatgaggagaatgaggacatcctggaggatattgtgctgacccttactctattcgaggacagggagatgatcgaggagcgactcaagacctacgctcacctgttcgacgacaaggttatgaagcaattgaagcgtaggcgatacacggggtggggaagactctcccgaaaactgataaacggcatcagggacaagcagtcagggaagacgatcttggacttcctgaaatccgacgggttcgccaaccgcaacttcatgcagctcattcacgacgactcactaacgttcaaagaggacattcagaaggctcaagtcagtggacaaggcgactccctgcacgagcacattgcaaaccttgcgggctccccggcgattaaaaagggcattctccaaacggttaaggtggtggacgagctggtgaaggtgatgggccgacacaagcctgagaacatcgtgatcgagatggccagggagaaccagactacccagaagggtcagaagaactctcgggaacgtatgaagcgtattgaggaggggattaaggagttgggctctcaaatcctcaaggagcaccctgtggagaacactcagctccaaaacgagaagctgtacctgtactacctgcaaaacgggcgcgatatgtacgtggatcaggagttggacatcaacaggcttagcgattacgacgtggaccacatcgtgccacagtcattcttaaaggacgacagcatcgacaacaaggttctgacgaggagcgacaagaatcgagggaaaagtgacaatgttccatccgaggaggtggtcaagaaaatgaagaactattggcgtcagcttctgaacgccaagctcatcacccagcggaaattcgacaacctgactaaggctgagcgaggcggactctccgagcttgacaaggctggcttcatcaagcggcagttggtcgaaacccgacagataacgaagcacgttgcccagatacttgactcccgtatgaacaccaagtacgacgagaacgacaagctcatcagggaggtgaaggtcattacccttaagtccaaactcgtcagcgactttcgtaaggacttccagttctacaaggtgcgcgagatcaataactaccaccacgcacacgacgcctacctgaacgcagtggttggaaccgcgttgattaaaaagtaccccaagttggagtcggagttcgtttacggggactacaaggtgtacgacgttcggaagatgatcgccaagtctgaacaggagatcgggaaagcaaccgccaagtatttcttctatagcaacatcatgaacttctttaaaaccgagatcacacttgccaatggcgagatccgtaagaggccgctgatcgagacaaatggggagactggcgagatcgtgtgggacaagggccgcgacttcgcaaccgttcggaaagtcttgtccatgcctcaagtcaacatcgtcaagaagactgaggtgcaaacaggcgggttctcgaaggagtccatactgcccaagaggaactcagacaagctcatagcacgcaaaaaagactgggatccaaagaaatacggcgggttcgactcgccgacagtcgcatactccgtgttagtggtggctaaagtggaaaaggggaagtccaagaagctcaagtccgtcaaggagttgctcgggatcaccattatggaacggtcctcattcgagaagaatcccattgacttcctagaggcgaagggctacaaagaggtcaaaaaggacctaattattaagctccccaagtattcactcttcgaacttgaaaatggtcgtaagcggatgttggcaagcgctggagagcttcagaaggggaacgagcttgcactgccttccaagtacgtgaacttcctgtacctcgcctctcattacgagaagttgaagggctcaccggaggacaacgagcagaagcagttgttcgtggagcagcacaagcactacctcgacgagatcattgagcagataagtgagttcagcaaacgggtgatccttgccgacgctaacctggacaaggtgctgagcgcctacaacaagcacagagacaagccgatccgagagcaagcggagaacatcatacacctgttcaccctcacgaacctcggggctcccgcagccttcaaatattttgacacgaccatcgaccgtaaacgctacactagcacgaaggaggtgctggacgctacccttatccaccagtccatcaccggcctgtacgagacgagaatcgacttgtcgcagctcggtggtgac

SEQ ID NO: 13 - DNA, 4101 bp, synthetic construct
  misc_feature 1..4101: note = Cas9 nucleic acid sequence
  source 1..4101: mol_type = other DNA
gacaaaaaatactcaattggtctggcaattgggaccaacagtgtcggatgggccgtgattaccgacgagtacaaggtgccgtccaaaaaattcaaggtgcttgggaacaccgaccgccactcgatcaagaaaaacctaatcggtgcgttgcttttcgacagtggggagaccgccgaggcaacacgcttaaaacgcacagctaggaggagatatacacggcgcaagaaccgaatatgctacttacaggagatattctccaatgagatggcgaaggtggacgactctttcttccatcggcttgaggaatccttcctggtcgaggaggacaagaagcacgagcgacacccgatattcgggaacatcgttgatgaggtggcgtaccacgagaagtacccaacgatataccacttacgcaagaagctcgtggactctacggacaaggccgacttgcgccttatctacttggcactggcccacatgattaagttccgaggccacttccttatcgagggtgacctgaaccccgataactccgacgtggacaagctcttcatccaactcgtccagacatacaaccagctattcgaggagaatcctatcaacgcctctggggtggacgctaaagctatcctctcagcccgcctgtcaaagtcgaggaggttggagaacctaatcgcccagcttccaggcgagaagaaaaatgggctgttcggaaaccttatcgcactctcactgggcctaaccccgaacttcaagtccaacttcgacctggcagaggacgcgaaattgcagttgtcgaaagacacctatgacgatgacctggacaacctgttggcccagataggggaccagtacgccgacctgttcctagcggccaagaacctgtccgacgccatcttgctgtcggatatactgcgggtgaacaccgagatcactaaagcacctctctccgccagcatgattaagcgttacgacgagcaccaccaagatttgaccctgctaaaggcacttgtacggcagcagcttcccgagaagtacaaggagatctttttcgaccaaagcaagaacggctacgccgggtacatcgacggaggtgccagccaggaggagttctacaagttcattaagcccatcctggagaagatggacgggactgaggaactacttgtgaagctgaaccgggaagacttactacggaagcagcgtaccttcgacaacggttctatcccacatcagatccatcttggggagttgcacgcgatcctgcgacgccaggaggacttttaccccttcctgaaagacaaccgcgagaaaatcgagaagatactgaccttcagaataccttactacgtcggaccccttgcgcgaggcaactcaagattcgcgtggatgaccaggaaatcagaggagaccatcacaccctggaatttcgaggaggtggttgacaagggtgcctccgcccagtcctttatcgaacgaatgaccaacttcgacaagaacttgcccaacgagaaggtgctccccaaacacagcctcctctacgaatatttcacagtgtacaacgagcttactaaagttaagtatgttactgagggcatgaggaaacccgccttcctgtcaggcgagcagaagaaagctattgtggacctccttttcaagaccaaccggaaggtgacagtgaagcagctcaaggaggactacttcaagaagatagagtgcttcgacagcgtggagatcagcggggtggaggacagattcaatgcctctctcggaacataccacgacttgcttaagatcatcaaggacaaggacttcctcgacaacgaggaaaacgaggatattctggaggatattgttctgactcttaccctgttcgaggaccgggagatgatcgaggagcgtctcaagacctacgcccacctgttcgacgacaaagttatgaagcagctcaagcgtcggagatataccggatggggccgtctgtctcggaagctcatcaacgggatcagggacaagcagtcagggaagacgatcttagacttccttaagtctgacggcttcgccaacaggaacttcatgcagttgatccacgacgacagccttaccttcaaggaggacatccagaaggcccaagtgagtggccagggtgacagcctccacgagcatattgctaatcttgcgggttccccagcgattaaaaagggcatacttcaaaccgttaaggtggtggacgagcttgtcaaggtgatggggcgacacaagcccgagaacatcgtgatcgagatggccagggagaaccagaccacccagaaggggcagaagaatagccgagaacgcatgaagcgcatcgaggaggggattaaggagctagggagccagatcctcaaggaacatcccgtcgagaacacccagctccagaacgagaagctatacctctactacttgcaaaacgggagggatatgtacgtggatcaggagttggacattaaccgcctaagcgactacgacgtagatcacatcgtgcctcagtcattcctcaaagacgacagcattgacaacaaagtcttgacccgatccgacaagaaccgaggaaaatccgacaatgtgccctcagaggaggtcgtcaagaaaatgaagaactattggaggcagctacttaacgccaaactcataacccagcggaagttcgacaacctgacaaaggctgagcggggtgggctcagcgagcttgacaaggctggcttcatcaagcggcagttggtggagacaagacagataacgaagcacgtggctcagatcctggactctcgcatgaacacgaagtacgacgagaacgacaaattgatccgcgaggtcaaggttattacgctcaagagcaaacttgtcagcgatttccgcaaggacttccagttctacaaggtgagggagattaacaactaccaccatgcacatgatgcctacttgaacgcagtggtggggaccgcgcttattaaaaagtaccctaagttggagtcagagttcgtttatggggactacaaggtgtacgacgtccggaagatgattgcaaagtctgaacaggaaatcgggaaggccaccgccaaatatttcttctacagtaacattatgaatttttttaagactgaaattactctcgcaaacggcgagatcaggaagcgtcccctcatcgagacaaacggggagaccggggagatagtctgggacaaggggcgggacttcgctacggtgaggaaggtgctctcgatgccacaagtgaacatcgtcaaaaagacagaggtgcagaccggtggcttctcaaaggagtcaatcctgccaaaacgtaacagcgacaagctcatcgcccgcaagaaagactgggaccctaagaagtatggtgggttcgactcaccgacggtcgcatactccgttctggtcgtggcaaaggtggaaaagggcaagtccaaaaaactgaaatccgtgaaggagttgcttggcattaccatcatggaacgcagcagcttcgagaagaaccccattgacttcctggaggctaaagggtacaaggaggtcaagaaagatttaattattaagctacctaagtacagcttgttcgagctggagaacggccgaaaacgaatgctcgcatccgccggggaacttcaaaagggcaacgagcttgcgctgccctccaagtacgtgaacttcctgtacttggcatcccactacgagaaactcaagggtagcccagaggacaacgagcagaagcagctattcgtggagcagcacaagcactacctcgacgagataatcgagcagatcagtgagttcagtaagcgggtgatactcgcggacgccaacttggacaaggtgcttagtgcctacaacaagcaccgtgacaagcccatccgagaacaggctgagaacatcatccaccttttcactctgacaaacctcggtgctcccgccgccttcaaatacttcgacactaccatcgacaggaagcgctacacatctacgaaggaagttcttgacgctacgcttattcatcagtctatcacagggctgtacgagacaaggatcgaccttagccaactcggcggggat

SEQ ID NO: 14 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 15 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 16 - protein, 1228 aa, unidentified
  source 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 17 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 18 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 19 - protein, 1238 aa, Candidatus Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 20 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 21 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 22 - protein, 1206 aa, unidentified
  source 1..1206: note = Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 23 - protein, 1233 aa, unidentified
  source 1..1233: note = Lachnospiraceae bacterium
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 24 - protein, 1227 aa, unidentified
  source 1..1227: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 25 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 26 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 27 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 28 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 29 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 30 - protein, 1484 aa, unidentified
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 31 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 32 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 33 - DNA, 3987 bp, synthetic construct
  misc_feature 1..3987: note = Cas12a polynucleotide
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 34 - DNA, 3987 bp, synthetic construct
  misc_feature 1..3987: note = Cas12a polynucleotide
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 35 - DNA, 3987 bp, synthetic construct
  misc_feature 1..3987: note = Cas12a polynucleotide
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 36-38 - skipped (3 entries)

SEQ ID NO: 39 - protein, 24 aa, synthetic construct
  REGION 1..24: note = GCN4 sequence
EELLSKNYHLENEVARLKKGSGSG

SEQ ID NO: 40 - protein, 241 aa, synthetic construct
  REGION 1..241: note = GCN4 sequence
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 41 - protein, 277 aa, synthetic construct
  REGION 1..277: note = ScFv antibody
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 42 - DNA, 66 bp, Saccharomyces bayanus
  source 1..66: mol_type = genomic DNA
ttcttgtcgtacttatagatcgctacgttatttcaattttgaaaatctgagtcctgggagtgcgga

SEQ ID NO: 43 - protein, 609 aa, Homo sapiens [sequence omitted]

SEQ ID NO: 44 - protein, 482 aa, synthetic construct
  REGION 1..482: note = polypeptide
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 45 - DNA, 10 bp, Methanobacterium thermoautotrophicum
  source 1..10: mol_type = genomic DNA
aatttttgga

SEQ ID NO: 46 - protein, 83 aa, Methanobacterium thermoautotrophicum
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 47 - DNA, 25 bp, Bacteriophage MS2
  source 1..25: mol_type = genomic DNA
gcgcacatgaggatcacccatgtgc

SEQ ID NO: 48 - protein, 116 aa, Bacteriophage MS2
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 49 - DNA, 26 bp, Bacteriophage PP7
  source 1..26: mol_type = genomic DNA
ataaggagtttatatggaaaccctta

SEQ ID NO: 50 - protein, 127 aa, Bacteriophage PP7
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 51 - DNA, 19 bp, Shigella flexneri
  source 1..19: mol_type = genomic DNA
ctgaatgcctgcgagcatc

SEQ ID NO: 52 - protein, 62 aa, unidentified
  source 1..62: note = Shigella phage
MKSIRCKNCNKLLFKADSFDHIEIRCPRCKRHIIMLNACEHPTEKHCGKREKITHSDETVRY

SEQ ID NO: 53 - DNA, 6147 bp, synthetic construct
  misc_feature 1..6147: note = nCas9::GCN4::P2A::EGFP
  source 1..6147: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacggaggcggaggatctggaggcggtgggagcggaggcggtgggtctggacctaagaagaagagaaaggtggccgcagctggctccgaggaactgctgtccaagaactatcacctggagaacgaggttgcaagactgaagaagggatctggctcaggaggctctggcagcggtgggtccggttcagggtctggaggcagcggttccggtgggtcaggatctggtgaggaactgctgtctaagaactaccatctggagaacgaggttgctaggctgaagaagggtagcgggtccggaggctcaggatctggtgggagcggatctggctcaggaggctctggcagcggtgggtccggttcaggtgaagagttgctgagcaagaactaccacctggagaacgaggtagccagactgaagaagggatctggcagcggaggctccggatcaggtgggtctggtagcgggtccggaggctcaggctctggtgggagcggatctggtgaagagttgttgtccaagaactaccatctggagaacgaggtagcaaggctgaagaagggttcagggtctggaggcagcggatctggaggatctggatctggcagcggaggctccggctcaggtgggtctggtagcggcgaagagttgttgtctaagaactaccacctggagaacgaggtcgcgagactgaagaagggatctggctcaggaggctctggaagcggtgggtccggttcagggtctggaggcagcggctccggtgggtcaggatctggagaagagcttttgagcaagaactaccatctggagaacgaggtcgccaggctgaagaagggtagcgggtccggaggctcaggatctggtgggagcggatctggctcaggaggctctggcagcggtgggtccggttcaggcgaagagttgctttctaagaactaccacctggagaacgaggtagcgagactgaagaagggatctggcagcggaggctccggatcaggtgggtctggtagcgggtccggaggctcaggctctggtgggagcggatctggagaagagctgctgtctaagaactatcatctggagaatgaggttgcaaggctgaagaagtctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctggtggttctcccaagaagaagaggaaagtctaa

SEQ ID NO: 54 - DNA, 3996 bp, synthetic construct
  misc_feature 1..3996: note = scFV::MuLV(5M)::GB1::P2A::EGFP
  source 1..3996: mol_type = other DNA
atgaaacggacagccgacggaagcgagttcgagtcaccaaagaagaagcggaaagtcggtccagacatcgttatgactcaatccccctcaagcctttcagcctccgtgggcgacagagttaccattacctgccgctcaagcactggagcagtgaccacttctaactacgctagctgggtgcaggagaagccaggaaagctgttcaaggggctgattggaggcacaaacaatagagcccctggcgtgccatccaggttttctggaagcctgattggcgataaggcaacactgaccatctctagcctgcagcccgaggacttcgctacctacttttgcgccctgtggtatagcaatcactgggtgttcggtcaggggactaaggtggaactgaagagaggtgggggaggctccggtgggggaggctcaggtgggggaggctcctcaggtgggggatctgaggtgaagctgctggaaagcggcggtgggctggtgcagcccggaggctccctgaagctgtcatgtgccgtgtctggattttcactgaccgattacggcgtgaactgggtgagacaggcccctggaaggggactggagtggattggagtgatttggggagatgggatcacagactacaattccgcactgaaggatagattcattatctcaaaggacaacggaaagaacaccgtgtatctgcagatgtctaaggtgaggagcgatgacactgccctgtactattgcgtgacagggctgtttgactattgggggcaaggcactctggttaccgtgagcagctatccatacgatgttcccgactacgctgggtctagcgagacacctggaacctctgaaagcgccaccccagagggaggctccggtgggtccggatcaacacttaatattgaggatgaacatagattgcacgagacctctaaggaacctgatgtttctcttggatcaacttggttgtcagatttcccacaagcatgggcagagaccggaggtatgggtcttgctgttaggcaggcaccacttattattcctttgaaggcaacctctactcctgtgtcaattaagcaatatccaatgtctcaggaagctaggcttggaattaagcctcacattcaaagacttttggatcagggtattttggtgccatgtcaatcaccttggaacacaccacttttgcctgttaagaagcctggaactaatgattacagaccagtgcaagatttgagggaggttaacaagagagtggaagatattcacccaactgttccaaacccttataatcttttgtctggattgccaccttcacatcaatggtacactgtgcttgatttgaaggatgcatttttctgccttaggttgcatccaacatctcagcctctttttgctttcgagtggagagatcctgaaatgggaatttctggtcaacttacatggaccaggttgcctcagggtttcaagaactcaccaaccttgtttaatgaggcacttcacagggatttggctgattttaggattcaacatcctgatcttatccttttgcagtatgttgatgatcttttgcttgctgcaacttctgaattggattgtcaacagggaactagggcattgcttcaaacacttggaaatttgggttacagagcttcagcaaagaaggctcagatttgccaaaagcaggttaagtatcttggatacttgcttaaggaaggacaaaggtggttgaccgaggctagaaaggaaactgtgatgggtcaaccaacacctaagacccctaggcagcttagagagttcttgggaaaggcaggtttttgtaggcttttcattccaggatttgctgaaatggctgcaccactttatcctttgaccaagcctggaactttgtttaactggggtccagatcaacagaaggcataccaagaaattaagcaggctttgcttactgctccagcacttggtttgcctgatcttacaaagccatttgagttgttcgttgatgaaaagcaaggatatgcaaagggtgtgcttacccagaagttgggaccttggagaaggcctgttgcttacctttctaagaaacttgatccagtggctgcaggttggccaccttgtcttagaatggttgctgcaattgcagtgcttacaaaggatgctggaaagttgactatgggacaacctcttgttattttggcaccacacgctgttgaggcacttgtgaagcagccacctgataggtggttgtcaaacgcaagaatgacccattatcaagctcttcttttggatactgatagggtgcagttcggtcctgttgtggctttgaatccagcaacacttttgccacttcctgaggaaggattgcaacacaactgccttgatattttggctgaggcacatggtacaagacctgatcttaccgatcagccattgcctgatgctgatcacacttggtacacagatggatcttcacttttgcaagaaggacagaggaaggctggtgctgcagttactacagagactgaagtgatttgggctaaggcacttccagctggaacatctgctcaaagagcagagcttattgctttgacccaggcacttaagatggctgaaggaaagaagttgaacgtttacactgattctaggtatgctttcgcaacagctcatattcacggagaaatctatagaaggagaggatggttgacatcagagggaaaggaaattaagaacaaggatgaaattcttgcacttttgaaggctctttttcttcctaagagattgtctattattcattgcccaggacaccaaaagggtcattcagcagaagctaggggaaatagaatggctgatcaggctgcaagaaaggctgcaattactgagacacctgatacctctactcttttgatcgaaaactcttcaccaagcggaggatccggaggatctggaggcagctacaagctgattctgaacggaaagaccctgaagggagagaccactacagaagcagtggatgctgccacagctgagaaggtgttcaagcagtacgccaacgataacggagtggacggcgagtggacctatgatgacgcaaccaagacttttacagtgaccgaatctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctggtggttctcccaagaagaagaggaaagtctaa

SEQ ID NO: 55 - DNA, 126 bp, synthetic construct
  misc_feature 1..126: note = pegRNA
  source 1..126: mol_type = other DNA
ggaatcccttctgcagcaccgttttagagctagaaatagcaagttaaaataaggctagtccgttatcaacttgaaaaagtggcaccgagtcggtgcggaaaagcgatcaaggtgctgcagaaggga

SEQ ID NO: 56 - DNA, 127 bp, synthetic construct
  misc_feature 1..127: note = pegRNA
  source 1..127: mol_type = other DNA
ggaatcccttctgcagcaccgttttagagctagaaatagcaagttaaaataaggctagtccgttatcaacttgaaaaagtggcaccgagtcggtgcggaaaagcgagccaggtgctgcagaagggat

SEQ ID NO: 57 - DNA, 130 bp, synthetic construct
  misc_feature 1..130: note = pegRNA
  source 1..130: mol_type = other DNA
ggaatcccttctgcagcaccgttttagagctagaaatagcaagttaaaataaggctagtccgttatcaacttgaaaaagtggcaccgagtcggtgcggaaaagcgatccaatcggtgctgcagaagggat

SEQ ID NO: 58 - DNA, 126 bp, synthetic construct
  misc_feature 1..126: note = pegRNA
  source 1..126: mol_type = other DNA
ggaatcccttctgcagcaccgttttagagctagaaatagcaagttaaaataaggctagtccgttatcaacttgaaaaagtggcaccgagtcggtgcggaaaagcgatcaggtgctgcagaagggat

SEQ ID NO: 59 - DNA, 125 bp, synthetic construct
  misc_feature 1..125: note = pegRNA
  source 1..125: mol_type = other DNA
gtcatcttagtcattacctggttttagagctagaaatagcaagttaaaataaggctagtccgttatcaacttgaaaaagtggcaccgagtcggtgcaacgaacaccgcaggtaatgactaagatg

SEQ ID NO: 60 - DNA, 125 bp, synthetic construct
  misc_feature 1..125: note = pegRNA
  source 1..125: mol_type = other DNA
gtcatcttagtcattacctggttttagagctagaaatagcaagttaaaataaggctagtccgttatcaacttgaaaaagtggcaccgagtcggtgcaacgaacaccccaggtaatgactaagatg

SEQ ID NO: 61 - DNA, 125 bp, synthetic construct
  misc_feature 1..125: note = pegRNA
  source 1..125: mol_type = other DNA
gtcatcttagtcattacctggttttagagctagaaatagcaagttaaaataaggctagtccgttatcaacttgaaaaagtggcaccgagtcggtgcaacgaacacatcaggtaatgactaagatg

SEQ ID NO: 62 - DNA, 125 bp, synthetic construct
  misc_feature 1..125: note = pegRNA
  source 1..125: mol_type = other DNA
gtcatcttagtcattacctggttttagagctagaaatagcaagttaaaataaggctagtccgttatcaacttgaaaaagtggcaccgagtcggtgcaacgaacatctcaggtaatgactaagatg

SEQ ID NO: 63 - DNA, 126 bp, synthetic construct
  misc_feature 1..126: note = pegRNA
  source 1..126: mol_type = other DNA
gcattttcaggaggaagcgagttttagagctagaaatagcaagttaaaataaggctagtccgttatcaacttgaaaaagtggcaccgagtcggtgctgtctgaagcaatcgcttcctcctgaaaat

SEQ ID NO: 64 - DNA, 126 bp, synthetic construct
  misc_feature 1..126: note = pegRNA
  source 1..126: mol_type = other DNA
gcattttcaggaggaagcgagttttagagctagaaatagcaagttaaaataaggctagtccgttatcaacttgaaaaagtggcaccgagtcggtgctgtctgaaggcatcgcttcctcctgaaaat

SEQ ID NO: 65 - DNA, 129 bp, synthetic construct
  misc_feature 1..129: note = pegRNA
  source 1..129: mol_type = other DNA
gcattttcaggaggaagcgagttttagagctagaaatagcaagttaaaataaggctagtccgttatcaacttgaaaaagtggcaccgagtcggtgctgtctgaagccatccatgcttcctcctgaaaat

SEQ ID NO: 66 - DNA, 125 bp, synthetic construct
  misc_feature 1..125: note = pegRNA
  source 1..125: mol_type = other DNA
gcattttcaggaggaagcgagttttagagctagaaatagcaagttaaaataaggctagtccgttatcaacttgaaaaagtggcaccgagtcggtgctgtctgaagccatgcttcctcctgaaaat

SEQ ID NO: 67 - DNA, 120 bp, synthetic construct
  misc_feature 1..120: note = pegRNA
  source 1..120: mol_type = other DNA
gattcctggtgccagaaacagttttagagctagaaatagcaagttaaaataaggctagtccgttatcaacttgaaaaagtggcaccgagtcggtgcgtcaccactgtttctggcaccagg

SEQ ID NO: 68 - DNA, 120 bp, synthetic construct
  misc_feature 1..120: note = pegRNA
  source 1..120: mol_type = other DNA
gattcctggtgccagaaacagttttagagctagaaatagcaagttaaaataaggctagtccgttatcaacttgaaaaagtggcaccgagtcggtgcgtcacgcctgtttctggcaccagg

SEQ ID NO: 69 - DNA, 127 bp, synthetic construct
  misc_feature 1..127: note = pegRNA
  source 1..127: mol_type = other DNA
gattcctggtgccagaaacagttttagagctagaaatagcaagttaaaataaggctagtccgttatcaacttgaaaaagtggcaccgagtcggtgctcccgtcacccctgtgatttctggcaccagg

SEQ ID NO: 70 - DNA, 121 bp, synthetic construct
  misc_feature 1..121: note = pegRNA
  source 1..121: mol_type = other DNA
gattcctggtgccagaaacagttttagagctagaaatagcaagttaaaataaggctagtccgttatcaacttgaaaaagtggcaccgagtcggtgctcccgtcaccgtttctggcaccagg

SEQ ID NO: 71 - DNA, 2631 bp, synthetic construct
  misc_feature 1..2631: note = MCP::MuLV(5M)
  source 1..2631: mol_type = other DNA
atgaaacggacagccgacggaagcgagttcgagtcaccaaagaagaagcggaaagtcgcaagcaacttcactcagtttgtgctggtggacaacggaggaactggagatgtgacagtggctccatccaacttcgccaatggggtggcagagtggatttctagcaactctagaagccaggcctataaggtgacctgcagcgtgagacagtcctcagcacagaagaggaagtacactatcaaggtggaggtgcccaaggtggccacccagactgtgggtggggtggaactgcctgtggctgcctggaggtcatatctgaacatggagctgaccattcccatctttgcaactaattctgactgtgaactgattgtgaaggctatgcagggactgctgaaggatggcaaccctattccatccgccatcgcagctaattctggaatctactctgctggaggcggaggatctggaggcggtgggagcggaggcggtgggtctggacctaagaagaagagaaaggtggccgcagctggctccacacttaatattgaggatgaacatagattgcacgagacctctaaggaacctgatgtttctcttggatcaacttggttgtcagatttcccacaagcatgggcagagaccggaggtatgggtcttgctgttaggcaggcaccacttattattcctttgaaggcaacctctactcctgtgtcaattaagcaatatccaatgtctcaggaagctaggcttggaattaagcctcacattcaaagacttttggatcagggtattttggtgccatgtcaatcaccttggaacacaccacttttgcctgttaagaagcctggaactaatgattacagaccagtgcaagatttgagggaggttaacaagagagtggaagatattcacccaactgttccaaacccttataatcttttgtctggattgccaccttcacatcaatggtacactgtgcttgatttgaaggatgcatttttctgccttaggttgcatccaacatctcagcctctttttgctttcgagtggagagatcctgaaatgggaatttctggtcaacttacatggaccaggttgcctcagggtttcaagaactcaccaaccttgtttaatgaggcacttcacagggatttggctgattttaggattcaacatcctgatcttatccttttgcagtatgttgatgatcttttgcttgctgcaacttctgaattggattgtcaacagggaactagggcattgcttcaaacacttggaaatttgggttacagagcttcagcaaagaaggctcagatttgccaaaagcaggttaagtatcttggatacttgcttaaggaaggacaaaggtggttgaccgaggctagaaaggaaactgtgatgggtcaaccaacacctaagacccctaggcagcttagagagttcttgggaaaggcaggtttttgtaggcttttcattccaggatttgctgaaatggctgcaccactttatcctttgaccaagcctggaactttgtttaactggggtccagatcaacagaaggcataccaagaaattaagcaggctttgcttactgctccagcacttggtttgcctgatcttacaaagccatttgagttgttcgttgatgaaaagcaaggatatgcaaagggtgtgcttacccagaagttgggaccttggagaaggcctgttgcttacctttctaagaaacttgatccagtggctgcaggttggccaccttgtcttagaatggttgctgcaattgcagtgcttacaaaggatgctggaaagttgactatgggacaacctcttgttattttggcaccacacgctgttgaggcacttgtgaagcagccacctgataggtggttgtcaaacgcaagaatgacccattatcaagctcttcttttggatactgatagggtgcagttcggtcctgttgtggctttgaatccagcaacacttttgccacttcctgaggaaggattgcaacacaactgccttgatattttggctgaggcacatggtacaagacctgatcttaccgatcagccattgcctgatgctgatcacacttggtacacagatggatcttcacttttgcaagaaggacagaggaaggctggtgctgcagttactacagagactgaagtgatttgggctaaggcacttccagctggaacatctgctcaaagagcagagcttattgctttgacccaggcacttaagatggctgaaggaaagaagttgaacgtttacactgattctaggtatgctttcgcaacagctcatattcacggagaaatctatagaaggagaggatggttgacatcagagggaaaggaaattaagaacaaggatgaaattcttgcacttttgaaggctctttttcttcctaagagattgtctattattcattgcccaggacaccaaaagggtcattcagcagaagctaggggaaatagaatggctgatcaggctgcaagaaaggctgcaattactgagacacctgatacctctactcttttgatcgaaaactcttcaccatctggcggctcaaaaagaaccgccgacggcagcgaattcgagcccaagaagaagaggaaagtctaa

SEQ ID NO: 72 - DNA, 156 bp, synthetic construct
  misc_feature 1..156: note = FANCF gRNA with MS2 scaffold (O2 target)
  source 1..156: mol_type = other DNA
ggaatcccttctgcagcaccgttttagagctaggccaacatgaggatcacccatgtctgcagggcctagcaagttaaaataaggctagtccgttatcaacttggccaacatgaggatcacccatgtctgcagggccaagtggcaccgagtcggtgc

SEQ ID NO: 73 - DNA, 156 bp, synthetic construct
  misc_feature 1..156: note = FANCF gRNA with MS2 scaffold (O3 target)
  source 1..156: mol_type = other DNA
ccaaggtgaaagcggaagtagttttagagctaggccaacatgaggatcacccatgtctgcagggcctagcaagttaaaataaggctagtccgttatcaacttggccaacatgaggatcacccatgtctgcagggccaagtggcaccgagtcggtgc

SEQ ID NO: 74 - DNA, 5040 bp, synthetic construct
  misc_feature 1..5040: note = nCas9(H840A)::P2A::EGFP
  source 1..5040: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctggtggttctcccaagaagaagaggaaagtctaa

SEQ ID NO: 75 - DNA, 155 bp, synthetic construct
  misc_feature 1..155: note = FANCF pegRNA (O2 Target)
  source 1..155: mol_type = other DNA
ggggtcccaggtgctgacgtgttttagagctagaaatagcaagttaaaataaggctagtccgttatcaacttgaaaaagtggcaccgagtcggtgcaggtagtgtccgagaccgccgtgagctcggaaaagcgattgcggtgctgcagaagggat

SEQ ID NO: 76 - DNA, 193 bp, synthetic construct
  misc_feature 1..193: note = FANCF pegRNA (O3 Target)
  source 1..193: mol_type = other DNA
ggggtcccaggtgctgacgtgttttagagctagaaatagcaagttaaaataaggctagtccgttatcaacttgaaaaagtggcaccgagtcggtgcaggtagtgcttgagaccgccgtgagctcggaaaagcgattgcggtgctgcagaagggattccatgaggtgcgcgaaggaattacttccgctttcacc

SEQ ID NO: 77 - DNA, 82 bp, Homo sapiens
  source 1..82: mol_type = genomic DNA
ggaatcccttctgcagcacctggatcgcttttccgagcttctggcggtctcaagcactacctacgtcagcacctgggacccc

SEQ ID NO: 78 - DNA, 82 bp, synthetic construct
  misc_feature 1..82: note = Edit sequence for site O2
  source 1..82: mol_type = other DNA
ggaatcccttctgcagcaccgcaatcgcttttccgagctcacggcggtctcggacactacctacgtcagcacctgggacccc

SEQ ID NO: 79 - DNA, 120 bp, Homo sapiens
  source 1..120: mol_type = genomic DNA
ccaaggtgaaagcggaagtagggccttcgcgcacctcatggaatcccttctgcagcacctggatcgcttttccgagcttctggcggtctcaagcactacctacgtcagcacctgggaccc

SEQ ID NO: 80 - DNA, 120 bp, synthetic construct
  misc_feature 1..120: note = Edit sequence for site O3
  source 1..120: mol_type = other DNA
ccaaggtgaaagcggaagtaattccttcgcgcacctcatggaatcccttctgcagcaccgcaatcgcttttccgagctcacggcggtctcaagcactacctacgtcagcacctgggaccc

SEQ ID NO: 81 - DNA, 53 bp, Homo sapiens
  source 1..53: mol_type = genomic DNA
cagcacctggatcgcttttccgagcttctggcggtctcaagcactacctacgt

SEQ ID NO: 82 - DNA, 53 bp, synthetic construct
  misc_feature 1..53: note = Bottom line of Fig. 14
  source 1..53: mol_type = other DNA
cagcaccgcaatcgcttttccgagctcacggcggtctcggacactacctacgt

SEQ ID NO: 83 - DNA, 61 bp, Homo sapiens
  source 1..61: mol_type = genomic DNA
gggccttcgcgcacctcatggaatcccttctgcagcacctggatcgcttttccgagcttct

SEQ ID NO: 84 - DNA, 61 bp, Homo sapiens; identical to SEQ ID NO: 83
  source 1..61: mol_type = genomic DNA

SEQ ID NO: 85 - DNA, 61 bp, synthetic construct
  misc_feature 1..61: note = Bottom line of Fig. 15
  source 1..61: mol_type = other DNA
attccttcgcgcacctcatggaatcccttctgcagcaccgcaatcgcttttccgagctcac

SEQ ID NO: 86 - DNA, 2031 bp, synthetic construct
  misc_feature 1..2031: note = MMLV_MO1
  source 1..2031: mol_type = other DNA
accctgaacatcgaggacgagtatcggcttcatgagaccagcaaagagccggacgtctccctgggaagcacctggctgagcgacttcccgcaagcctgggcggagaccggtggtatggggctcgcagtgcggcaagcgccgttgataatcccgctaaaggccacgagcacgcccgtgtctatcaagcagtacccgatgagtcaagaggcacgcctcggtatcaagccgcatatccagcgcctcctggaccagggcatcctcgtgccctgccagtctccctggaatacgcctctgctacccgtcaagaagcctggcaccaacgattacaggccggtgcaagacctgcgtgaggtcaacaagcgcgtggaggacatccacccaacggtgcccaacccgtacaatctcctatctggccttccgccctcgcaccagtggtacacggtcttggacctaaaggacgcattcttctgtctgaggctgcaccctacgtcccagccgctgttcgccttcgagtggcgcgacccggagatgggcatctctggccagctaacttggacgcgattgccccaggggtttaagaactcgcccacactcttcaacgaggcactccaccgtgacctggccgactttcgcatacagcaccccgaccttatcctgttgcagtacgtcgatgacctgctcctggcggccacgtccgagctggactgccagcaaggcacccgcgccctacttcaaaccctgggcaacctgggttaccgtgcgtccgccaagaaggcccaaatctgccaaaagcaagtcaagtacctcggctacctcttgaaggagggacagcgctggctgacggaggcgaggaaggagacggtgatgggtcagcccacacccaagaccccgaggcagctaagggagttcctggggaaggcgggcttctgccgtctattcatccctggcttcgcggagatggcggccccgctgtacccgctaacgaagccgggcacgctgttcaactggggccctgaccagcagaaggcgtaccaggagatcaagcaagcgttgcttactgccccagcactcggcctccccgacctcacaaagccgttcgagctattcgttgacgagaaacagggctacgcgaagggtgtgctgactcaaaagctagggccgtggcgacgcccagtagcctacctgagcaagaagctcgaccccgtggcggcgggctggccaccatgcctccggatggtcgcggctatcgcggtgctcacaaaggatgcggggaagctcacgatggggcagcccctggtgatcctggccccacacgcggtggaggcgttagtgaagcaacctcccgaccgatggctgagcaacgcccgcatgacccactaccaggcgctcctcctcgacaccgaccgcgtgcaattcggccctgtcgtggcactgaacccggccacgctgctcccactgcccgaggaggggctacagcacaactgcctcgatatactggcggaagcccacggcacccgccccgacttgacggaccagccgcttcccgacgcggaccatacgtggtacaccgacgggagttccttactccaagagggccagcgaaaggcgggcgctgcggtgaccactgagacggaagtaatctgggcaaaggcgctgcctgcgggcacgtctgcccagcgggcggagctgatcgccctgacccaggccctcaagatggccgagggcaagaagctgaacgtttacaccgacagtcggtatgccttcgcaactgcccacatccacggcgaaatctaccgtcggcgcggctggctgacgagcgagggcaaggagatcaagaacaaggacgagatcctcgccctgctaaaggcactcttcctgcccaagcgactgtccatcattcactgtccggggcaccagaagggccattccgccgaggcgcggggcaaccgcatggcggatcaggccgctcggaaggcggcgatcaccgagacgcccgatacgagcacgctcctgattgaaaactcgtcgccg

SEQ ID NO: 87 - DNA, 2031 bp, synthetic construct
  misc_feature 1..2031: note = MMLV_MO2
  source 1..2031: mol_type = other DNA
accctgaacattgaggacgagtatcgcctgcacgagaccagcaaggagcccgacgtgagcctcggctcaacgtggctcagtgacttcccacaagcctgggccgagactggtggaatggggctggccgtgcgccaagctcccctcatcattcctctcaaggccacttcgacgcctgtctccatcaagcagtaccccatgtcccaggaggctaggctgggcatcaagccgcacatccaacgcctgttagatcaaggaatactggtcccctgccagtcgccgtggaatactccgctactgcctgtcaagaagccgggcacgaacgactaccggcctgtccaagacctgcgcgaggtgaacaagcgggtcgaggacattcaccccaccgttcccaacccttacaacctattgtctggcctcccaccgagccatcagtggtacaccgtcctggacctcaaggatgcgttcttctgtctgcggctacaccctacgtcgcaaccactcttcgccttcgagtggcgggatccggagatggggatctcggggcagctcacgtggactcggctgcctcaggggttcaagaactccccaacactctttaacgaggcactgcatcgggatctggccgacttccgtatccagcacccagacctcatcctcctacagtacgtggacgacttgctgctggccgcgaccagcgagctggactgccagcaagggacacgcgcgctgctccagacgctcgggaacctgggataccgcgccagcgctaagaaggctcaaatctgtcagaaacaagtgaagtacctgggttacctgctcaaggagggtcagcgttggcttaccgaggcccgcaaggagaccgtgatggggcaacccacgccaaagacgccccgacagctacgcgagttcctgggcaaggctgggttctgtcggttgttcatccccggtttcgctgagatggccgcccctctctacccgctgacgaaacctgggactctgttcaactggggccctgaccagcagaaggcgtaccaggagatcaagcaagcgctgcttaccgccccggcgctcggattgccggaccttaccaagcccttcgagctgttcgtggacgagaagcaaggttacgcgaagggcgtcctgacacaaaagttggggccctggcgtaggccggtcgcctacctcagcaagaagttggaccccgtggcggcgggctggccgccgtgcctccgtatggtggcagccatcgccgttctcaccaaagacgctggcaagctcacgatggggcagcctctggtgatcctggcaccccatgccgtcgaggcgctcgtgaagcagccgcctgaccgctggctgagtaacgcacggatgacccattaccaagccctattgctagacacggatcgcgtccaatttgggcccgtggtggctctgaaccctgccactctccttcccctccctgaggagggcttgcagcataactgcctggacatactggctgaggcccacgggacaaggccggacctaacggaccagcctctaccggacgcggatcacacatggtacaccgacggctcctctctcctacaagaggggcagcggaaggcgggtgccgccgtcaccacggagacggaggtgatctgggctaaggcactgcccgccgggacttcggcacagcgagccgagctaatagccctcacacaagcgctgaaaatggccgagggcaagaagctaaacgtctatacggactcccgatacgctttcgccaccgcccacattcatggcgaaatctaccgccgccgtggctggctgacgtccgagggcaaggagatcaagaacaaggacgagatcctcgccctcctgaaagccctgttcctgccgaaaaggctttcgataatccactgccccggccaccagaaggggcactccgccgaggcacgcggcaaccgtatggccgaccaggccgcccggaaggcggcgatcacggaaaccccggacacatccacgctcctcatcgagaacagcagcccc

SEQ ID NO: 88 - DNA, 2031 bp, synthetic construct
  misc_feature 1..2031: note = MMLV_MO3
  source 1..2031: mol_type = other DNA
accctcaacatcgaggacgagtaccgtctgcacgagacgtcgaaggagccggatgtctcactcggctccacgtggctcagcgacttcccacaggcgtgggcggagaccggtggcatgggactggcggtgcgacaagcgccccttatcattcccctaaaggcgacgtcaactcctgtttccattaagcagtaccctatgtcccaggaggcccggctcggcatcaagccacacatccaacggctattggaccagggtatcttggtgccgtgccaatccccgtggaacactccccttctacccgtcaagaagccaggcaccaacgactaccgccccgtgcaagacctgcgcgaggtcaacaagcgagtggaggacatccatcctaccgtcccgaacccgtacaacctgctttccggcctcccgccctcgcaccagtggtacaccgttctcgacttgaaggacgcattcttctgtctgcgcctccacccaacgagccagcccctcttcgctttcgagtggcgcgacccggagatgggaatttcgggccagcttacatggacccgcctcccacaggggttcaagaacagcccgacgctgttcaacgaggccctgcaccgcgacttggcagacttccgaatccagcatcccgacctaatcctcctgcaatacgttgacgacttattgctggccgcgaccagcgagcttgactgccagcaagggactcgcgcgcttctacagacgctcgggaacctgggctaccgtgcctcagctaagaaggcccagatttgccagaagcaagttaagtatctcggctacctcctcaaggagggacagcggtggctgacggaggcccgcaaggagacggtcatgggccagccaacaccgaaaacgcccagacaactccgcgagttcctcggcaaagcgggcttctgtcggctgtttatccccggcttcgccgagatggccgcgcccctctacccactgacgaaacccggcaccctgttcaactggggcccggatcagcagaaggcgtatcaggagatcaagcaagcactcctgacagccccggccctgggattgcccgaccttacgaagcccttcgagttattcgtggacgagaagcaaggctatgcgaagggtgtcctcacgcagaagctggggccctggaggcggcccgtcgcgtacctaagcaagaagctcgacccagtggcggcgggttggccgccctgcctccgcatggtggccgcgattgcggttctcacaaaggacgccgggaagctcacgatggggcagccacttgtcatcctcgctccgcacgccgtggaggcactggtgaagcagccgccggatcgctggctgtctaatgctcgcatgacccactatcaggcgctgctcctagacactgacagggttcagttcggccccgttgtcgcgcttaaccccgctacactactcccgctgccggaggagggtttgcagcataactgcctcgacatcctcgccgaggcccacggcacgcgacccgacctaacggaccagccgctgccggacgctgaccacacttggtacaccgacggcagctccctcctgcaagagggacagcggaaggccggtgccgccgtgacgacggagacggaggtgatatgggctaaggccctgcccgctggtacgtccgcccagcgagccgagctgatcgccctgacgcaagccctcaagatggccgagggcaagaagctaaacgtctatacggacagccgctacgcattcgccacagcccacattcacggagagatataccggaggcgcggctggcttacgtccgaaggcaaggagattaagaacaaagatgagattctggcgctgttgaaggccctcttcctccccaagcggctttccatcatacactgtccaggccaccagaagggccactcggcggaggcgcggggcaaccggatggccgaccaggcggcgcgcaaagccgcgatcacggagaccccagacacttccacgctcctgatcgagaacagtagcccc

SEQ ID NO: 89 - DNA, 2031 bp, synthetic construct
  misc_feature 1..2031: note = MMLV_DO1
  source 1..2031: mol_type = other DNA
actttgaatatcgaagatgagtaccggctacatgagacgtctaaggagcctgatgtttcactcgggagcacttggctaagcgatttcccacaagcgtgggctgaaacgggcgggatgggcctcgctgtaagacaagcgccacttatcatcccgcttaaagctacttctactcccgtctctattaagcaatacccaatgtcccaggaagctcgtttgggcattaagcctcatatacaaaggctactcgatcagggcatacttgttccctgccaatcaccgtggaatacgcccctattaccagttaagaagcctgggactaacgactatcgccccgtacaggatctacgtgaggtgaacaagcgtgtagaggacatccatccgaccgttccgaatccatacaatttgctttctggattacctccaagtcatcaatggtacaccgtgctggatctcaaggacgcattcttctgtctaagattacatcctactagccagccacttttcgcattcgagtggcgagatcccgagatgggaatttcgggccagcttacatggacgaggcttcctcaaggcttcaagaactctcctaccttgttcaatgaggctctacaccgcgacctcgcagacttccggatacaacatccggacctcatactcctacaatatgtggacgatctattgctggccgcgacgagcgaattggattgtcagcaaggaacccgcgccttgttacaaacgttggggaacttggggtatcgagcatcagccaagaaggcacaaatctgccagaaacaagtgaagtatttggggtacttactgaaagaggggcaacgatggttgaccgaagctcgcaaggaaactgttatgggccagccgacacctaagactccaagacagctccgagagttcctcggcaaggctgggttctgtcgcctattcattcctgggtttgccgaaatggctgctcctctgtacccgttgaccaaaccgggaaccttgttcaattggggaccagatcaacagaaggcgtatcaggagatcaagcaagcgctgttgactgcgcctgcgttgggcttgccggatttgacaaaaccctttgaacttttcgttgacgagaaacaaggttacgcgaagggagttcttacacaaaagctgggcccctggagacgacctgttgcttatcttagcaagaagttagatcccgttgctgcggggtggccgccctgcttgaggatggttgccgccattgcggttctgactaaagatgcgggcaaattgacgatgggccagccgctcgtaattctcgccccgcacgcagtcgaagccctagtgaagcagcctcctgaccgttggctctccaacgctcggatgacccactatcaagcgctgctgttggataccgatagagttcaattcgggccggtcgtagcgctcaatcccgcaacgctattacccctgcctgaggagggactacaacataactgcttggatattctagcggaggctcatgggacaagacctgacttgacagatcagcccttgccagatgccgaccacacatggtacactgacggctcatcacttctacaagagggccaaaggaaggccggagctgcggtgacgactgaaaccgaggtgatctgggcaaaggctttacccgctggaacttctgctcagcgcgcagagcttatcgccctaactcaagccctgaaaatggctgagggcaagaagttgaatgtctataccgattcacggtatgctttcgctaccgcccacattcatggagaaatctatcggcgacgaggttggctcacgtctgaggggaaggagattaagaacaaggacgaaatcttagctctgctgaaagctctattcttacccaaacgcttgtcgattatccactgcccaggacaccaaaagggccattccgccgaagccaggggaaaccgtatggccgatcaagctgcccggaaggctgcaataaccgaaacccctgacacttccacgctactcatcgaaaactctagccca

SEQ ID NO: 90 - DNA, 2031 bp, synthetic construct
  misc_feature 1..2031: note = MMLV_DO2
  source 1..2031: mol_type = other DNA
accttgaatatcgaagatgagtacagactccatgagaccagcaaggagcctgatgtgagcctgggaagtacatggctatctgactttcctcaggcttgggcggagaccggtggcatgggcctggctgtccggcaagcacccctaatcatcccgttgaaagcaacgtccacacccgtatctatcaagcagtacccgatgagccaagaggcacggctagggattaagccgcacatccaaagattgttagaccaggggatactcgttccctgtcaatctccgtggaacacaccgttactgcctgttaagaagcccggcacaaatgattatcggcctgtacaagacctgcgcgaagtgaacaaacgagtggaagatattcaccccaccgtgccgaatccttacaacttgctaagcggtttaccacccagtcaccagtggtacaccgtcttggacctcaaagacgcattcttctgcttgcggttgcatcctacgagtcagccgctctttgcctttgaatggagagatcccgagatgggcataagtggtcagcttacatggacgaggctacctcagggattcaagaactcgcctaccttattcaatgaggctctacacagggatcttgctgactttcggatacaacaccctgacttaattctgctacaatatgttgacgatctgctgcttgcggcgaccagtgaactcgactgccaacaagggacaagagccttgttacagacacttggcaatctcggctaccgcgcgtctgctaagaaggcacagatttgccaaaagcaagtgaagtatctcggttacctgctgaaagagggacaaagatggttgaccgaagccaggaaggaaactgtcatggggcaacccacccctaaaaccccaagacagcttagagagttcctcgggaaagctgggttctgccgcctgttcattcccggtttcgctgaaatggctgcacctctatacccactgaccaaaccgggcacgctattcaattggggaccggatcaacaaaaggcttaccaggagatcaagcaagctctattgacagctccggctctgggtttacccgacttgactaaacccttcgagttgttcgttgacgagaaacagggctacgcgaaaggcgtactgacgcagaaactgggcccgtggcggcgacccgttgcttacctttccaagaagctggaccctgttgccgctggctggccgccctgccttcgcatggttgcggccattgctgtccttacgaaagatgctgggaaactaactatgggtcagccgctcgtaattctcgcgccacacgctgtcgaggctctagtcaaacaacctcctgaccgctggctgtcgaatgcacggatgacacattaccaggcgttgctattggacactgaccgagtacaatttggaccagtagtggctttaaatcccgcgacactccttccccttcctgaggagggtttgcaacacaactgtcttgatatacttgctgaggcccacggaacaagacccgatctcacagatcaacccctccccgacgcagatcacacctggtacaccgacggttcaagcctgttacaggaaggccagcgaaaggccggagcagccgtgacaaccgaaaccgaagttatctgggcaaaggccctaccagccgggacaagcgcgcagagggccgagctgatcgcgctcacacaagcactcaaaatggccgaaggcaagaagctcaatgtttacactgattcccgctacgcattcgctaccgctcatattcacggagaaatctacaggaggcgagggtggcttactagcgaaggaaaggagattaagaacaaagacgagatcctggcactattgaaagccttgttcctgccaaagcgcttatctatcattcactgtccgggccaccagaagggccacagtgctgaggctcggggcaatcggatggcagatcaggccgcacgaaaagccgcaattactgagacacctgacacatctaccttgctgattgagaactcgtctccg

SEQ ID NO: 91 - DNA, 2073 bp, Soybean chlorotic mottle virus
  source 1..2073: mol_type = genomic DNA
aatactgaaattgtccaaaaacaccgagttttaaccaaaggtaaccctaatgttactttcataaaagttagtataggcaaaagaaatttcttggcttatattgatactggagcaactctgtgctttggaaaaagaaaaatttcaaataattgggaaattttaaaacaaccaaaagaaattatcattgcagataaatcaaaacactatattagagaagctatttctaatgtgtttttaaaaatcgaaaataaagaattcttaatccctatcatatatttacatgattcaggattagatttaattataggaaacaatttcctaaaattataccaaccttttattcagagattggaaacaattgaattaagatggaaaaatcttaataacccaaaagaatctcaaatgatttcaaccaagattcttacaaaaaatgaagtattaaaactttcatttgaaaaaattcatatttgtttagaaaaatatttatttttcaaaacaattgaagaacaactcgaagaagtatgttcagaacatccactggatgaaacaaaaaataaaaatggtcttttaatagaaataagacttaaagacccattacaagaaataaatgtcacaaatagaattccatatacaataagagatgtacaagaattcaaggaggaatgtgaagacctcttaaaaaagggcttaattcgagaatctcaaagtccacacagtgcaccggcattctatgtcgaaaatcacaatgaaatcaagcgtggaaaaagacgcatggtaattaattacaaaaaaatgaatgaagccacaattggcgattcatataagttaccaagaaaagattttattctggaaaaaataaaaggatctttatggttttcaagcttggatgctaaatctggatactaccagctaaggctccatgaaaatacaaagcctctaacagctttttcatgtccacctcagaaacattacgaatggaatgttttaagttttggacttaaacaagcaccatctatatatcaaagatttatggatcaatccctcaagggacttgaacatatatgtttggcatatattgatgacatcctgatctttacaaaaggatctaaagaacaacatgtaaatgatgttcggattgttttgcaaagaatcaaagaaaaaggaattattatttctaagaaaaaatcaaaactgattcaacaggaaatcgaatatctcggtttaaaaatacaagggaatggagaaattgatttatcacctcatacccaagaaaaaattcttcaatttcctgatgaattagaagatagaaaacaaatacagcgttttcttggctgtattaattacattgcaaatgaaggatttttcaaaaatcttgctctagaaagaaagcaccttcaaaagaaaatttctgttaaaaacccctggaaatgggatacaatagatacaaaaatggttcagtccataaaaggcaaaattcaaagcctaccaaaattatataatgcatcgattcaagactttttaatagtcgagacagatgcatcgcaacactcctggagtggatgtttgcgagctttacccaagggaaagcaaaaaatcggactcgatgaattcgggataccgacagctgacctctgcacaggtagcagttcagcttcaagcgataattcgccagctgagattgacaaatgtcattcagccagtaaacaggacactcatgtggccagtaaaataaagaaactcgaaaacgagcttctactttgcaaatatgtttcaggtaccttcacagatacggaaacaagataccctatagcagaactggaggttcttgctggagtaaaagtcctagaaaaatggagaatcgacctcctacaaacgaggttcctcctccgcactgacagcaagtactttgcaggtttttgtaggtacaacatcaagacagactaccggaacggacgtctaatcaggtggcaactacggttacaagcctatcaaccgtacgtggaattaatcaaatcagaaaataacccattcgcagatacgcttacgcgagaatggagcaagccatcaagcagt

SEQ ID NO: 92 - DNA, 2073 bp, synthetic construct
  misc_feature 1..2073: note = SbCMV_DO1
  source 1..2073: mol_type = other DNA
aacactgagattgttcaaaaacatagagtgcttaccaaaggaaatccaaatgttacatttataaaagtgtccatagggaagagaaattttttagcttatattgacactggagccacactctgttttggaaaaaggaaaatatcaaataactgggaaatccttaagcaacccaaagaaatcattatcgctgataagtcaaaacactacatcagagaagctataagtaacgtattcctgaaaattgaaaacaaggagttcttgatacccattatatatcttcatgattcagggttggatttgattattgggaataacttcctgaagctttatcaaccatttattcaaagacttgaaactatcgaactcaggtggaaaaacttgaacaatcccaaagagtctcaaatgattagcactaaaattcttacgaaaaatgaagttcttaagctgagttttgagaagattcatatttgtctcgaaaaataccttttctttaaaaccatcgaggaacaacttgaggaggtttgttctgaacatccacttgatgagacaaagaacaagaatggtcttttgattgagatacgtctgaaagatcctctgcaggagattaacgtcacaaataggattccatataccattagagatgtacaggaattcaaggaagaatgtgaagatttacttaagaagggtctcattcgtgaatcacaatctccccacagtgcacccgcattttacgttgaaaatcataatgaaattaagagaggcaagcgtagaatggttatcaactacaagaagatgaatgaagcaaccataggagatagctacaaactcccgcgaaaggattttatcttagagaagataaagggcagtttgtggttttcaagtttagatgcaaaatcaggttattatcagcttcgcttacatgagaacacaaagcctctcactgctttctcttgccctcctcaaaaacattatgaatggaatgtgttgagtttcggtctaaaacaggcaccttcgatttaccagcgcttcatggaccagtccttaaagggattagagcacatttgcttggcatatatagatgatatcttaatctttactaaaggctcaaaggaacagcatgtcaatgatgttcggattgtcctgcaaagaataaaagagaaaggaatcataatatctaaaaaaaaatcaaaattgattcagcaagagattgaatatctaggattgaaaattcaaggtaatggtgaaattgacctctcaccacatactcaagaaaagatcctacagttccctgatgaactggaggatagaaaacaaatacagaggtttctaggttgcattaattacattgcgaacgaaggatttttcaaaaatcttgccctagagagaaagcacttgcagaagaagatttccgtgaagaatccatggaagtgggatacaatagacacaaaaatggtgcaatcaatcaagggcaaaattcaatccctgccgaagctctacaatgcaagtattcaggatttcctaattgtagagactgacgcctcgcaacattcttggtctgggtgtttgcgggctcttccaaagggcaagcagaaaatcggtctggacgaatttgggattccaacggcagatttatgtactggtagctccagtgcttcctctgataattctcctgctgagatcgacaagtgccactcagcctcgaagcaggatacacacgtcgcctctaaaataaagaaacttgagaatgagttacttttgtgcaagtatgtttcagggactttcacggacaccgagactaggtatcctatagctgaactcgaggtgttggcgggtgttaaagttttggaaaaatggaggatagacttgttgcaaacacgatttctacttaggacagattccaaatattttgctggattttgtagatacaacattaagactgattatcggaacgggaggctcataagatggcaattgcgccttcaagcttaccagccttatgtggaactgatcaagagtgaaaataatccttttgcagacaccctaacacgagagtggagcaaaccatcttctagc

SEQ ID NO: 93 - DNA, 2034 bp, Cauliflower mosaic virus
  source 1..2034: mol_type = genomic DNA
gatcatctacttctgaagactcagactcagactgagcaggtgatgaacgtcaccaatcccaattcgatctacatcaagggaagactctacttcaagggatacaagaagatagaacttcactgtttcgtagacacgggagcaagcctatgcatagcatccaagttcgtcataccagaagaacattgggtcaatgcagaaagaccaattatggtcaaaatagcagatggaagctcaatcaccatcagcaaagtctgcaaagacatagacttgatcatagccggcgagatattcagaattcccaccgtctatcagcaagaaagtggcatcgatttcattatcggcaacaacttctgtcagctgtatgaaccattcatacagtttacggatagagttatcttcacaaagaacaagtcttatcctgttcatattgcgaagctaaccagagcagtgcgagtaggcaccgaaggatttcttgaatcaatgaagaaacgttcaaaaactcaacaaccagagccagtgaacatttctacaaacaagatagaaaatccactagaagaaattgctattctttcagaggggaggaggttatcagaagaaaaactctttatcactcaacaaagaatgcaaaaaatcgaagaactacttgagaaagtatgttcagaaaatccattagatcctaacaagactaagcaatggatgaaagcttctatcaagctcagcgacccaagcaaagctatcaaggttaaacccatgaagtatagcccaatggatcgcgaagaatttgacaagcaaatcaaagaattactggacctaaaagtcatcaagcccagtaaaagccctcacatggcaccagccttcttggtcaacaatgaagccgagaagcgaagaggaaagaaacgtatggtagtcaactacaaagctatgaacaaagctactgtaggagatgcctacaatcttcccaacaaagacgagttacttacactcattcgaggaaagaagatcttctcttccttcgactgtaagtcaggattctggcaagttctgctagatcaagaatcaagacctctaacggcattcacatgtccacaaggtcactacgaatggaatgtggtccctttcggcttaaagcaagctccatccatattccaaagacacatggacgaagcatttcgtgtgttcagaaagttctgttgcgtttatgtcgacgacattctcgtattcagtaacaacgaagaagatcatctacttcacgtagcaatgatcttacaaaagtgtaatcaacatggaattatcctttccaagaagaaagcacaactcttcaagaagaagataaacttccttggtctagaaatagatgaaggaacacataagcctcaaggacatatcttggaacacatcaacaagttccccgatacccttgaagacaagaagcaacttcagagattcttaggcatactaacatatgcctcggattacatcccgaagctagctcaaatcagaaagcctctgcaagccaagcttaaagaaaacgttccatggagatggacaaaagaggataccctctacatgcaaaaggtgaagaaaaatctgcaaggatttcctccactacatcatcccttaccagaggagaagctgatcatcgagaccgatgcatcagacgactactggggaggtatgttaaaagctatcaaaattaacgaaggtactaatactgagttaatttgcagatacgcatctggaagctttaaagctgcagaaaagaattaccacagcaatgacaaagagacattggcggtaataaatactataaagaaatttagtatttatctaactcctgttcattttctgattaggacagataatactcatttcaagagtttcgttaatctcaattacaaaggagattcgaaacttggaagaaacatcagatggcaagcatggcttagccactattcatttgatgttgaacacattaaaggaaccgacaaccactttgcggacttcctttcaagagaattcaataaggttaattcc

SEQ ID NO: 94 - DNA, 2034 bp, synthetic construct
  misc_feature 1..2034: note = CaMV_DO1
  source 1..2034: mol_type = other DNA
gatcaccttctgcttaagacacagacacaaactgaacaagttatgaatgtgaccaacccgaattccatttacattaagggacgactctactttaaggggtataagaaaatagaattacattgtttcgtcgacactggagcatctctctgcatagcgtccaagtttgttattcctgaggaacattgggtaaatgcagaaaggccgattatggttaaaattgcagatggtagcagcatcacaatctcaaaagtttgcaaggacattgacttgatcattgctggtgagattttcagaattcctacagtgtatcaacaagaatccggcattgattttataattggtaataacttttgtcaactttatgagcccttcatacaatttacagatcgagtcatttttactaaaaacaagagttaccctgttcacattgcaaaactcactcgtgccgtgagagttggaacggaaggatttctagaatctatgaagaagagatcgaaaactcagcaaccagaacccgttaatatttctacaaataagattgaaaatccattagaggaaatagccatcttgtccgaaggccggcggttgagtgaagaaaagttgtttatcacgcagcagagaatgcaaaaaatagaggagcttctcgaaaaggtttgttctgagaatcctttggatccaaataaaacaaaacaatggatgaaagctagtataaagctttcagacccatcaaaggcaattaaggtgaagccaatgaaatatagccccatggatagggaggagtttgacaagcaaattaaggagctactcgatctgaaagtaataaaaccttctaaatcgcctcacatggctccagcattcctggttaacaacgaggctgaaaagcgcagaggaaaaaaaagaatggtggtgaactacaaagcaatgaataaggctactgttggagatgcttataatcttcctaataaagatgagctcttgaccttaattagagggaagaaaattttctcctcatttgattgtaaatcaggattttggcaagtgttgctggatcaagagtctcgtccactgaccgcctttacgtgccctcaaggacattatgaatggaatgtcgtaccatttggtctcaagcaagcaccttctattttccagaggcatatggatgaagcatttagagtgtttaggaaattctgctgtgtttatgtggatgatatattggtattctcaaataatgaggaagaccatttgctgcatgttgccatgattcttcagaagtgcaatcaacatggaatcatcttatccaagaagaaggctcagttgttcaagaagaagataaattttttgggtctcgagattgatgaggggacacataagcctcagggtcatatactagaacatatcaacaagtttccagacactttggaagacaaaaagcagttgcaaaggttccttgggattctgacttatgcttcagattatataccaaagcttgctcaaataagaaaaccccttcaggcgaagctcaaagaaaacgttccttggaggtggactaaggaggataccttatacatgcagaaagtcaagaaaaacctccagggtttcccaccgctccaccatcctttacctgaagaaaaactaattatcgagacagatgcttctgatgactactggggcggcatgttgaaggccatcaaaatcaatgaagggaccaatactgagctcatttgtcgatatgcaagcggaagttttaaagcagctgagaaaaattatcatagtaatgataaagagactctagccgttattaacaccataaagaaattctctatatatcttacccccgtccactttttaatcaggacagacaacactcacttcaaatcatttgtgaacctgaattacaagggtgatagtaaacttggccgtaacatacgctggcaggcttggttgagccactactcttttgatgtagaacacattaaaggaacagataatcattttgctgatttcctttctcgcgagttcaacaaagtaaattca

SEQ ID NO: 95 - DNA, 117 bp, synthetic construct
  misc_feature 1..117: note = pegRNA
  source 1..117: mol_type = other DNA
agatgaaaccaaaagaagaggttttagagctagaaatagcaagttaaaataaggctagtccgttatcaacttgaaaaagtggcaccgagtcggtgcatggcaatggttcttttggtt

SEQ ID NO: 96 - DNA, 117 bp, synthetic construct
  misc_feature 1..117: note = pegRNA
  source 1..117: mol_type = other DNA
cacttcctatgcacaatggagttttagagctagaaatagcaagttaaaataaggctagtccgttatcaacttgaaaaagtggcaccgagtcggtgctgagtctggcattgtgcatag

SEQ ID NO: 97 - DNA, 117 bp, synthetic construct
  misc_feature 1..117: note = pegRNA
  source 1..117: mol_type = other DNA
ttggtagtagcgactccatggttttagagctagaaatagcaagttaaaataaggctagtccgttatcaacttgaaaaagtggcaccgagtcggtgcttaacttatgggagtcgctac

SEQ ID NO: 98 - DNA, 117 bp, synthetic construct
  misc_feature 1..117: note = pegRNA
  source 1..117: mol_type = other DNA
gctcttcctgcgccattaaagttttagagctagaaatagcaagttaaaataaggctagtccgttatcaacttgaaaaagtggcaccgagtcggtgctaagtacttaaatggcgcagg

SEQ ID NO: 99 - DNA, 117 bp, synthetic construct
  misc_feature 1..117: note = pegRNA
  source 1..117: mol_type = other DNA
gccgttaatttgagagtccagttttagagctagaaatagcaagttaaaataaggctagtccgttatcaacttgaaaaagtggcaccgagtcggtgcagctgaattaactctcaaatt

SEQ ID NO: 100 - DNA, 117 bp, synthetic construct
  misc_feature 1..117: note = pegRNA
  source 1..117: mol_type = other DNA
gagattgttattgctggtgcgttttagagctagaaatagcaagttaaaataaggctagtccgttatcaacttgaaaaagtggcaccgagtcggtgcgaaaaaatcacagcaataaca

SEQ ID NO: 101 - DNA, 117 bp, synthetic construct
  misc_feature 1..117: note = pegRNA
  source 1..117: mol_type = other DNA
gaggcaagagatgtcctagggttttagagctagaaatagcaagttaaaataaggctagtccgttatcaacttgaaaaagtggcaccgagtcggtgccttcacctttaggacatctct

SEQ ID NO: 102 - DNA, 20 bp, synthetic construct
  misc_feature 1..20: note = spacer
  source 1..20: mol_type = other DNA
agatgaaaccaaaagaagag

SEQ ID NO: 103 - DNA, 20 bp, synthetic construct
  misc_feature 1..20: note = spacer
  source 1..20: mol_type = other DNA
cacttcctatgcacaatgga

SEQ ID NO: 104 - DNA, 20 bp, synthetic construct
  misc_feature 1..20: note = spacer
  source 1..20: mol_type = other DNA
ttggtagtagcgactccatg

SEQ ID NO: 105 - DNA, 20 bp, synthetic construct
  misc_feature 1..20: note = spacer
  source 1..20: mol_type = other DNA
gctcttcctgcgccattaaa

SEQ ID NO: 106 - DNA, 20 bp, synthetic construct
  misc_feature 1..20: note = spacer
  source 1..20: mol_type = other DNA
gccgttaatttgagagtcca

SEQ ID NO: 107 - DNA, 20 bp, synthetic construct
  misc_feature 1..20: note = spacer
  source 1..20: mol_type = other DNA
gagattgttattgctggtgc

SEQ ID NO: 108 - DNA, 20 bp, synthetic construct
  misc_feature 1..20: note = spacer
  source 1..20: mol_type = other DNA
gaggcaagagatgtcctagg

SEQ ID NO: 109 - DNA, 10 bp, synthetic construct
  misc_feature 1..10: note = reverse transcriptase target sequence
  source 1..10: mol_type = other DNA
atggcaatgg

SEQ ID NO: 110 - DNA, 10 bp, synthetic construct
  misc_feature 1..10: note = reverse transcriptase target sequence
  source 1..10: mol_type = other DNA
ttaacttatg

SEQ ID NO: 111 - DNA, 10 bp, synthetic construct
  misc_feature 1..10: note = reverse transcriptase target sequence
  source 1..10: mol_type = other DNA
taagtactta

SEQ ID NO: 112 - DNA, 10 bp, synthetic construct
  misc_feature 1..10: note = reverse transcriptase target sequence
  source 1..10: mol_type = other DNA
agctgaatta

SEQ ID NO: 113 - DNA, 10 bp, synthetic construct
  misc_feature 1..10: note = reverse transcriptase target sequence
  source 1..10: mol_type = other DNA
gaaaaaatca

SEQ ID NO: 114 - DNA, 10 bp, synthetic construct
  misc_feature 1..10: note = reverse transcriptase target sequence
  source 1..10: mol_type = other DNA
cttcaccttt

SEQ ID NO: 115 - DNA, 11 bp, synthetic construct
  misc_feature 1..11: note = primer binding site sequence
  source 1..11: mol_type = other DNA
ttcttttggtt

SEQ ID NO: 116 - DNA, 11 bp, synthetic construct
  misc_feature 1..11: note = primer binding site sequence
  source 1..11: mol_type = other DNA
attgtgcatag

SEQ ID NO: 117 - DNA, 11 bp, synthetic construct
  misc_feature 1..11: note = primer binding site sequence
  source 1..11: mol_type = other DNA
ggagtcgctac

SEQ ID NO: 118 - DNA, 11 bp, synthetic construct
  misc_feature 1..11: note = primer binding site sequence
  source 1..11: mol_type = other DNA
aatggcgcagg

SEQ ID NO: 119 - DNA, 11 bp, synthetic construct
  misc_feature 1..11: note = primer binding site sequence
  source 1..11: mol_type = other DNA
actctcaaatt

SEQ ID NO: 120 - DNA, 11 bp, synthetic construct
  misc_feature 1..11: note = primer binding site sequence
  source 1..11: mol_type = other DNA
cagcaataaca

SEQ ID NO: 121 - DNA, 11 bp, synthetic construct
  misc_feature 1..11: note = primer binding site sequence
  source 1..11: mol_type = other DNA
aggacatctct

SEQ ID NO: 122 - DNA, 20 bp, synthetic construct
  misc_feature 1..20: note = nicking spacer sequence
  source 1..20: mol_type = other DNA
ctggcccctccattgtgcat

SEQ ID NO: 123 - DNA, 20 bp, synthetic construct
  misc_feature 1..20: note = nicking spacer sequence
  source 1..20: mol_type = other DNA
taattatcattataattctt

SEQ ID NO: 124 - DNA, 20 bp, synthetic construct
  misc_feature 1..20: note = nicking spacer sequence
  source 1..20: mol_type = other DNA
cattcaaaacaaacctttaa

SEQ ID NO: 125 - DNA, 20 bp, synthetic construct
  misc_feature 1..20: note = nicking spacer sequence
  source 1..20: mol_type = other DNA
tttgcataatcaacgctgaa

SEQ ID NO: 126 - DNA, 20 bp, synthetic construct
  misc_feature 1..20: note = nicking spacer sequence
  source 1..20: mol_type = other DNA
aatccttaacttatgcccca

SEQ ID NO: 127 - DNA, 20 bp, synthetic construct
  misc_feature 1..20: note = nicking spacer sequence
  source 1..20: mol_type = other DNA
atgaaaactacaaatataga

SEQ ID NO: 128 - DNA, 20 bp, synthetic construct
  misc_feature 1..20: note = nicking spacer sequence
  source 1..20: mol_type = other DNA
gggaaggacacaaaagaaaa

SEQ ID NO: 129 - DNA, 76 bp, synthetic construct
  misc_feature 1..76: note = sgRNA scaffold
  source 1..76: mol_type = other DNA
gttttagagctagaaatagcaagttaaaataaggctagtccgttatcaacttgaaaaagtggcaccgagtcggtgc

SEQ ID NO: 130 - DNA, 96 bp, synthetic construct
  misc_feature 1..96: note = sgRNA sequence
  source 1..96: mol_type = other DNA
ctggcccctccattgtgcatgttttagagctagaaatagcaagttaaaataaggctagtccgttatcaacttgaaaaagtggcaccgagtcggtgc

SEQ ID NO: 131 - DNA, 96 bp, synthetic construct
  misc_feature 1..96: note = sgRNA sequence
  source 1..96: mol_type = other DNA
taattatcattataattcttgttttagagctagaaatagcaagttaaaataaggctagtccgttatcaacttgaaaaagtggcaccgagtcggtgc

SEQ ID NO: 132 - DNA, 96 bp, synthetic construct
  misc_feature 1..96: note = sgRNA sequence
  source 1..96: mol_type = other DNA
cattcaaaacaaacctttaagttttagagctagaaatagcaagttaaaataaggctagtccgttatcaacttgaaaaagtggcaccgagtcggtgc

SEQ ID NO: 133 - DNA, 96 bp, synthetic construct
  misc_feature 1..96: note = sgRNA sequence
  source 1..96: mol_type = other DNA
tttgcataatcaacgctgaagttttagagctagaaatagcaagttaaaataaggctagtccgttatcaacttgaaaaagtggcaccgagtcggtgc

SEQ ID NO: 134 - DNA, 96 bp, synthetic construct
  misc_feature 1..96: note = sgRNA sequence
  source 1..96: mol_type = other DNA
aatccttaacttatgccccagttttagagctagaaatagcaagttaaaataaggctagtccgttatcaacttgaaaaagtggcaccgagtcggtgc

SEQ ID NO: 135 - DNA, 96 bp, synthetic construct
  misc_feature 1..96: note = sgRNA sequence
  source 1..96: mol_type = other DNA
atgaaaactacaaatatagagttttagagctagaaatagcaagttaaaataaggctagtccgttatcaacttgaaaaagtggcaccgagtcggtgc

SEQ ID NO: 136 - DNA, 96 bp, synthetic construct
  misc_feature 1..96: note = sgRNA sequence
  source 1..96: mol_type = other DNA
gggaaggacacaaaagaaaagttttagagctagaaatagcaagttaaaataaggctagtccgttatcaacttgaaaaagtggcaccgagtcggtgc

SEQ ID NO: 137 - DNA, 20 bp, synthetic construct
  misc_feature 1..20: note = spacer
  source 1..20: mol_type = other DNA
ctggcccctccattgtgcat

SEQ ID NO: 138 - DNA, 20 bp, synthetic construct
  misc_feature 1..20: note = spacer
  source 1..20: mol_type = other DNA
taattatcattataattctt

SEQ ID NO: 139 - DNA, 20 bp, synthetic construct
  misc_feature 1..20: note = spacer
  source 1..20: mol_type = other DNA
cattcaaaacaaacctttaa

SEQ ID NO: 140 - DNA, 20 bp, synthetic construct
  misc_feature 1..20: note = spacer
  source 1..20: mol_type = other DNA
tttgcataatcaacgctgaa

SEQ ID NO: 141 - DNA, 20 bp, synthetic construct
  misc_feature 1..20: note = spacer
  source 1..20: mol_type = other DNA
aatccttaacttatgcccca

SEQ ID NO: 142 - DNA, 20 bp, synthetic construct
  misc_feature 1..20: note = spacer
  source 1..20: mol_type = other DNA
atgaaaactacaaatataga

SEQ ID NO: 143 - DNA, 20 bp, synthetic construct
  misc_feature 1..20: note = spacer
  source 1..20: mol_type = other DNA
gggaaggacacaaaagaaaa

SEQ ID NO: 144 - DNA, 18668 bp, synthetic construct
  misc_feature 1..18668: note = pWISE2780
  source 1..18668: mol_type = other DNA
aaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtagacctcaattgcgagctttctaatttcaaactattcgggcctaacttttggtgtgatgatgctgactggcaggatatataccgttgtaatttgagctcgtgtgaataagtcgctgtgtatgtttgtttgattgtttctgttggagtgcagcccatttcaccggacaagtcggctagattgatttagccctgatgaactgccgaggggaagccatcttgagcgcggaatgggaatggatttcgttgtacaacgagacgacagaacacccacgggaccgagcttcgaagctttaacgcttgagttaagccgcgccgcgaagcggcgtcggcttgaacgaattgttagacatcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggctttgtttctccttcaatcattgactattgtcacgttattctgaatcgacgagaactcacatcaatctcaacaagcatctctgctcgcgaaattggtgttgtctcattaaaacgtgaccgtcaaagtctattcactaggacccaaatctctccaggtcccgcaagctagcttcgccgcccacgtttctctttcttctcatattcaattgtcaaaaaacagacctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactttttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacaggctctccttcacgatcaacgatcggcatggggccttcgtgcttgttgagtaatgttatcgctcccatcagagcacgcttggtactccgggaatcggatggtctgtcgatcatccaaaaaacgctcatgttttcaacctattaggtctgtggtcagctgaccacagaccatcctgctccatactcgctaattctagccaaaccgcaacgtcccctgcccgctagccttcaagagcgccattatcatcgggccaagtgaaaacttcccgagctcgctccgccgtgtcagatctcggagatagcccccgggcgaattgatgaagttcgctcgctccaaaatgcacgccatcgctgctgccgcattctccggtcccattgcctcacacgcgtcttggtaagccgacgggctgacccccagcatagaccgaaccaccaccgcagccgacatgaggtcacgccagctagcaaccgcaccgctcggcccataattgccaatggtcgggcatgctttcaggatcatcccgagggggaacgcttttatcggctcgctccttgcccggtctatttcactcggcttagcgccctgctccttttcagagcgaggttcaagttcattaacggattcgggttttgaattctgtatgtgctgctcgctctgggcagcattggtgctattattttctgaattgtctctaatttccaaccggttgattatctcttcctggagcatccacatctcttcgagaattgactctacatcagcaagcgtcggggcgcgtggaattctacccacaagttccacatagacttcctcgacagcttgccagtcgccctccgctccctcttccatagctgccgtaattagcttccgaacgtcccgtcggcaaatcgtcagactttctttggccatcctgaatgctgctcgatcggccatcacctgctgtgccatcatcgctagctcttcggaccgcgcgagaagcggagacaaatcgaagccaaacgcgcgctcgatctgaccagcgccatccttacgagcgtaacgctttccgttggcgctatccttccggacgatcaagcctgactccacgagcatggcgatgtgcctacgcaaagtcgcgccagccatcccatgcgcccgaagggcaagctgagcattcgacgggaagacgatcagctgtgcctcctgacgcaactccgtttccgggtgaaagctcaatagcgcatcaaggacggcaagactgttggactggattccaagtagttccatggccgcggacgcgtctctaaagaccttccacttgtccgctgtcttgccttgtttgatatcggccagcgccgtctggcgccgcacaagcgcaagcgtcattggccgccgcccgaatggcgtcgttacacttcctgtctgcatcatctttcacctttcagcaggcaaaggaaatcagctcaccaaaacggcgctaaaaactcttgacgaggattcgaggaaatgcgattctgttcgcgctagagagacagaagggcttccgcgacggcgacgttgagggggctcttttcttttgcggtttactctccccgtttccgttggttctcagcgtggtacgcttgatacagcgctggcacatgatcgagcacgaaggtcgcaaaatcgggcgtcgccttcctgtcaatcgtgatttccagtttggccttgctctgcgtcacctgtgcaattctggtgccgtctggggtggccatgacctcgggaagtccacgcgcaacccgactgggcttcagactagcgatcaccgccttgaatcgttctgccgatggcagcgcttgaacttcctccgacatagcatatttagccacgtcggccggtgaagaaactttctcaatcagctcggcaagttgttgccaactcggccgtccaacaccaggagcggcaccaatagcatcggtcagttcagaggggagggcgtcgacgagcagaagcatcttggacaaattgctcttgtcgatcgacatcgcggcgatgacaatctctcgagaaaactgcctgttcaggcgatgtgcgaagcgcgccttttcgatgaaggtaagatcttcgcgctcattgttttcctgaccctgtgctacgaccacttgctcgtccgtcagttcgcgaacgaccgctctgaccggaagtccgagttctgaaacggcgcgtagccggcggtggccgaaggcaacctgatatcggcccggctggctcggatgcggtcgcacaaggattgggacttgctgtccttgttcccggatcgaagtaaggagcccgtcaatgtcccctcgcatacgatcctgcacgaaagacggttctattgacgaggcatccaactctatcactgcctgaccttcagcgagacgccgctcgatctcttcggcacggctaagacgatcgttttgctctcgcagtgcgttaccaatgttcgctgtgagcttcgttgccggatcgcgctccttccttgttacgccgaggagcggcatggagcggttctttgccgtcctattgtcggcgggcgacgtctcaggggcgtcagttgagacgccaaggatgtgcttccggctcatgtgggcctaccccatgcttttttgatcagtgtttcgatctcgtcgttgacggcgttcatcgcctccaaggctcgatcataggtcgagcgcgtgaacaggccacgctccacttcgaatagagtctggtttgtcaggccagcgtccgaaaccgcggtggttttaagcatcggaaaattgaggacattttcgccaaaaatcgaccgcagataacctaccatttggttctgtggtccgtcgctcggttcgaaacgggttatcagatagcgcatccaattaaacttgaacttggcgccagcattctcgatttcacgcaaaaggttcgatgtcattgccagaaactggttcatcgacatcacatccagcatctgcggatggaccgtgacaagaatggacgtcgccgcagtcaatgcggatagcgtgagatacccaagctggggagggcagtcgatgaccacgacgtcatagttatccgcgatatcttcaattacttggctgatgcgaccataaaagagcgtgtcgccctctttgcggttcatcagcgcgcgtggcgtatcgtgttcaaactccatcagctcaaggttaccaggaatcaggtggaggtcgggaatgtaagtccctcggacgactcgttcgattgccacctgctcatcatcataccttatagcgccgtagagcgtttcgttcgggccaacgtccgtctccggttggctcccaaagagtgcagaaaggctcgcttgaggatcgagatcaatggccaagactcgatatccgcgcatagcgaggtactgcgccagatgcgcggcggtggtggtcttacccgacccacctttgaaattcatcacagagataacctgaagctgctcgccgcctcgacgatgtggcaggtagcgccggttcccgcggccgacctgatccatatacttccgaatcacatggatatcttcaattgagaacattcgcctgccacccgggctcatgctaacattcaactctggcatctcagacgcggtctgccgtaaatatgactcgccaacgccgagcagcttggacgcctccgatggcccgaatgttcgaatgcccttctcggaatgcggcgggaaaaccttaagatgatgtgcttgaagttggctcgagagggcatcggcatgacgctccatcaaggccgtcaaccctacaactacaggcgctgcttttaggacagacttcgccatctcaaacccattccttgccagtggcgatatttttcgcgaaactggaaaagttccgccgctggcaattagcgccgattctgctgtttgggcaagagcttttaggttaacagaaggttaacgccctcaggtcgaaaaactccacccaactgttatttgtatttatttccaatgccttagagagattgccatttgaatatgttcatgtattgttttagtgataatcctacaatcgtaacccaaaaagaggtcgccctctgcgcgccgtcgtccaatataggcgaagtcacccttgcgactcaggcggattctaccttgtacacgtgtcgagtggaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgcggccctaggcgtacgataacttcgtataatgtatgctatacgaagttatcactagtcaacaattggccaatctttgttctaaattgctaataaacgaccatttccgtcaattctccttggttgcaacagtctacccgtcaaatgtttactaatttataagtgtgaagtttgaattatgaaagacgaaatcgtattaaaaattcacaagaataaacaactccatagattttcaaaaaaacagtcacgagaaaaaaaccacagtccgtttgtctgctcttctagtttttattatttttctattaatagttttttgttatttcgagaataaaatttgaacgatgtccgaaccacaaaagccgagccgataaatcctaagccgagcctaactttagccgtaaccatcagtcacggctcccgggctaattcatttgaaccgaatcataatcaacggtttagatcaaactcaaaacaatctaacggcaacatagacgcgtcggtgagctaaaaagagtgtgaaagccaggtcaccatagcattgtctctcccagattttttatttgggaaataatagaagaaatagaaaaaaataaaagagtgagaaaaatcgtagagctatatattcgcacatgtactcgtttcgctttccttagtgttagctgctgccgctgttgtttctcctccatttctctatctttctctctcgctgcttctcgaatcttctgtatcatcttcttcttcttcaaggtgagtctctagatccgttcgcttgattttgctgctcgttagtcgttattgttgattctctatgccgatttcgctagatctgtttagcatgcgttgtggttttatgagaaaatctttgttttgggggttgcttgttatgtgattcgatccgtgcttgttggatcgatctgagctaattcttaaggtttatgtgttagatctatggagtttgaggattcttctcgcttctgtcgatctctcgctgttatttttgtttttttcagtgaagtgaagttgtttagttcgaaatgacttcgtgtatgctcgattgatctggttttaatcttcgatctgttaggtgttgatgtttacaagtgaattctagtgttttctcgttgagatctgtgaagtttgaacctagttttctcaataatcaacatatgaagcgatgtttgagtttcaataaacgctgctaatcttcgaaactaagttgtgatctgattcgtgtttacttcatgagcttatccaattcatttcggtttcattttactttttttttagtgaaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaggatcaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatccaacgttataacttcgtataatgtatgctatacgaagttattaactataacggtcctaaggtagcgacttaggctgagcccgggcaggcctacccataatacccataatagctgtttgccaatcgttcttcttggcgcgccagacatcctggaccaatatgctgaagattatgctacctacaccaggataggacttgaagcacttaaccttgaagattggttcgaagaaccagaacccgatccacctaaccctgtggaccgccagaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcctatataagggcattctcccctcagttgaagatcatcgaaagttggagcaataaactctctcttcaacaaatctatcttttatcttttatcggtaccaaaaaatggcgggatctaagaagagaagaattaaacaagatgacaagaagtatagtattggactcgatatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgacgctatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgatagcggcggcagctcgggcggcagctcgggctccgaaacgccggggaccagcgagagcgccacgcctgagagttccggcggaagcagcggcgggagttccaccctgaacatcgaggacgagtatcggcttcatgagaccagcaaagagccggacgtctccctgggaagcacctggctgagcgacttcccgcaagcctgggcggagaccggtggtatggggctcgcagtgcggcaagcgccgttgataatcccgctaaaggccacgagcacgcccgtgtctatcaagcagtacccgatgagtcaagaggcacgcctcggtatcaagccgcatatccagcgcctcctggaccagggcatcctcgtgccctgccagtctccctggaatacgcctctgctacccgtcaagaagcctggcaccaacgattacaggccggtgcaagacctgcgtgaggtcaacaagcgcgtggaggacatccacccaacggtgcccaacccgtacaatctcctatctggccttccgccctcgcaccagtggtacacggtcttggacctaaaggacgcattcttctgtctgaggctgcaccctacgtcccagccgctgttcgccttcgagtggcgcgacccggagatgggcatctctggccagctaacttggacgcgattgccccaggggtttaagaactcgcccacactcttcaacgaggcactccaccgtgacctggccgactttcgcatacagcaccccgaccttatcctgttgcagtacgtcgatgacctgctcctggcggccacgtccgagctggactgccagcaaggcacccgcgccctacttcaaaccctgggcaacctgggttaccgtgcgtccgccaagaaggcccaaatctgccaaaagcaagtcaagtacctcggctacctcttgaaggagggacagcgctggctgacggaggcgaggaaggagacggtgatgggtcagcccacacccaagaccccgaggcagctaagggagttcctggggaaggcgggcttctgccgtctattcatccctggcttcgcggagatggcggccccgctgtacccgctaacgaagccgggcacgctgttcaactggggccctgaccagcagaaggcgtaccaggagatcaagcaagcgttgcttactgccccagcactcggcctccccgacctcacaaagccgttcgagctattcgttgacgagaaacagggctacgcgaagggtgtgctgactcaaaagctagggccgtggcgacgcccagtagcctacctgagcaagaagctcgaccccgtggcggcgggctggccaccatgcctccggatggtcgcggctatcgcggtgctcacaaaggatgcggggaagctcacgatggggcagcccctggtgatcctggccccacacgcggtggaggcgttagtgaagcaacctcccgaccgatggctgagcaacgcccgcatgacccactaccaggcgctcctcctcgacaccgaccgcgtgcaattcggccctgtcgtggcactgaacccggccacgctgctcccactgcccgaggaggggctacagcacaactgcctcgatatactggcggaagcccacggcacccgccccgacttgacggaccagccgcttcccgacgcggaccatacgtggtacaccgacgggagttccttactccaagagggccagcgaaaggcgggcgctgcggtgaccactgagacggaagtaatctgggcaaaggcgctgcctgcgggcacgtctgcccagcgggcggagctgatcgccctgacccaggccctcaagatggccgagggcaagaagctgaacgtttacaccgacagtcggtatgccttcgcaactgcccacatccacggcgaaatctaccgtcggcgcggctggctgacgagcgagggcaaggagatcaagaacaaggacgagatcctcgccctgctaaaggcactcttcctgcccaagcgactgtccatcattcactgtccggggcaccagaagggccattccgccgaggcgcggggcaaccgcatggcggatcaggccgctcggaaggcggcgatcaccgagacgcccgatacgagcacgctcctgattgaaaactcgtcgccggggagcaagaaaaggcggatcaagcaagactagttaattaaagggctctctgtcatgatttcatactttcattattgagctctgtaattacaattatgaccatgagaacatctcttattgtgtggccttttaattgctgatgttagtactgaaccaaagcttatcgtgatgatgtaaaagcaataagtacttgtttgtagcttctttgtgtctccctttgggcttaatacatctgtttagtgttgtggctttggcatagacttctcttggtaataatgccttgcaatgcaaaatttcaattatcaaattctattatgttctcaccttatggtaacagcttaccctgtggaagatgagattcttgagttgagtcattgccaatttttggcattagcttttgaattagtgaattttgacaaaaattaccgtgacactgattttgttgaagctcttaagtgtagtttttacaaaatttcagtggctcgttgtgattatgtcaaactcacggcgaatgtagttcttacagaatttcagtggctcgggcccggccgtgacggccacgagcgaactcctgcaggggcatgtgactttttatttaaattattctcagaaatattaaaatataatcaaatatatttttttataagatactggaaataacatttttaggaatgtaacgaaagccccattacaaacacttgaactcagggatcgatccaacttaattattatctgctacttcaaattcaaatttataggccctacctttatgttttgctctgcactttcttaaatggaaacaagtaacacaatagagtaagatcatttacttgtaagcggaaactgttgcatcaactgcaatattttcgggttagttttataatataacaatgaagagaattgccgagaagatcatatcaagtcccatattccatttattggcctttacaagtcccacatcggtccaaatattagacaaagaaacatttatatatcgtctgacaaacctgtaagattgccgttaatttgagagtccagttttagagctagaaatagcaagttaaaataaggctagtccgttatcaacttgaaaaagtggcaccgagtcggtgcagctgaattaactctcaaatttttttttcgataaaaatgttttaaacgatatatattataaaaaaaaacgtttcaaaaataaatacaaaaatgtttttaaatatatataatttaactcattaaagaaaataaaaatgcaagtgcggtgacaagacaagctaaaagttgcaaaagaaatggcagggctataaggctcacctactcctggatttaccaaattttggttcgtccctatactcgaaaaataaaacaaaataaatttcagtatcttcgtttttgtatgctttgactgtgaggcgaggccaactttcttcttctgtctgagatgaattttgtttgcctcctgtgaaggatgtatcattcaaagtgaatgttttgcaactgccagtagtcccacatcgaccaaatattcttattacagtgtgtttatatagcacctggagaaggaatgggttgaatccttaacttatgccccagttttagagctagaaatagcaagttaaaataaggctagtccgttatcaacttgaaaaagtggcaccgagtcggtgctttttttgcggccgcacaacaaacgcgccggcgctctcttaaggtagc

SEQ ID NO: 145 - DNA, 67 bp, Nicotiana benthamiana
  source 1..67: mol_type = genomic DNA
agtaaaatgccccaaattggacttgtttctgccgttaatttgagagtccaaggtaattcagcttatc

SEQ ID NO: 146 - DNA, 50 bp, synthetic construct
  misc_feature 1..50: note = second row of Fig. 17
  source 1..50: mol_type = other DNA
ccaaattggacttgtttctgccgttaatttgagagttaattcagctgctc

SEQ ID NO: 147 - DNA, 24 bp, synthetic construct
  misc_feature 1..24: note = bottom row of Fig. 17
  source 1..24: mol_type = other DNA
aatttgagagttaattcagctgca

SEQ ID NO: 148 - DNA, 34 bp, Bacteriophage P1
  source 1..34: mol_type = genomic DNA
ataacttcgtataatgtatgctatacgaagttat

SEQ ID NO: 149 - DNA, 34 bp, synthetic construct
  misc_feature 1..34: note = lox75
  source 1..34: mol_type = other DNA
ataacttcgtataatgtatgctatacgcccggta

SEQ ID NO: 150 - DNA, 34 bp, synthetic construct
  misc_feature 1..34: note = lox76
  source 1..34: mol_type = other DNA
taccgggcgtataatgtatgctatacgaagttat

SEQ ID NO: 151 - DNA, 34 bp, synthetic construct
  misc_feature 1..34: note = lox66
  source 1..34: mol_type = other DNA
taccgttcgtataatgtatgctatacgaagttat

SEQ ID NO: 152 - DNA, 34 bp, synthetic construct
  misc_feature 1..34: note = lox71
  source 1..34: mol_type = other DNA
ataacttcgtataatgtatgctatacgaacggta

SEQ ID NO: 153 - DNA, 34 bp, synthetic construct
  misc_feature 1..34: note = lox78
  source 1..34: mol_type = other DNA
taccgggcgtataatgtatgctatacgcccggta

SEQ ID NO: 154 - DNA, 34 bp, synthetic construct
  misc_feature 1..34: note = lox72
  source 1..34: mol_type = other DNA
taccgttcgtataatgtatgctatacgaacggta

SEQ ID NO: 155 - DNA, 34 bp, Saccharomyces cerevisiae
  source 1..34: mol_type = genomic DNA
gaagttcctattctctagaaagtataggaacttc

SEQ ID NO: 156 - DNA, 31 bp, Zygosaccharomyces rouxii
  source 1..31: mol_type = genomic DNA
ttgatgaaagaatacgttattctttcatcaa

SEQ ID NO: 157 - DNA, 39 bp, Bacteriophage phi-C31
  source 1..39: mol_type = genomic DNA
ccccaactggggtaacctttgagttctctcagttggggg

SEQ ID NO: 158 - DNA, 34 bp, Bacteriophage phi-C31
  source 1..34: mol_type = genomic DNA
gtgccagggcgtgcccttgggctccccgggcgcg

SEQ ID NO: 159 - DNA, 48 bp, unidentified
  source 1..48: mol_type = genomic DNA; note = bacteriophage Bxb1
ggtttgtctggtcaaccaccgcggtctcagtggtgtacggtacaaacc

SEQ ID NO: 160 - DNA, 42 bp, unidentified
  source 1..42: mol_type = genomic DNA; note = bacteriophage Bxb1
ccggcttgtcgacgacggcggtctccgtcgtcaggatcatcc

SEQ ID NO: 161 - DNA, 10 bp, synthetic construct
  misc_feature 1..10: note = reverse transcriptase target sequence
  source 1..10: mol_type = other DNA
tgagtctggc

(19) United States
(12) Patent Application Publication
(10) Pub. No.: US 2025/0368972 A1
(43) Pub. Date: Dec. 4, 2025

(54) COMPOSITIONS AND METHODS FOR RNA-TEMPLATED EDITING IN PLANTS

(71) Applicants: Pairwise Plants Services, Inc., Durham, NC (US); Monsanto Technology LLC, St. Louis, MO (US)

(72) Inventors: Aaron Hummel, Hillsborough, NC (US); Joseph Matthew Watts, Cary, NC (US); Shai Joshua Lawit, Durham, NC (US); Nathaniel Graham, Durham, NC (US); Larry Gilbertson, St. Louis, MO (US); Ervin Nagy, St. Louis, MO (US); Linda Rymarquis, St. Louis, MO (US)

(21) Appl. No.: 19/205,140

(22) Filed: May 12, 2025

Related U.S. Application Data
(63) Continuation of application No. 17/078,919, filed on Oct. 23, 2020, now Pat. No. 12,331,330.
(60) Provisional application No. 62/925,097, filed on Oct. 23, 2019.

(51) Int. Cl. C12N 9/22

(52) U.S. Cl. CPC C12N 9/22; C07K 2319/80; C12N 2310/20

(57) ABSTRACT

This invention relates to recombinant nucleic constructs comprising a DNA binding domain, an endonuclease and a reverse transcriptase and methods of use thereof for modifying nucleic acids in plants.

### STATEMENT REGARDING ELECTRONIC FILING OF A SEQUENCE LISTING

A Sequence Listing in XML format, entitled 1499-11CT_ST26.xml, 329,626 bytes in size, generated on Aug. 20, 2025, and filed herewith, is hereby incorporated by reference in its entirety for its disclosures.

### FIELD OF THE INVENTION

This invention relates to recombinant nucleic acid constructs encoding a DNA binding polypeptide, an endonuclease and/or a reverse transcriptase and to methods of modifying a nucleic acid in a plant.

### BACKGROUND OF THE INVENTION

Base editing has been shown to be an efficient way to change cytosine and adenine residues to thymine and guanine, respectively. These tools, while powerful, do have some limitations such as bystander bases, small base editing windows that give limited accessibility to trait-relevant targets unless enzymes with high PAM density are available to compensate, limited ability to convert cytosines and adenines to residues other than thymine and guanine, respectively, and no ability to edit thymine or guanine residues. Thus, the current tools available for base editing are limited, particularly in plants. Therefore, to make nucleic acid editing more useful across a greater number of organisms, including plants, new editing tools are needed.

### SUMMARY OF THE INVENTION

A first aspect of the present invention is directed to a method of modifying a target nucleic acid in a plant cell, the method comprising: contacting the target nucleic acid with (a) a DNA binding domain (e.g., a first DNA binding domain); (b) a DNA endonuclease (e.g., a first DNA endonuclease); and (c) a reverse transcriptase (e.g., a first reverse transcriptase), thereby modifying the target nucleic acid in the plant cell.

Another aspect of the present invention is directed to an expression cassette codon optimized for expression in a plant, comprising 5′ to 3′ (a) polynucleotide encoding a plant specific promoter sequence (e.g., ZmUbi1, MtUb2, RNA polymerase II (Pol II)), (b) a plant codon-optimized polynucleotide encoding a CRISPR-Cas nuclease (e.g. nCas9, dCas9, Cpf1 (Cas12a), dCas12a and the like); (c) a linker sequence; and (d) a plant codon-optimized polynucleotide encoding a reverse transcriptase.

A further aspect of the present invention is directed to an expression cassette codon optimized for expression in a plant, comprising: (a) a polynucleotide encoding a plant specific promoter sequence (e.g., ZmUbi1, MtUb2), and (b) an extended guide nucleic acid, wherein the extended guide nucleic acid comprises an extended portion comprising at its 3′ end a primer binding site and an edit to be incorporated into the target nucleic acid (e.g., reverse transcriptase template), optionally wherein the extended guide nucleic acid is comprised in an expression cassette, optionally wherein the extended guide nucleic acid is operably linked to a Pol II promoter.

An additional aspect of the present invention is directed to a method of modifying a target nucleic acid in a plant cell, comprising contacting the target nucleic acid with a DNA binding domain and a DNA endonuclease domain targeted to a first site on the target nucleic acid and the same or a different DNA binding domain and DNA endonuclease domain targeted to a second site on the target nucleic acid, wherein the first site and the second site are proximal to one another on the same (nontarget) strand, thereby nicking the target nucleic acid at the first and second site; a reverse transcriptase; and a nucleic acid encoded repair template encoding a modification to be incorporated into the target nucleic acid, thereby modifying the target nucleic acid in the plant.

Another aspect of the present invention is directed to a method of modifying a target nucleic acid in a plant cell, the method comprising: contacting the target nucleic acid with (a) a CRISPR-Cas nuclease comprising a first DNA binding domain and a first DNA endonuclease (a nickase); (b) a reverse transcriptase; (c) a CRISPR RNA (crRNA) comprising a spacer having substantial homology to a first site on the target nucleic acid; (d) a trans-activating crRNA (tracrRNA) that interacts (recruits/binds) with the crRNA and the CRISPR-Cas nuclease; and (e) a nucleic acid encoded repair template (e.g., an RNA encoded repair template) comprising a primer binding site and an template encoding the modification to be incorporated into the target nucleic acid, wherein the tracrRNA comprises a sequence at the 5′ or 3′ end that is complementary to a sequence at the 5′end or 3′ end of the reverse transcriptase template, thereby modifying the target nucleic acid.

A further aspect of the present invention is directed to a method of modifying a target nucleic acid in a plant cell, the method comprising: contacting the target nucleic acid with (a) a CRISPR-Cas nuclease comprising a first DNA binding domain and a first DNA endonuclease (a nickase); (b) a reverse transcriptase; (c) a CRISPR RNA (crRNA) comprising a spacer having substantial homology to a first site on the target nucleic acid; (d) a trans-activating crRNA (tracrRNA) that interacts (recruits/binds) with the crRNA and the CRISPR-Cas nuclease; and (e) a nucleic acid encoded repair template (e.g., an RNA encoded repair template) comprising a primer binding site and an template encoding the modification to be incorporated into the target nucleic acid, thereby modifying the target nucleic acid.

Another aspect of the present invention is directed to a method of modifying a target nucleic acid in a plant cell, the method comprising: contacting the target nucleic acid with (a) a CRISPR-Cas nuclease comprising a first DNA binding domain and a first DNA endonuclease (a nickase); (b) a reverse transcriptase; (c) a CRISPR RNA (crRNA) guide that interacts (recruits/binds) with the CRISPR-Cas nuclease and comprises a spacer having substantial homology to a first site on the target nucleic acid; and (e) a nucleic acid encoded repair template (e.g., an RNA encoded repair template) comprising a primer binding site and an RNA template (that encodes the modification to be incorporated into the target nucleic acid), wherein the crRNA comprises a sequence at its 5′ end or 3′ end that is complementary to the primer binding site, thereby modifying the target nucleic acid.

A further aspect of the present invention is directed to a method of modifying a target nucleic acid in a plant cell, the method comprising: contacting the target nucleic acid with (a) a CRISPR-Cas nuclease comprising a first DNA binding domain and a first DNA endonuclease (e.g., a nickase); (b) a reverse transcriptase; (c) an extended guide nucleic acid comprising a sequence that interacts that interacts (recruits/binds) with the CRISPR-Cas nuclease and a spacer having substantial homology to a first site on the target nucleic acid (e.g., CRISPR RNA (crRNA) (a first crRNA) and/or tracrRNA+crRNA (sgRNA)) and a nucleic acid encoded repair template (e.g., an RNA encoded repair template) comprising a primer binding site and an RNA template (that encodes the modification to be incorporated into the target nucleic acid), thereby modifying the target nucleic acid.

An additional aspect of the present invention is directed to a method of modifying a target nucleic acid in a plant cell, the method comprising: contacting the target nucleic acid with (a) a first CRISPR-Cas nuclease (a nickase) comprising a first DNA binding domain and a first DNA endonuclease; (b) an extended guide nucleic acid comprising a CRISPR RNA (crRNA) comprising a spacer having substantial homology to a first site on the target nucleic acid, a trans-activating crRNA (tracrRNA) that recruits the first CRISPR-Cas nuclease and an RNA template comprising the modification to be incorporated into the target nucleic acid, wherein the first CRISPR-Cas nuclease nicks the target nucleic acid at a first site (on the non-target strand); (c) a second CRISPR Cas-nuclease (a nickase) comprising a first DNA binding domain and a first DNA endonuclease (a nickase); (d) a guide nucleic acid comprising a CRISPR RNA (crRNA) comprising a spacer having substantial homology to a second site on the target nucleic acid that is proximal to (and on the same strand as) the first site on the target nucleic acid, a trans-activating crRNA (tracrRNA) that recruits the second CRISPR-Cas nuclease, thereby nicking the DNA at the second site (on the non-target strand); and (e) a reverse transcriptase fused or recruited to the first CRISPR Cas-nuclease and/or the second CRISPR Cas-nuclease, thereby modifying the target nucleic acid.

A further aspect of the present invention is directed to a method of releasing a portion of a double stranded nucleic acid, comprising: (a) targeting a first DNA endonuclease to a first site of the nucleic acid; (b) making a nick at in a first strand of the nucleic acid at the first site; (c) targeting the first DNA endonuclease or a second DNA endonuclease to a second site on the first strand; and (d) making a nick in the first strand at the second site, wherein the portion of the first strand of the nucleic acid between the first site and second site can be released from the nucleic acid.

The invention further provides expression cassettes and/or vectors comprising a nucleic acid construct of the present invention, and cells comprising a polypeptide, fusion protein and/or nucleic acid construct of the present invention. Additionally, the invention provides kits comprising a nucleic acid construct of the present invention and expression cassettes, vectors and/or cells comprising the same.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 provides a schematic showing the generation of DNA sequences from reverse transcription off the sgRNA and subsequent integration into the nick site. The extended sgRNA is shown in light gray and is bound to the non-target strand nickase Cas9 (nCas9, upper left). The 3′ end of the sgRNA is complimentary to the DNA at the nick site (black pairing lines, upper left). The RT then polymerizes DNA from the 3′ end of the DNA nick generating a DNA sequence with non-complimentary nucleotides (pairing lines indicated by bracket, upper right) followed by complimentary nucleotides (black pairing lines, upper right). Upon dissociation, the resultant DNA has an extended ssDNA with a 3′ overhang which is largely the same sequence as the original DNA (black pairing lines, lower right) but with some non-native nucleotides (pairing lines indicated by bracket, lower right). This flap is in equilibrium with a structure having a 5′ overhang (lower left) where there are mismatched nucleotides incorporated into the DNA.

FIG. 2 provides a schematic of reducing mismatch repair. In order to drive the equilibrium more in favor of forming the final product with the modified nucleotides (indicated by bracket), a target strand (TS) nickase is targeted to regions outside of the RT-editing bubble (lightning bolts). The nCas9:sgRNA molecules may be on either side or both sides of the editing bubble. Nicking the target strand (dashed line) indicates to the cell that the newly incorporated nucleotides are the correct nucleotides during mismatch repair and replication, thus favoring a final product with the new nucleotides.

FIG. 3 shows alternative methods of modifying nucleic acids using the compositions of the present invention, wherein in two nicks are introduced in the PAM-containing strand and the sequence introduced by the RT displaces the double-nicked WT sequence and thereby, is more efficiently incorporated into the genome.

FIG. 4 is an illustration of an exemplary effector sequence including a nickase (Cas9 (H840A)) (white) followed by a linker (black) which is followed by eight repeats of the GCN4 epitope motif.

FIG. 5 is an illustration of an exemplary sequence including a scFv fragment (grey) that is fused to the reverse transcriptase MuL V-5M followed by the guanine nucleotide-binding protein subunit beta sequence.

FIG. 6 is an illustration of an exemplary pegRNA structure where the promoter (Hs.U6) is promoting the transcription of a spacer, followed by the sgRNA scaffold, reverse transcriptase template (RT Template), and primer binding site (PBS).

FIG. 7 is a graph showing the results of editing at the FANCF1 locus using the recruitment (Suntag) strategy or published PE2 strategy.

FIG. 8 is a graph showing the results of editing at the DMNTI locus using the recruitment (Suntag) strategy or published PE2 strategy.

FIG. 9 is a graph showing the results of editing at the RUNX1 locus using the recruitment (Suntag) strategy or published PE2 strategy.

FIG. 10 is a graph showing the results of editing at the RNF2 locus using the recruitment (Suntag) strategy or published PE2 strategy.

FIG. 11 is an illustration of the strategy to recruit the reverse transcriptase to an upstream template through a guide on the opposite strand. The reverse transcriptase is recruited to the pegRNA through a secondary guide containing a MS2 stem loop.

FIG. 12 is a diagram of two target sites for testing of reverse transcriptase recruitment through MS2 loops. Spacer binding sites are shown with arrows and designed changes shown in boxes. WT sequence for site 02 (SEQ ID NO: 77); Edit sequence for site 02 (SEQ ID NO: 78); WT sequence for site 03 (SEQ ID NO: 79); and Edit sequence for site 03 (SEQ ID NO: 80).

FIG. 13 is an illustration of the strategy to recruit the reverse transcriptase to an upstream template through a guide on the same strand. The reverse transcriptase is recruited to the pegRNA through a secondary guide containing a MS2 stem loop.

FIG. 14 is a diagram showing evidence of recruitment reverse transcriptase editing at the 02 site. Edits are shown in light gray and indicated by brackets. Top line: SEQ ID NO: 81; Bottom line: SEQ ID NO: 82.

FIG. 15 is a diagram showing evidence of recruitment reverse transcriptase editing at the 03 site. Edits are shown in light gray and indicated by brackets. Top line: SEQ ID NO: 83; Middle line: SEQ ID NO: 84; Bottom line: SEQ ID NO: 85.

FIG. 16 is an illustration of a structure of pegRNA for the experiment in tobacco.

FIG. 17 is a diagram showing evidence of prime editing in plants. The top row is the targeted gene sequence (SEQ ID NO: 130) with the spacer and primer binding site annotated. The second row (SEQ ID NO: 131) is the amplicon sequencing result aligned to the reference showing the targeted deletion and insertion. The bottom row (SEQ ID NO: 132) shows the prime binding site, reverse transcriptase template, and the first three bases of the scaffold, demonstrating the source of the deletion and insertion.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the reference organism. A “homologous” nucleic acid sequence is a nucleotide sequence naturally associated with a host cell into which it is introduced.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “recombinant nucleic acid,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., “substantially complementary” such as about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

A “portion” or “fragment” of a nucleotide sequence or polypeptide will be understood to mean a nucleotide sequence or polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide sequence or polypeptide, respectively, and comprising, consisting essentially of and/or consisting of contiguous residues identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide sequence or polypeptide. Such a nucleic acid fragment or portion according to the invention may be, where appropriate, included in a larger polynucleotide of which it is a constituent. As an example, a repeat sequence of guide nucleic acid of this invention may comprise a portion of a wild type CRISPR-Cas repeat sequence (e.g., a wild Type CRISR-Cas repeat; e.g., a repeat from the CRISPR Cas system of a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like).

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCGR Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1× SSC at 45° C. for 15 minutes. An example of a low stringency wash for a duplex of, e.g., more than 100 nucleotides, is 4-6× SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention can be codon optimized for expression. In some embodiments, a polynucleotide, nucleic acid construct, expression cassette, and/or vector of the invention (e.g., comprising/encoding a DNA binding domain, a DNA endonuclease, a reverse transcriptase, a flap endonuclease, and/or the like) are codon optimized for expression in an organism (e.g., an animal, a plant (e.g., in a particular plant species), a fungus, an archaeon, or a bacterium). In some embodiments, the codon optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acid constructs, polynucleotides, expression cassettes, and/or vectors but which have not been codon optimized.

In any of the embodiments described herein, a polynucleotide or nucleic acid construct of the invention may be operatively associated with a variety of promoters and/or other regulatory elements for expression in an organism or cell thereof (e.g., a plant and/or a cell of a plant). Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked” or “fused” in reference to polypeptides, refers to the attachment of one polypeptide to another. A polypeptide may be linked (e.g., fused) to another polypeptide (at the N-terminus or the C-terminus) directly (e.g., via a peptide bond) or through a linker (e.g., a peptide linker).

The term “linker” in reference to polypeptides is art-recognized and refers to a chemical group, or a molecule linking two molecules or moieties, e.g., two domains of a fusion protein, such as, for example, a fusion protein comprising a DNA binding polypeptide (e.g., a DNA binding domain) and a peptide tag (e.g., a peptide repeat unit), a fusion protein comprising a a reverse transcriptase and an affinity polypeptide that binds to the peptide tag, a fusion protein comprising a DNA endonuclease polypeptide (e.g., a DNA binding domain) and peptide tag, and/or a fusion protein comprising a reverse transcriptase and an affinity polypeptide that binds to the peptide tag. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g. extension of the hairpin structure in guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, a promoter region may comprise at least one intron (e.g., SEQ ID NOs: 1 or 2).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79:87-94), maize (Christensen et al., 1989. Plant Molec. Biol. 12:619-632), and arabidopsis (Norris et al. 1993. Plant Molec. Biol. 21:895-906). The maize ubiquitin promoter ((bi) has been developed in transgenic monocot systems and its sequence and vectors constructed for monocot transformation are disclosed in European patent publication EP0342926. The ubiquitin promoter is suitable for the expression of the nucleotide sequences of the invention in transgenic plants, especially monocotyledons. Further, the promoter expression cassettes described by McElroy et al. (Mol. Gen. Genet. 231:150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP U.S. Pat. No. 255,378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, incorporated herein by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); European patent EP0452269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9 (5):297-306 (2015)), ZmSTK2_USP from maize (Wang et al. Genome 60 (6):485-495 (2017)), LAT52 and LAT59 from tomato (Twell et al. Development 109 (3):705-713 (1990)), Zm13 (U.S. Pat. No. 10,421,972), PLA2-8 promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37 (8):1108-1115), corn light harvesting complex promoter (Bansal et al. (1992) Proc. Natl. Acad. Sci. USA 89:3654-3658), corn heat shock protein promoter (O'Dell et al. (1985) EMBO) J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a nucleic acid construct of the invention (e.g., a DNA binding polypeptide or domain (e.g. a CRISPR-Cas nuclease, a transcription activator-like effector (TALE) protein domain or polypeptide, and/or a zinc finger protein domain or polypeptide), an endonuclease polypeptide or domain (e.g., a CRISPR-Cas nuclease, and/or a Fok1 endonuclease), a reverse transcriptase polypeptide or domain, and/or a flap endonuclease polypeptide or domain (e.g., FEN)), wherein the nucleic acid construct is operably associated with at one or more control sequences (e.g., a promoter, terminator and the like). Thus, some embodiments of the invention provide expression cassettes designed to express, for example, a nucleic acid construct of the invention (e.g., a nucleic acid construct of the invention encoding a DNA binding polypeptide or domain, an endonuclease polypeptide or domain, a reverse transcriptase polypeptide or domain, a flap endonuclease polypeptide or domain and/or nucleic acid modifying polypeptide or domain. When an expression cassette comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter or they may be different promoters. Thus, a polynucleotide encoding a DNA binding polypeptide or domain, a polynucleotide encoding an endonuclease polypeptide or domain, a polynucleotide encoding a reverse transcriptase polypeptide or domain, a polynucleotide encoding a flap endonuclease polypeptide or domain and/or a polynucleoptide encoding a nucleic acid modifying polypeptide or domain comprised in an expression cassette may each be operably linked to a separate promoter or they may be operably linked to two or more promoters in any combination.

In some embodiments, an expression cassette and the polynucleotides comprised therein in may be optimized for expression in an organism (e.g., an animal, a plant, a bacterium and the like).

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to a gene encoding a DNA binding polypeptide, a gene encoding an endonuclease polypeptide, a gene encoding a reverse transcriptase, a gene encoding a flap endonuclease, and/or a gene encoding a nucleic acid modifying polypeptide nuclease, may be native to a host cell, or may be native to another source (e.g., foreign or heterologous to the promoter, to a gene encoding the DNA binding polypeptide, to the gene encoding an endonuclease polypeptide, to the gene encoding a reverse transcriptase, to the gene encoding a flap endonuclease, to the gene encoding a nucleic acid modifying polypeptide nuclease, to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

The expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid construct or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a nucleic acid binding domain (e.g., a DNA binding domain such as a sequence-specific DNA binding protein (e.g., polynucleotide-guided endonuclease, a CRISPR-Cas effector protein (e.g., CRISPR-Cas endonuclease), a zinc finger nuclease, a transcription activator-like effector nuclease (TALEN) and/or an Argonaute protein), and a reverse transcriptase or a nucleic acid construct encoding the same, under conditions whereby the nucleic acid binding domain (e.g., CRISPR-Cas nuclease) and the reverse transcriptase are expressed and the nucleic acid binding domain binds to the target nucleic acid, and the reverse transcriptase is either fused to the nucleic acid binding domain or is recruited to the nucleic acid binding domain (e.g., via a peptide tag (e.g., peptide repeat unit) fused to the nucleic acid binding domain and an affinity tag fused to the reverse transcriptase) (and thus, the reverse transcriptase is positioned in the vicinity of the target nucleic acid), thereby modifying the target nucleic acid. In some embodiments, the reverse transcriptase and the nucleic acid binding domain (e.g., CRISPR-Cas endonuclease) localize at the target nucleic acid, optionally through covalent and/or non-covalent interactions.

As used herein, “modifying” or “modification” in reference to a target nucleic acid includes editing (e.g., mutating), covalent modification, exchanging/substituting nucleic acids/nucleotide bases, deleting, cleaving, nicking, and/or transcriptional control of a target nucleic acid. In some embodiments, a modification may include an indel of any size and/or a single base change (SNP) of any type.

“Recruit,” “recruiting” or “recruitment” as used herein refer to attracting one or more polypeptide(s) or polynucleotide(s) to another polypeptide or polynucleotide (e.g., to a particular location in a genome) using protein-protein interactions, RNA-protein interactions, and/or chemical interactions. Protein-protein interactions can include, but are not limited to, peptide tags (epitopes, multimerized epitopes) and corresponding affinity polypeptides, RNA recruiting motifs and corresponding affinity polypeptides, and/or chemical interactions. Example chemical interactions that may be useful with polypeptides and polynucleotides for the purpose of recruitment can include, but are not limited to, rapamycin-inducible dimerization of FRB-FKBP; Biotin-streptavidin interaction; SNAP tag (Hussain et al. Curr Pharm Des. 19 (30):5437-42 (2013)); Halo tag (Los et al. ACS Chem Biol. 3 (6):373-82 (2008)); CLIP tag (Gautier et al. Chemistry & Biology 15:128-136 (2008)); DmrA-DmrC heterodimer induced by a compound (Tak et al. Nat Methods 14 (12):1163-1166 (2017)); Bifunctional ligand approaches (fuse two protein-binding chemicals together) (Voß et al. Curr Opin Chemical Biology 28:194-201 (2015)) (e.g. dihyrofolate reductase (DHFR) (Kopyteck et al. Cell Cehm Biol 7(5):313-321 (2000)).

“Introducing,” “introduce,” “introduced” (and grammatical variations thereof) in the context of a polynucleotide of interest means presenting a nucleotide sequence of interest (e.g., polynucleotide, a nucleic acid construct, and/or a guide nucleic acid) to a host organism or cell of said organism (e.g., host cell; e.g., a plant cell) in such a manner that the nucleotide sequence gains access to the interior of a cell. Thus, for example, a nucleic acid construct of the invention encoding a DNA binding domain, a DNA endonuclease, and/or a reverse transcriptase may be introduced into a cell of an organism, thereby transforming the cell with the DNA binding domain, the DNA endonuclease, and/or the reverse transcriptase.

The terms “transformation” or transfection” may be used interchangeably and as used herein refer to the introduction of a heterologous nucleic acid into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes encoding a DNA binding polypeptide or domain, an endonuclease polypeptide or domain, a reverse transcriptase polypeptide or domain, a flap endonuclease polypeptide or domain and/or nucleic acid modifying polypeptide or domain) may be transiently introduced into a cell with a guide nucleic acid and as such, no DNA maintained in the cell.

A nucleic acid construct of the invention can be introduced into a cell by any method known to those of skill in the art. In some embodiments of the invention, transformation of a cell comprises nuclear transformation. In other embodiments, transformation of a cell comprises plastid transformation (e.g., chloroplast transformation). In still further embodiments, the recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)).

A nucleotide sequence therefore can be introduced into a host organism or its cell in any number of ways that are well known in the art. The methods of the invention do not depend on a particular method for introducing one or more nucleotide sequences into the organism, only that they gain access to the interior of at least one cell of the organism. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, and/or in separate transformation events, or, alternatively, where relevant, a nucleotide sequence can be incorporated into a plant, for example, as part of a breeding protocol.

Base editing has been shown to be an efficient way to change cytosine and adenine residues to thymine and guanine, respectively. These tools, while powerful, do have some limitations such as bystander bases, small base editing windows, and limited PAMs.

To perform precise templated editing in cells there are several essential steps, each of which has rate limitations that together can severely hamper the ability to effectively perform editing due to low efficiencies. For example, one step requires inducing the cell to initiate a repair event at the target site. This is typically performed by causing a double-strand break (DSB) or nick by an exogenously provided, sequence-specific nuclease or nickase. Another step requires local availability of a homologous template to be used for the repair. This step requires the template to be in the proximity of the DSB at exactly the right time when the DSB is competent to commit to a templated editing pathway. In particular, this step is widely regarded to be the rate limiting step with current editing technologies. A further step is the efficient incorporation of sequence from the template into the broken or nicked target. Prior to the present invention, this step was typically provided by the cell's endogenous DNA repair enzymes. The efficiency of this step is probably low and is very difficult to manipulate. The present invention bypasses many of the major obstacles to the efficiency of the process of templated editing by co-localizing, in a coordinate fashion, the functionalities required to carry out the steps described above.

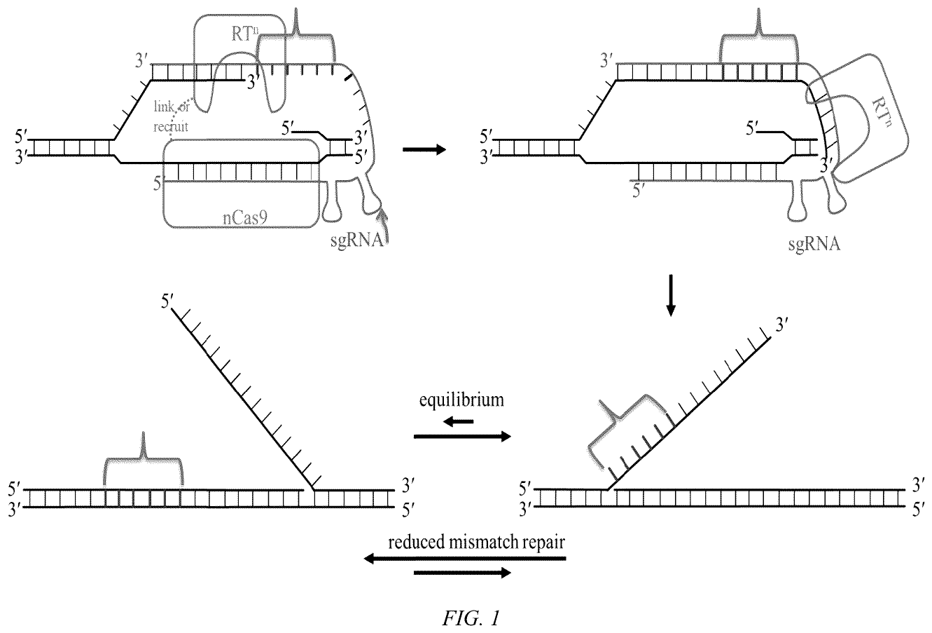

FIG. 1 shows the generation of DNA sequences from reverse transcription off the sgRNA and subsequent integration into the nick site using methods and constructs of the present invention. An extended sgRNA is shown in light gray and is bound to the non-target strand nickase Cas9 (nCas9, upper left) (e.g., a binding domain and a DNE endonuclease domain (e.g. H840A)). As described in more detail herein, the nCas9 may be either covalently linked via, for example, a peptide to a reverse transcriptase (RT) or the RT may be recruited to the nCas9 (e.g., via the use of a peptide repeat unit motif/affinity polypeptide that binds to the peptide repeat unit as described herein), in which case multiple reverse transcriptase proteins (RTⁿ) may be recruited. The 3′ end of the sgRNA is complimentary to the DNA at the nick site (black pairing lines, upper left). The RT then polymerizes DNA from the 3′ end of the DNA nick generating a DNA sequence with non-complimentary nucleotides (pairing lines indicated by bracket, upper right) followed by complimentary nucleotides (black pairing lines, upper right). Upon dissociation, the resultant DNA has an extended ssDNA with a 3′ overhang which is largely the same sequence as the original DNA (black pairing lines, lower right) but with some non-native nucleotides (pairing lines indicated by bracket, lower right). This flap is in equilibrium with a structure having a 5′ overhang (lower left) where there are mismatched nucleotides incorporated into the DNA. This equilibrium lies more to the favorable perfect pairing on the right, but can be driven may be reduced in a variety of ways including, for example, nicking the target strand. The structure on the left is preferentially cleaved by cellular flap endonucleases involved in DNA lagging strand synthesis, which are highly conserved between mammalian and plant cells (the amino acid sequence of Homo sapiens FEN1 is over 50% identical to both Zea mays and Glycine max FEN1). Thus, a flap endonuclease may be introduced to drive the equilibrium in the direction of the 3′ flap comprising the non-native/mismatched nucleotides.

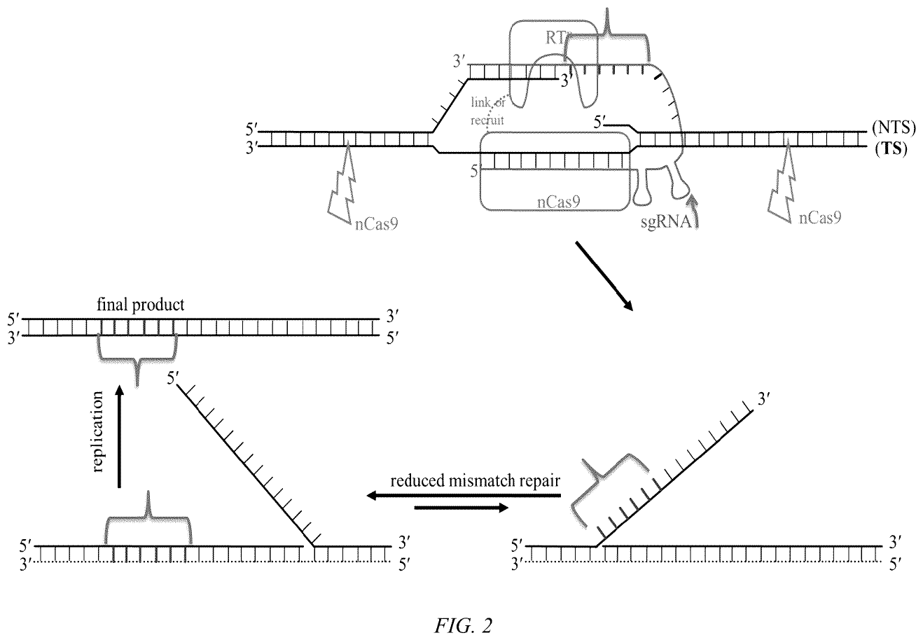

Further in the process of the present invention, and as exemplified in FIG. 2, to reduce mismatch repair and to drive the equilibrium more in favor of forming the final product with the modified nucleotides (indicated by bracket), a target strand (TS) nickase, for example, Cas9-D10A, is targeted to regions outside of the RT-editing bubble (lightning bolts). The nCas9:sgRNA molecules may be on either side or both sides of the editing bubble. Nicking the target strand (dashed line) indicates to the cell that the newly incorporated nucleotides are the correct nucleotides during mismatch repair and replication, thus favoring a final product with the new nucleotides.

Variants of the reverse transcriptase (RT) enzyme can have significant effects on the temperature-sensitivity and processivity of the editing system. Natural and rationally- and irrationally-engineered (i.e., directed evolution) variants of the RT may be useful in optimizing activity in plant-preferred temperatures and for optimizing processivity profiles.

Protein domain fusions to the RT enzyme can have significant effects on the temperature-sensitivity and processivity of the editing system. The RT enzyme can be improved for temperature-sensitivity, processivity, and template affinity through fusions to ssRNA binding domains (RBDs). These RBDs may have sequence specificity, non-specificity or sequence preferences. A range of affinity distributions may be beneficial to editing in different cellular and in vitro environments. RBDs can be modified in both specificity and binding free energy through increasing or decreasing the size of the RBD in order to recognize more or fewer nucleotides. Multiple RBDs result in proteins with affinity distributions that are a combination of the individual RBDs. Adding one or more RBD to the RT enzyme can result in increased affinity, increased or decreased sequence specificity, and/or promote cooperativity.

After reverse transcriptase incorporates the edit into the genome, a sequence redundancy exists between the newly synthesized edited sequence and the original WT sequence it is intended to replace. This leads to either a 5′ or 3′ flap at the target site, which has to be repaired by the cell. The two states exist in an equilibrium. Binding energy favors the 3′ flap because more base pairs are available when the WT sequence is paired with its complement than when the edited strand is paired with its complement. This is unfavorable for efficient editing because processing (removal) of the 3′ flap would remove the edited residues and revert the target back to WT sequence. However, cellular flap endonucleases such as FEN1 can efficiently process 5′ flaps. Thus, instead of relying on the function of 5′-flap endonucleases native to the cell, in some embodiments of this invention the concentration of flap endonucleases at the target may be increased to further favor the desirable equilibrium outcome (removal of the WT sequence in the 5′ flap so that the edited sequence becomes stably incorporated at the target site). This may be achieved by overexpression of a 5′ flap endonuclease as a free protein in the cell. Alternatively, FEN may be actively recruited to the target site by association with the CRISPR complex, either by direct protein fusion or by non-covalent recruitment such as with a peptide tag (e.g., a peptide repeat unit) and affinity polypeptide pair (e.g., a SunTag antibody/epitope pair).

Thus, in some embodiments, a method of modifying a target nucleic acid in a plant cell is provided, the method comprising: contacting the target nucleic acid with (a) a DNA binding domain (e.g., a first DNA binding domain); (b) a DNA endonuclease (e.g., a first DNA endonuclease); and (c) a reverse transcriptase (e.g., a first reverse transcriptase), thereby modifying the target nucleic acid. In some embodiments, the (a) DNA binding domain; (b) the DNA endonuclease; and (c) the reverse transcriptase are comprised in a complex. In some embodiments, the DNA binding protein is a DNA binding fusion protein comprising a DNA binding protein domain fused (linked) to a peptide tag (e.g., peptide repeat unit, an epitope or a multimerized epitope) and/or the DNA endonuclease is a DNA endonuclease fusion protein comprising a DNA endonuclease domain fused (linked) to a peptide tag (e.g., a peptide repeat unit, an epitope or a multimerized epitope) and the reverse transcriptase is a reverse transcriptase fusion protein comprising a reverse transcriptase domain fused (e.g., linked) to an affinity polypeptide that binds to the peptide tag, optionally wherein the target nucleic acid is contacted with two or more reverse transcriptase fusion proteins.

In some embodiments, the DNA binding domain may be a CRISPR-Cas nuclease domain, a transcription activator-like effector (TALE) protein domain, and/or a zinc finger protein domain. In some embodiments, the DNA endonuclease may be a CRISPR-Cas nuclease, and/or a Fok1 endonuclease. In some embodiments, a DNA binding domain (a) and/or a DNA endonuclease (b) may be comprised in a CRISPR-Cas nuclease. In some embodiments, the CRISPR-Cas nuclease is a Cas9 nickase (nCas9). In some embodiments, the DNA binding domain may be a CRISPR-Cas nuclease comprising a mutation in one or more nuclease active sites (e.g., in the RuvC domain, in the HNH domain) (e.g., deactivated or deadCas (dCas)), optionally a dCas9 or dCas12a. In some embodiments, the DNA endonuclease is a Fok1 endonuclease.

In some embodiments, a method of the invention may further comprise contacting the target nucleic acid with an extended guide nucleic acid (e.g., a pegRNA), wherein the extended guide nucleic acid comprises an extended portion comprising a primer binding site and a reverse transcriptase template, wherein the reverse transcriptase template comprises the edit to be incorporated into the target nucleic acid, optionally wherein the extended guide nucleic acid is comprised in an expression cassette, optionally wherein the extended guide nucleic acid is operably linked to a Pol II promoter.

In some embodiments, an extended guide RNA may comprise, 5′-3′, a spacer sequence, a repeat sequence, and an extended portion, the extended portion comprising, 5′ to 3′, a reverse transcriptase template and a primer binding site. In some embodiments, an extended guide RNA may comprise, 5′-3′, a spacer sequence, a repeat sequence and an extended portion, the extended portion comprising, 5′ to 3′, a primer binding site and a reverse transcriptase template. In some embodiments, an extended guide RNA may comprise, 5′-3′, an extended portion, a spacer sequence, and a repeat sequence, wherein the extended portion comprises, 5′ to 3′, a reverse transcriptase template and a primer binding site. In some embodiments, an extended guide RNA may comprise, 5′-3′, an extended portion, a spacer sequence, and a repeat sequence, wherein the extended portion comprises, 5′ to 3′, a primer binding site and a reverse transcriptase template.

In some embodiments, an extended guide nucleic acid may be linked to an RNA recruiting motif, and the reverse transcriptase may be a reverse transcriptase fusion protein comprising a reverse transcriptase domain fused (linked) to an affinity polypeptide that binds to the RNA recruiting motif, optionally wherein the target nucleic acid is contacted with two or more reverse transcriptase fusion proteins. In some embodiments, a reverse transcriptase may be recruited through RNA recruitment, which may direct the reverse transcriptase to the exact template location on the extended guide nucleic acid. In some embodiment, the extended guide nucleic acid comprises a peptide tag (e.g., a protein-recruitment scaffold such as, but not limited to, a MS2 phage operator stem-loop, a PP7 phage operator stem-loop or a SfMu phage Com stem-loop) that can be used to recruit the reverse transcriptase as the reverse transcriptase comprises an affinity polypeptide that corresponds to the peptide tag (e.g., a protein recruitment domain such as, but not limited to, a MS2 Coat Protein (MCP) polypeptide, a PP7 Coat Protein (PCP) polypeptide, or a Com RNA binding protein polypeptide).

According to some embodiments, an extended guide nucleic acid (e.g., a pegRNA) may have a structure and/or be designed as described in Anzalone et al., Nature, 2019 December; 576 (7785): 149-157. In some embodiments, an extended guide nucleic acid comprises a primer binding site (PBS) optionally having a sequence of 1, 2, 3, 4, or 5 to 6, 7, 8, 9, 10, 11, 12, 13, 14, or 15 nucleotides and a reverse transcriptase template (RT template) sequence optionally having a sequence of 65 nucleotides or more. In some embodiments, the PBS of the extended guide nucleic acid has a sequence of less than 15 nucleotides and has a sequence of 1, 2, 3, 4, 5, 6, 7, 8, 9, 10, 11, 12, 13, or 14 nucleotides (e.g., a sequence of 5 or 6 nucleotides in length). The RT template sequence may be after the PBS sequence in the 5′ to 3′ direction. In some embodiments, the RT template sequence of the extended guide nucleic acid has a length of greater than 65 nucleotides and may comprise about 50 or more nucleotides of heterology relative to the target site (e.g., target nucleic acid), followed by about 15 or more nucleotides of homology relative to the target site. In some embodiments, the RT template sequence of the extended guide nucleic acid is after the PBS sequence and the RT template sequence has a length of greater than 65 nucleotides with the sequence including more than 50 nucleotides of heterology relative to the target site, followed by more than 15 nucleotides of homology relative to the target site. Accordingly, in some embodiments, when the extended guide nucleic acid is reverse transcribed, the resulting newly transcribed sequence may hybridize and/or is configured to hybridize with the unnicked strand of the target site, which may thereby create a heteroduplex DNA with a large insertion into the newly synthesized strand. Upon repair of this mismatched DNA, the resultant repaired DNA may contain a large insertion (e.g., greater than 50 nucleotides) of DNA sequence. In some embodiments, the method may provide a large deletion (e.g., greater than 50 nucleotides) of DNA sequence. In some embodiments, the PBS and the 15 or more nucleotides of homology to the target site may comprise homology arms, which may serve to insert the heterology into the target site optionally using homology directed repair. The inserted DNA may correspond to any functional sequence of DNA such as, but not limited to: a functional transgene; a fragment of DNA that is inserted into a gene in a way that, when the gene is transcribed, would produce a hairpin RNA that is sufficient to silence homologous genes through RNAi; and/or one or more functional site-specific recombination sites, e.g. lox, frt, which could then be used in subsequent Cre or Flp mediated site-specific recombination processes. In some embodiments, an extended guide nucleic acid may be too large to produce using a PolIII promoter in vivo. In some embodiments, an extended guide nucleic acid may be operatively associated with and/or produced using a PolII promoter. In some embodiments, the DNA binding domain and/or DNA endonuclease may have a structure and/or be designed as described in Anzalone et al., Nature, 2019 December; 576 (7785): 149-157. In some embodiment, the DNA binding domain and/or DNA endonuclease is a CRISPR Cas polypeptide such as a Cas9 nickase or a similar nicking variant of another CRISPR Cas polypeptide such as, but not limited to, Cas12a.

In some embodiments, a polypeptide, polynucleotide, complex, composition, system, kit, and/or method of the present invention may be used to make and/or may make large edits (e.g., greater than 50 nucleotides in length) using homology directed repair. Exemplary large edits include, but are not limited to; large deletions, large inversions, inter-chromosomal recombinations, and/or intra-chromosomal recombinations. In some embodiments, a polypeptide, polynucleotide, complex, composition, system, kit, and/or method of the present invention may be used in and/or may be configured for use in a one cross editing (1XE) method and/or system in which modifying a target nucleic acid occurs during the step of haploid induction.

In some embodiments, two extended guide nucleic acids (e.g., pegRNAs) may be used. One or both of the extended guide nucleic acids may have a structure and/or be designed as described in Anzalone et al., Nature, 2019 December; 576 (7785): 149-157. The extended guide nucleic acids may comprise a primer binding site (PBS) optionally having a sequence of 1, 2, 3, 4, or 5 to 6, 7, 8, 9, 10, 11, 12, 13, 14, or 15 nucleotides and a reverse transcriptase template (RT template) sequence optionally having a sequence of 50 nucleotides or more. The RT template sequences of the two extended guide nucleic acids are complementary to each other and as such the polynucleotides that are respectively reverse transcribed from each the RT templates will be complementary to each other and will be able to hybridize with each other. This may allow for the intermediates that are produced by this system and/or method to join together two sections of DNA that are otherwise separated by more than 50 nucleotides, e.g. within a chromosome, or that are positioned on two separate pieces of DNA, e.g. on two different chromosomes. After repair of the intermediates, the resultant products may produce, depending on the design of the RT template, large deletions, large inversions, or inter-chromosomal recombinations. Since all of these products are produced by homology directed repair, the products may be predictably precise and/or reproducible. In some embodiments, the DNA binding domain and/or DNA endonuclease may have a structure and/or be designed as described in Anzalone et al., Nature, 2019 December; 576 (7785): 149-157. In some embodiment, the DNA binding domain and/or DNA endonuclease is a CRISPR Cas polypeptide such as a Cas9 nickase or a similar nicking variant of another CRISPR Cas polypeptide such as, but not limited to, Cas12a. In some embodiments, the DNA binding domain and/or DNA endonuclease is a Cas9 nuclease or a similar nuclease from another CRISPR Cas polypeptide such as, but not limited to, Cas12a. Using a nuclease (rather than a nickase) may facilitate the intra- or interchromosomal recombination processes through single-strand annealing of the more than 50 nucleotide 3′ overhangs that would be produced at each of the two target sites corresponding to the two pegRNA target nucleic acids.

In some embodiments, a polypeptide, polynucleotide, complex, composition, system, kit, and/or method of the present invention may be directed by homology to modify a target nucleic acid. In some embodiments, a polypeptide, polynucleotide, complex, composition, system, kit, and/or method of the present invention may be used to make and/or may make identical modifications (e.g., edits) in a target nucleic acid. In some embodiments, a polypeptide, polynucleotide, complex, composition, system, kit, and/or method of the present invention may be used to make and/or may make identical modifications (e.g., edits) in a target nucleic acid that are produced independently multiple times optionally in multiple germplasms.

In some embodiments, a DNA binding domain may be encoded by a polynucleotide, a DNA endonuclease may be encoded by a polynucleotide and a reverse transcriptase may be encoded by a polynucleotide. In some embodiments, the polynucleotide encoding the DNA binding domain, the polynucleotide encoding the DNA endonuclease and the polynucleotide encoding the reverse transcriptase may be comprised in the same or separate expression cassettes, optionally wherein when present in the same expression cassette, the polynucleotide encoding the DNA binding domain, the polynucleotide encoding the DNA endonuclease and the polynucleotide encoding the reverse transcriptase may be operably linked to a single promoter or they may be linked to two or more separate promoters in any combination.

In some embodiments, the expression cassettes of the invention may be comprised in one or more vectors. In some embodiments, the expression cassettes and/or the one or more vectors of the invention may comprise a guide RNA and/or an extended guide RNA.

In some embodiments, the methods of the invention may further comprises contacting the target nucleic acid with a second DNA binding domain, a second DNA endonuclease, and an RNA encoded template, optionally wherein the second DNA binding domain, the second DNA endonuclease, and the second reverse transcriptase are comprised in a complex.

In some embodiments, the second DNA binding protein may be a second DNA binding fusion protein comprising a second DNA binding protein domain fused (linked) to a peptide tag (e.g., a peptide repeat unit, an epitope or a multimerized epitope) and/or the second DNA endonuclease may be a second DNA endonuclease fusion protein comprising a second DNA endonuclease domain fused (linked) to a peptide tag (e.g., a peptide repeat unit, an epitope or a multimerized epitope), and the second reverse transcriptase may be a second reverse transcriptase fusion protein comprising a second reverse transcriptase domain fused (linked) to an affinity polypeptide that binds to the peptide tag, optionally wherein the target nucleic acid may be contacted with two or more second reverse transcriptase fusion proteins. In some embodiments, the methods of the invention may further comprise contacting the target nucleic acid with a guide nucleic acid. In some embodiments, the guide nucleic acid is linked to an RNA recruiting motif, and the second reverse transcriptase is a second reverse transcriptase fusion protein comprising a second reverse transcriptase domain fused (linked) to an affinity polypeptide that binds to the RNA recruiting motif, optionally wherein the target nucleic acid is contacted with two or more second reverse transcriptase fusion proteins. In some embodiments, the second DNA binding domain may be a CRISPR-Cas nuclease domain, a transcription activator-like effector (TALE) protein domain, and/or a zinc finger protein domain. In some embodiments, the second DNA endonuclease may be a CRISPR-Cas nuclease, and/or a Fok1 endonuclease. In some embodiments, the second DNA binding domain and the second DNA endonuclease may be comprised in a CRISPR-Cas nuclease. In some embodiments, the CRISPR-Cas nuclease may be a Cas9 nickase (nCas9), optionally wherein the Cas9 nickase is encoded by a polynucleotide that is optionally comprised in an expression cassette. In some embodiments, the second DNA binding domain may be encoded by a polynucleotide and the second DNA endonuclease may be encoded by a polynucleotide.

In some embodiments, a polynucleotide encoding the second DNA binding domain and a polynucleotide encoding the second DNA endonuclease may be comprised in the same or separate expression cassettes, optionally wherein when present in the same expression cassette, the polynucleotide encoding the second DNA binding domain and the polynucleotide encoding the second DNA endonuclease may be operably linked to a single promoter or to two or more separate promoters in any combination. In some embodiments, the expression cassettes of the invention may be comprised in one or more vectors, optionally wherein the expression cassettes and/or vectors of the invention may further comprise a guide RNA. In some embodiments, a guide nucleic acid and/or extended guide nucleic acid may be operably linked to a PolIII or PolII promoter.

In some embodiments, the methods of the invention may further comprise contacting a target nucleic acid with a 5′ flap endonuclease (FEN), optionally an FEN1 polypeptide. In some embodiments, the FEN may be overexpressed in the plant or plant cell. In some embodiments, the FEN may be fusion protein comprising an FEN domain fused to the DNA binding domain and/or the DNA endonuclease. In some embodiments, the DNA binding protein may be a DNA binding fusion protein comprising a DNA binding protein domain fused (linked) to a peptide tag (e.g., a peptide repeat unit, an epitope or a multimerized epitope) and/or the DNA endonuclease may be a DNA endonuclease fusion protein comprising a DNA endonuclease domain fused (linked) to a peptide tag (e.g., a peptide repeat unit, an epitope or a multimerized epitope) and the FEN may be an FEN fusion protein comprising an FEN domain fused (linked) to an affinity polypeptide that binds to the peptide repeat unit, optionally wherein the target nucleic acid is contacted with two or FEN fusion proteins, thereby recruiting the FEN to the DNA binding protein and/or DNA endonuclease, and the target nucleic acid.

In some embodiments of the invention, a reverse transcriptase (e.g., a first reverse transcriptase, a second reverse transcriptase and the like) may be fused to one or more ssRNA binding domains (RBDs).

In some embodiments of the invention, polynucleotides encoding DNA binding domains, DNA endonucleases, reverse transcriptase, flap endonucleases, extended guide nucleic acids, guide nucleic acids, expression cassettes and/or vectors may be codon optimized for expression in a plant, optionally wherein the polynucleotides may be codon optimized for expression in a dicot plant or for expression in a monocot plant.

In some embodiments, a peptide tag (e.g., a peptide repeat unit) may comprise 1 or 2 or more copies of a peptide repeat unit (e.g., an epitope, multimerized epitope) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, the peptide repeat unit may include, but is not limited to, a GCN4 peptide repeat unit (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope.

In some embodiments, an affinity polypeptide that binds to a peptide tag (e.g., a peptide repeat unit) may be an antibody, optionally wherein the antibody is a scFv antibody.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein and/or a synthetic RNA-aptamer and the aptamer ligand as the corresponding affinity polypeptide.

In some embodiments, the present invention provides a method of modifying a target nucleic acid in a plant cell, comprising contacting the nucleic acid with a DNA binding domain and a DNA endonuclease domain targeted to a first site on the target nucleic acid and the same or a different DNA binding domain and DNA endonuclease domain targeted to a second site on the target nucleic acid, wherein the first site and the second site are proximal to one another on the same (nontarget) strand, thereby nicking the target nucleic acid at the first and second site; a reverse transcriptase; and a nucleic acid encoded repair template encoding a modification to be incorporated into the target nucleic acid, thereby modifying the target nucleic acid in the plant.

In some embodiments, a method of modifying a target nucleic acid in a plant cell is provided, the method comprising: contacting the target nucleic acid with (a) a CRISPR-Cas nuclease comprising a first DNA binding domain and a first DNA endonuclease (a nickase); (b) a reverse transcriptase; (c) a CRISPR RNA (crRNA) comprising a spacer having substantial homology to a first site on the target nucleic acid; (d) a trans-activating crRNA (tracrRNA) that interacts (recruits/binds) with the crRNA and the CRISPR-Cas nuclease; and (e) a nucleic acid encoded repair template (e.g., an RNA encoded repair template) comprising a primer binding site and an template encoding the modification to be incorporated into the target nucleic acid, wherein the tracrRNA comprises a sequence at the 5′ or 3′ end that is complementary to a sequence at the 5′ end or 3′ end of the reverse transcriptase template, thereby modifying the target nucleic acid. In some embodiments, the methods of the invention further comprise contacting the target nucleic acid with two or more crRNAs, two or more tracrRNAs, two or more nucleic acid encoded repair templates and/or two or more CRISPR-Cas nucleases. In some embodiments, a method of the invention further comprises contacting the target nucleic acid (e.g., target DNA) with a second crRNA comprising a spacer having substantial homology to a second site on the target nucleic acid that is proximal to and on the same strand (non-target strand) as the first site and a second tracrRNA, wherein the second tracrRNA may or may not comprise a sequence at the 5′ or 3′ end that is complementary to a sequence at the 5′end or 3′ end of the reverse transcriptase template (for a double nick to remove the wild type nucleic acid). In some embodiments, a method of the invention further comprises contacting the target nucleic acid (e.g., target DNA) with a second crRNA comprising a spacer having substantial homology to a third site on the target nucleic acid that is on a different strand from the first site (e.g., for improved mismatch repair).

In some embodiments, the present invention provides a method of modifying a target nucleic acid in a plant cell, the method comprising: contacting the target nucleic acid with (a) a CRISPR-Cas nuclease comprising a first DNA binding domain and a first DNA endonuclease (a nickase); (b) a reverse transcriptase; (c) a CRISPR RNA (crRNA) comprising a spacer having substantial homology to a first site on the target nucleic acid; (d) a trans-activating crRNA (tracrRNA) that interacts (recruits/binds) with the crRNA and the CRISPR-Cas nuclease; and a nucleic acid encoded repair template (e.g., an RNA encoded repair template) comprising a primer binding site and a template encoding the modification to be incorporated into the target nucleic acid, thereby modifying the target nucleic acid. In some embodiments, the methods of the invention further comprise contacting the target nucleic acid with two or more crRNAs, two or more tracrRNAs and/or two or more CRISPR-Cas nucleases. In some embodiments, a method of the invention further comprises contacting the target nucleic acid (e.g., target DNA) with a second crRNA comprising a spacer having substantial homology to a second site on the target nucleic acid that is proximal to and on the same strand (non-target strand) as the first site and a second tracrRNA that interacts (recruits/binds) with the second crRNA and either the first CRISPR-Cas nuclease or a different CRISPR-Cas nuclease thereby providing a double nick for removing the wild type nucleic acid. In some embodiments, a method of the invention further comprises contacting the target nucleic acid (e.g., target DNA) with a third crRNA comprising a spacer having substantial homology to a third site on the target nucleic acid that is on a different strand (target strand) from the first site and a third tracrRNA that interacts (recruits/binds) with the third crRNA and either the first CRISPR-Cas nuclease or a different CRISPR-Cas nuclease thereby improving mismatch repair.

In some embodiments, a method of modifying a target nucleic acid in a plant cell is provided, the method comprising: contacting the target nucleic acid with (a) a CRISPR-Cas nuclease comprising a first DNA binding domain and a first DNA endonuclease (e.g., a nickase); (b) a reverse transcriptase; (c) a CRISPR RNA (crRNA) guide that interacts (recruits/binds) with the CRISPR-Cas nuclease and comprises a spacer having substantial homology to a first site on the target nucleic acid; (e) a nucleic acid encoded repair template (e.g., an RNA encoded repair template) comprising a primer binding site and an RNA template (that encodes the modification to be incorporated into the target nucleic acid), wherein the crRNA comprises a sequence at its 5′ end or 3′ end that is complementary to the primer binding site, thereby modifying the target nucleic acid. In some embodiments, a method of the invention further comprises contacting the target nucleic acid with two or more crRNAs, two or more nucleic acid encoded repair templates and/or two or more CRISPR-Cas nucleases. In some embodiments, a method of the invention further comprises contacting the target nucleic acid (e.g., target DNA) with a second crRNA that interacts (recruits/binds) with the either the first CRISPR-Cas nuclease or a different CRISPR-Cas nuclease and comprises a spacer having substantial homology to a second site on the target nucleic acid that is proximal to and on the same strand (non-target strand) as the first site, thereby providing a double nick for removing the wild type nucleic acid. In some embodiments, a method of the invention further comprises contacting the target nucleic acid (e.g., target DNA) with a third crRNA that interacts (recruits/binds) with either the first CRISPR-Cas nuclease or a different CRISPR-Cas nuclease and comprises a spacer having substantial homology to a third site on the target nucleic acid that is on a different strand (target strand) from the first site, thereby improving mismatch repair.

In some embodiments, a method of modifying a target nucleic acid in a plant cell is provided, the method comprising contacting the target nucleic acid with (a) a CRISPR-Cas nuclease comprising a first DNA binding domain and a first DNA endonuclease (e.g., a nickase); (b) a reverse transcriptase; (c) an extended guide nucleic acid comprising a sequence that interacts that interacts (recruits/binds) with the CRISPR-Cas nuclease and a spacer having substantial homology to a first site on the target nucleic acid (e.g., CRISPR RNA (crRNA) (a first crRNA) and/or tracrRNA+crRNA (sgRNA)) and a nucleic acid encoded repair template (e.g., an RNA encoded repair template) comprising a primer binding site and an RNA template (that encodes the modification to be incorporated into the target nucleic acid), thereby modifying the target nucleic acid. In some embodiments, a method of the invention further comprises contacting the target nucleic acid with two or more extended guide nucleic acids and/or two or more CRISPR-Cas nucleases. In some embodiments, a method of the invention further comprises contacting the target nucleic acid (e.g., target DNA) with a crRNA (e.g., a second crRNA) that interacts (recruits/binds) with the either the first CRISPR-Cas nuclease or a different CRISPR-Cas nuclease and comprises a spacer having substantial homology to a second site on the target nucleic acid that is proximal to and on the same strand (non-target strand) as the first site, thereby providing a double nick for removing the wild type nucleic acid. In some embodiments, a method of the invention further comprises contacting the target nucleic acid (e.g., target DNA) with a second crRNA (e.g. a third crRNA) that interacts (recruits/binds) with either the first CRISPR-Cas nuclease or a different CRISPR-Cas nuclease and comprises a spacer having substantial homology to a third site on the target nucleic acid that is on a different strand (target strand) from the first site, thereby improving mismatch repair.

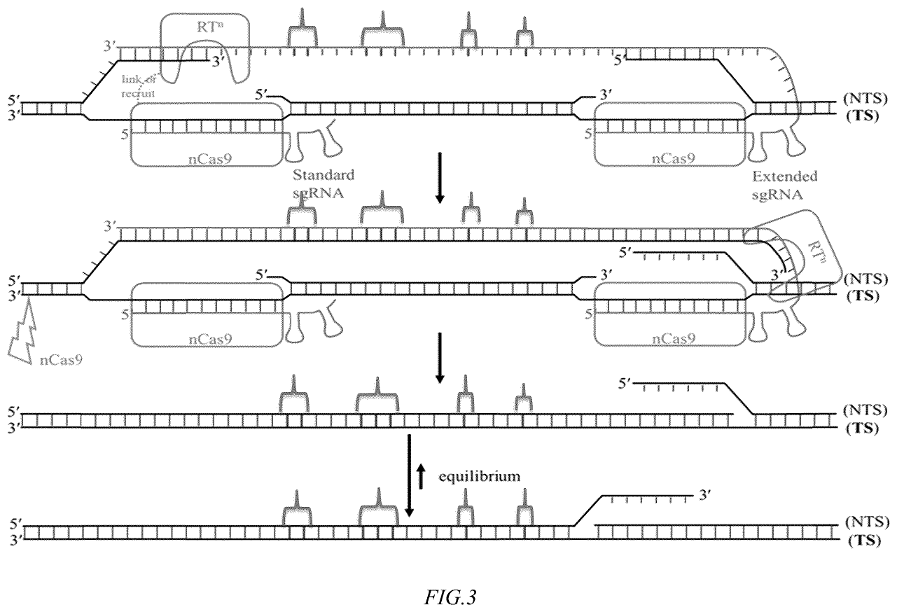

In some embodiments, the invention provides a method of modifying a target nucleic acid in a plant cell, the method comprising contacting the target nucleic acid with (a) a first CRISPR-Cas nuclease (a nickase) comprising a first DNA binding domain and a first DNA endonuclease; (b) an extended guide nucleic acid comprising a CRISPR RNA (crRNA) comprising a spacer having substantial homology to a first site on the target nucleic acid, a trans-activating crRNA (tracrRNA) that recruits the first CRISPR-Cas nuclease and an RNA template comprising the modification to be incorporated into the target nucleic acid, wherein the first CRISPR-Cas nuclease nicks the target nucleic acid at a first site (on the non-target strand); (c) a second CRISPR-Cas nuclease (e.g., a nickase) comprising a first DNA binding domain and a first DNA endonuclease (e.g., a nickase); (d) a guide nucleic acid comprising a CRISPR RNA (crRNA) comprising a spacer having substantial homology to a second site on the target nucleic acid that is proximal to (and on the same strand as) the first site on the target nucleic acid, a trans-activating crRNA (tracrRNA) that recruits the second CRISPR-Cas nuclease, thereby nicking the target nucleic acid at the second site (on the non-target strand); and (e) a reverse transcriptase fused or recruited to the first CRISPR Cas-nuclease and/or the second CRISPR Cas-nuclease, thereby modifying the target nucleic acid. See e.g., FIG. 3.

In some embodiments, a method of releasing a portion of a double stranded nucleic acid is provided, comprising: (a) targeting a first DNA endonuclease to a first site of the nucleic acid; (b) making a nick at in a first strand of the nucleic acid at the first site; (c) targeting the first DNA endonuclease or a second DNA endonuclease to a second site on the first strand; and (d) making a nick in the first strand at the second site, wherein the portion of the first strand of the nucleic acid between the first site and second site can be released from the nucleic acid. In some embodiments, the method further comprises contacting the nucleic acid with a reverse transcriptase. In some embodiments, the method further comprises contacting the nucleic acid with a reverse transcriptase template. In some embodiments, a transcriptase template may comprise a sequence substantially similar to the released portion of the nucleic acid and additionally comprises at least one nucleotide insertion, deletion or substitution. In some embodiments, the reverse transcriptase template may replace the released portion and become part of the double stranded nucleic acid.

In some embodiments, the invention provides an insertion of one or more nucleotide(s) in an organism (e.g., a plant). The insertion may comprise a recombination site or a whole gene at a specific genomic locus in the organism. In some embodiments, a reverse transcriptase template within an extended guide nucleic acid includes the insertion sequence such as, but not limited to, a recombination site (e.g., a wild type or mutated loxP, FRT, RS, attP and attB site) or a coding sequence of a gene and/or a regulatory element (e.g., promoter, 5′UTR sequence, and/or 3′UTR sequence). Exemplary recombination site sequences include, but are not limited to, those listed in Table 1. In some embodiments of the invention, the 3′ end of a guide nucleic acid (e.g., a sgRNA) may comprise a sequence that is complimentary a region comprising the target nucleic acid, optionally the 3′ end of a target nucleic acid. In some embodiments of the invention, the 3′ end of a guide nucleic acid (e.g., a sgRNA) may comprise a microhomology region (e.g., a small region of homology such as 5-25 nucleotides in length) that binds to the 3′ end of a target nucleic acid, which may optionally provide microhomolgy mediated end joining (MMEJ) and/or a repair mechanism.

TABLE 1
Exemplary recombination site sequences.

| Recombination | |
|---|---|
| site | Sequence |
| loxP | 5′-ATAACTTCGTATA ATGTATGC TATACGAAGTTAT-3′ |
| | (SEQ ID NO: 148) |
| lox75 | 5′-ATAACTTCGTATA ATGTATGC TATACGcccggta-3′ |
| | (SEQ ID NO: 149) |
| lox76 | 5′-taccgggCGTATA ATGTATGC TATACGAAGTTAT-3′ |
| | (SEQ ID NO: 150) |
| lox66 | 5′-taccgTTCGTATA ATGTATGC TATACGAAGTTAT-3′ |
| | (SEQ ID NO: 151) |
| lox71 | 5′-ATAACTTCGTATA ATGTATGC TATACGAAcggta-3′ |
| | (SEQ ID NO: 152) |
| lox78 | 5′-taccgggCGTATA ATGTATGC TATACGcccggta-3′ |
| | (SEQ ID NO: 153) |
| lox72 | 5′-taccgTTCGTATA ATGTATGC TATACGAAcggta-3′ |
| | (SEQ ID NO: 154) |
| FRT | 5′-GAAGTTCCTATTC TCTAGAAA GTATAGGAACTTC-3′ |
| | (SEQ ID NO: 155) |
| RS | 5′-TTGATGAAAGAA TACGTTA TTCTTTCATCAA-3′ |
| | (SEQ ID NO: 156) |
| phiC31-attP | 5′-CCCCAACTGGGGTAACCTTTGAGTTCTCTCAGTTGGGGG-3′ |
| | (SEQ ID NO: 157) |
| phiC31-attB | 5′-GTGCCAGGGCGTGCCCTTGGGCTCCCCGGGCGCG-3′ |
| | (SEQ ID NO: 158) |
| Bxb1-attP | 5′-GGTTTGTCTGGTCAACCACCGCGGTCTCAGTGGTGTACGGTACA |
| | AACC-3′(SEQ ID NO: 159) |
| Bxb1-attB | 5′-CCGGCTTGTCGACGACGGCGGTCTCCGTCGTCAGGATCATCC-3′ |
| | (SEQ ID NO: 160) |

In some embodiments, polynucleotides, nucleic acid constructs, expression cassettes and vectors may be provided for carrying out the methods of the invention. Thus, in some embodiments an expression cassette is provided that is codon optimized for expression in a plant, comprising 5′ to 3′ (a) polynucleotide encoding a plant specific promoter sequence (e.g. ZmUbi1, MtUb2, RNA polymerase II (Pol II)), (b) a plant codon-optimized polynucleotide encoding a CRISPR-Cas nuclease (e.g. nCas9, dCas9, Cpf1 (Cas12a), dCas12a and the like), (c) a linker sequence; and (d) a plant codon-optimized polynucleotide encoding a reverse transcriptase.

In some embodiments of the invention, a reverse transcriptase may be fused to one or more ssRNA binding domains (RBDs).

In some embodiments, polypeptides of the invention may be fusion proteins comprising one or more polypeptides linked to one another via a linker. In some embodiments, the linker may be an amino acid or peptide linker. In some embodiments, a peptide linker may be about 2 to about 100 amino acids (residues) in length. In some embodiments, a peptide linker may be a GS linker.

In some embodiments, the invention provides an expression cassette that is codon optimized for expression in a plant, comprising: (a) a polynucleotide encoding a plant specific promoter sequence (e.g. ZmUbi1, MtUb2), and (b) an extended guide nucleic acid, wherein the extended guide nucleic acid comprises an extended portion comprising at its 3′ end a primer binding site and an edit to be incorporated into the target nucleic acid (e.g., reverse transcriptase template), optionally wherein the extended guide nucleic acid is comprised in an expression cassette, optionally wherein the extended guide nucleic acid is operably linked to a Pol II promoter.

In some embodiments, a plant specific promoter may be associated with an intron or may be a promoter region comprising an intron (e.g., ZmUbi1 comprising an intron; MtUb2 comprising an intron).

In some embodiments, an expression cassette of the invention may be codon optimized for expression in a dicot plant or for expression in a monocot plant. In some embodiments, the expression cassettes of the invention may be used in a method of modifying a target nucleic acid in a plant or plant cell, the method comprising introducing one or more expression cassettes of the invention into a plant or plant cell, thereby modifying the target nucleic acid in the plant or plant cell to produce a plant or plant cell comprising the modified target nucleic acid. In some embodiments, the method may further comprise regenerating the plant cell comprising the modified target nucleic acid to produce a plant comprising the modified target nucleic acid.

In some embodiments, the present invention provides a nucleic acid molecule comprising (a) a sequence that interacts (e.g., binds, recruits) with a CRISPR-Cas nuclease (tracrRNA), (b) a sequence that directs the CRISPR-Cas nuclease to a target nucleic acid (e.g., a crRNA), and (c) a sequence encoding a template for introducing a modification into the target nucleic acid, or (a) a sequence that that interacts (e.g., binds, recruits) with a CRISPR-Cas nuclease and directs the CRISPR-Cas nuclease to a target nucleic acid (crRNA) and (b) a sequence encoding a template for introducing a modification into the target nucleic acid.

In some embodiments of the invention, a CRISPR-Cas nuclease, a DNA binding domain, and/or a DNA endonuclease may be from a Type I CRISPR-Cas system, a Type II CRISPR-Cas system, a Type III CRISPR-Cas system, a Type IV CRISPR-Cas system or a Type V CRISPR-Cas system. In some embodiments, the CRISPR-Cas nuclease is from a Type II CRISPR-Cas system or a Type V CRISPR-Cas system. In some embodiments, a CRISPR-Cas effector protein may be a Type II CRISPR-Cas effector protein, for example, a Cas9 effector protein. In some embodiments, a CRISPR-Cas effector protein may be Type V CRISPR-Cas effector protein, for example, a Cas12 effector protein.

In some embodiments of the invention, a CRISPR-Cas nuclease, a DNA binding domain, and/or a DNA endonuclease may be a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas nuclease may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c nuclease.

In some embodiments, a CRISPR-Cas nuclease, a DNA binding domain, and/or a DNA endonuclease may be a Cas9 nickase or a Cas12a nickase.

In some embodiments, a polynucleotide encoding a DNA binding polypeptide or domain, a polynucleotide encoding a DNA endonuclease polypeptide or domain, a polynucleotide encoding a reverse transcriptase polypeptide or domain, and/or a polynucleotide encoding a flap endonuclease polypeptide or domain may be operably linked to at least one regulatory sequence, optionally, wherein the at least one regulatory sequence may be codon optimized for expression in a plant. In some embodiments, the at least one regulatory sequence may be, for example, a promoter, an operon, a terminator, or an enhancer. In some embodiments, the at least one regulatory sequence may be a promoter. In some embodiments, the regulatory sequence may be an intron. In some embodiments, the at least one regulatory sequence may be, for example, a promoter operably associated with an intron or a promoter region comprising an intron. In some embodiments, the at least one regulatory sequence may be, for example a ubiquitin promoter and its associated intron (e.g., Medicago truncatula and/or Zea mays and their associated introns). In some embodiments, the at least one regulatory sequence may be a terminator nucleotide sequence and/or an enhancer nucleotide sequence.

In some embodiments, the present invention provides a polynucleotide encoding a DNA binding polypeptide or domain, a polynucleotide encoding an endonuclease polypeptide or domain, a polynucleotide encoding a reverse transcriptase polypeptide or domain, and/or a polynucleotide encoding a flap endonuclease polypeptide or domain operably associated with one or more promoter regions, wherein one or more of the promoter regions may comprises an intron, optionally wherein the promoter region may be a ubiquitin promoter and intron (e.g., a Medicago or a maize ubiquitin promoter and intron, e.g., SEQ ID NOs: 1 or 2). In some embodiments, the CRISPR-Cas nuclease operably associated with a promoter region comprising an intron may be codon optimized for expression in a plant.

A CRISPR-Cas nuclease useful with this invention can include, but is not limited, to Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas nuclease may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas nuclease useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas nuclease having a mutation in its nuclease active site, and therefore, no longer comprising nuclease activity, is commonly referred to as “dead,” e.g., dCas such as dCas9. In some embodiments, a CRISPR-Cas nuclease domain or polypeptide having a mutation in its nuclease active site may have impaired activity or reduced activity as compared to the same CRISPR-Cas nuclease without the mutation, e.g., a nickase, e.g., Cas9 nickase, Cas12a nickase.

A CRISPR Cas9 polypeptide or CRISPR Cas9 domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. In some embodiments, a CRISPR-Cas nuclease may be a Cas9 polypeptide or domain thereof and optionally may have a nucleotide sequence of any one of SEQ ID NOs: 3-13 and/or an amino acid sequence of any one of SEQ ID NOs: 14-15.

In some embodiments, the CRISPR-Cas nuclease may be a Cas9 polypeptide derived from Streptococcus pyogenes and recognizes the PAM sequence motif NGG, NAG, NGA (Mali et al, Science 2013; 339 (6121): 823-826). In some embodiments, the CRISPR-Cas nuclease may be a Cas9 polypeptide derived from Streptococcus thermophiles and recognizes the PAM sequence motif NGGNG and/or NNAGAAW (W=A or T) (See, e.g., Horvath et al, Science, 2010; 327 (5962): 167-170, and Deveau et al, J Bacteriol 2008; 190 (4): 1390-1400). In some embodiments, the CRISPR-Cas nuclease may be a Cas9 polypeptide derived from Streptococcus mutans and recognizes the PAM sequence motif NGG and/or NAAR (R=A or G) (See, e.g., Deveau et al, J BACTERIOL 2008; 190 (4): 1390-1400). In some embodiments, the CRISPR-Cas nuclease may be a Cas9 polypeptide derived from Streptococcus aureus and recognizes the PAM sequence motif NNGRR (R=A or G). In some embodiments, the CRISPR-Cas nuclease may be a Cas9 protein derived from S. aureus, which recognizes the PAM sequence motif N GRRT (R=A or G). In some embodiments, the CRISPR-Cas nuclease may be a Cas9 polypeptide derived from S. aureus, which recognizes the PAM sequence motif N GRRV (R=A or G). In some embodiments, the CRISPR-Cas nuclease may be a Cas9 polypeptide that is derived from Neisseria meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the CRISPR-Cas nuclease may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

A Type V CRISPR-Cas nuclease useful with embodiments of the invention may be any Type V CRISPR-Cas nuclease. A Type V CRISPR-Cas nuclease useful with this invention as an effector protein can include, but is not limited, to Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c nuclease. In some embodiments, a Type V CRISPR-Cas nuclease polypeptide or domain useful with embodiments of the invention may be a Cas12a polypeptide or domain. In some embodiments, a Type V CRISPR-Cas nuclease or domain useful with embodiments of the invention may be a nickase, optionally, a Cas12a nickase.

In some embodiments, the CRISPR-Cas nuclease may be derived from Cas12a, which is a Type V Clustered Regularly Interspaced Short Palindromic Repeats (CRISPR)-Cas nuclease. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas 12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a polypeptide or CRISPR Cas12a domain useful with this invention may be any known or later identified Cas12a nuclease (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

In some embodiments, a CRISPR-Cas nuclease may be optimized for expression in an organism, for example, in an animal, a plant, a fungus, an archaeon, or a bacterium. In some embodiments, a CRISPR-Cas nuclease (e.g., Cas12a polypeptide/domain or a Cas9 polypeptide/domain) may be optimized for expression in a plant. In some embodiments, a Cas12a polypeptide/domain that may be optimized according to the present invention can include, but is not limited to, the amino acid sequence of any one of SEQ ID NOs: 16-32 (e.g., SEQ ID NOs: 16, 17, 18, 19, 20, 21, 22, 23, 24, 25, 26, 27, 28, 29, 30, 31, or 32), or a polynucleotide encoding the same such as, but not limited to, the polynucleotide of any one of SEQ ID NOs: 33-35.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. In some embodiments, the guide nucleic acid comprises DNA. In some embodiments, the guide nucleic acid comprises RNA (e.g., is a guide RNA). The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer. In some embodiments, as described herein, a guide RNA may include a template for editing and a primer binding site. In some embodiments, a guide RNA may include a region or sequence on its 5′ end or 3′ end that is complementary to an editing template (a reverse transcriptase template), thereby recruiting the editing template to the target nucleic acid.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas nuclease encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35 (Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 23 nucleotides in length.

In some embodiments, the 5′ region of a spacer sequence of a guide nucleic acid (e.g., guide RNA) may be identical to a target DNA, while the 3′ region of the spacer may be substantially complementary to the target DNA (e.g., Type V CRISPR-Cas), or the 3′ region of a spacer sequence of a guide nucleic acid may be identical to a target DNA, while the 5′ region of the spacer may be substantially complementary to the target DNA (e.g., Type II CRISPR-Cas), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA. A recruiting guide RNA further comprises one or more recruiting motifs as described herein, which may be linked to the 5′ end of the guide or the 3′ end or it may be inserted into the recruiting guide nucleic acid (e.g., within the hairpin loop).

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refer to a region of an organism's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid (e.g., guide RNA) of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

| | |
|---|---|
| 5′-NNNNNNNNNNNNNNNNNNN-3′ | RNA Spacer (SEQ ID NO: 36) |
| \|\|\|\|\|\|\|\|\|\|\|\|\|\|\|\|\|\|\|\| | |
| 3′AAANNNNNNNNNNNNNNNNNNN-5′ | Target strand (SEQ ID NO: 37) |
| \|\|\|\| | |
| 5′TTTNNNNNNNNNNNNNNNNNNN-3′ | Non-target strand (SEQ ID NO: 38 |

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

Fusion proteins of the invention may comprise a DNA binding domain, DNA endonuclease, guide nucleic acid, or reverse transcriptase fused to a peptide tag or an affinity polypeptide. In some embodiments, a DNA binding domain is fused to a peptide tag or an affinity polypeptide that interacts with the peptide tag, as known in the art, for use in recruiting the DNA binding domain to the target nucleic acid, and/or an DNA endonuclease is fused to a peptide tag or an affinity polypeptide that interacts with the peptide tag, as known in the art, for use in recruiting the DNA endonuclease to the target nucleic acid. In some embodiments, a method of recruiting may comprise a guide nucleic acid linked to an RNA recruiting motif and a reverse transcriptase fused to an affinity polypeptide capable of interacting with the RNA recruiting motif, thereby recruiting the reverse transcriptase to the target nucleic acid. Alternatively, chemical interactions may be used to recruit a polypeptide (e.g., a reverse transcriptase) to a target nucleic acid.

As described herein, a “peptide tag” may be employed to recruit one or more polypeptides. A peptide tag may be any polypeptide that is capable of being bound by a corresponding affinity polypeptide. A peptide tag may also be referred to as an “epitope” and when provided in multiple copies, a “multimerized epitope.” Example peptide tags can include, but are not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. In some embodiments, a peptide tag may also include phosphorylated tyrosines in specific sequence contexts recognized by SH2 domains, characteristic consensus sequences containing phosphoserines recognized by 14-3-3 proteins, proline rich peptide motifs recognized by SH3 domains, PDZ protein interaction domains or the PDZ signal sequences, and an AGO hook motif from plants. Peptide tags are disclosed in WO2018/136783 and U.S. Patent Application Publication No. 2017/0219596, which are incorporated by reference for their disclosures of peptide tags. Peptide tags that may be useful with this invention can include, but are not limited to, SEQ ID NO: 39 and SEQ ID NO: 40. An affinity polypeptide useful with peptide tags includes, but is not limited to, SEQ ID NO: 41.

A peptide tag may comprise or be present in one copy or in 2 or more copies of the peptide tag (e.g., multimerized peptide tag or multimerized epitope) (e.g., about 2, 3, 4, 5, 6, 7, 8, 9, 10, 11, 12, 13, 14, 15, 16, 17, 18, 9, 20, 21, 22, 23, 24, or 25 or more peptide tags). When multimerized, the peptide tags may be fused directly to one another or they may be linked to one another via one or more amino acids (e.g., 1, 2, 3, 4, 5, 6, 7, 8, 9, 10, 11, 12, 13, 14, 15, 16, 17, 18, 19, 20 or more amino acids, optionally about 3 to about 10, about 4 to about 10, about 5 to about 10, about 5 to about 15, or about 5 to about 20 amino acids, and the like, and any value or range therein. Thus, in some embodiments, a CRISPR-Cas nuclease of the invention may comprise a CRISPR-Cas nuclease domain fused to one peptide tag or to two or more peptide tags, optionally wherein the two or more peptide tags are fused to one another via one or more amino acid residues. In some embodiments, a peptide tag useful with the invention may be a single copy of a GCN4 peptide tag or epitope or may be a multimerized GCN4 epitope comprising about 2 to about 25 or more copies of the peptide tag (e.g., about 2, 3, 4, 5, 6, 7, 8, 9, 10, 11, 12, 13, 14, 15, 16, 17, 18, 19, 20, 21, 22, 23, 24, 25 or more copies of a GCN4 epitope or any range therein).

Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22 (4):413-420 (2013)), U.S. Pat. No. 9,982,053, each of which are incorporated by reference in their entireties for the teachings relevant to affibodies, anticalins, monobodies and/or DARPins.

In some embodiments, a guide nucleic acid may be linked to an RNA recruiting motif, and a polypeptide to be recruited (e.g., a reverse transcriptase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., reverse transcriptase). In some embodiments, two or more polypeptides may be recruited to a guide nucleic acid, thereby contacting the target nucleic acid with two or more polypeptides.

In some embodiments of the invention, a guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF). Exemplary RNA recruiting motifs and corresponding affinity polypeptides that may be useful with this invention can include, but are not limited to, SEQ ID NOs: 42-52.

In some embodiments, the components for recruiting polypeptides and nucleic acids may include those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together; e.g. dihyrofolate reductase (DHFR)).

In some embodiments, a peptide tag may be fused to a CRISPR-Cas polypeptide or domain. In some embodiments, a peptide tag may be fused or linked to the C-terminus of a CRISPR-Cas nuclease to form a CRISPR-Cas fusion protein. In some embodiments, a peptide tag may be fused or linked to the N-terminus of a CRISPR-Cas nuclease to form a CRISPR-Cas fusion protein. In some embodiments, a peptide tag may be fused within a CRISPR-Cas nuclease (e.g., a peptide tag may be in a loop region of a CRISPR-Cas effector protein).

In some embodiments, when a peptide tag comprises more than one peptide tag, the quantity and spacing of each peptide tag may be optimized to maximize occupation of the peptide tags and minimize steric interference of, for example, polypeptide portions, with each other.

An “affinity polypeptide” (e.g., “recruiting polypeptide”) refers to any polypeptide that is capable of binding to its corresponding peptide tag, peptide tag, or RNA recruiting motif. An affinity polypeptide for a peptide tag may be, for example, an antibody and/or a single chain antibody that specifically binds the peptide tag, respectively. In some embodiments, an antibody for a peptide tag may be, but is not limited to, a scFv antibody. In some embodiments, an affinity polypeptide may be fused or linked to the N-terminus of a reverse transcriptase. In some embodiments, the affinity polypeptide is stable under the reducing conditions of a cell or cellular extract.

The nucleic acid constructs of the invention and/or guide nucleic acids may be comprised in one or more expression cassettes as described herein. In some embodiments, a nucleic acid construct of the invention may be comprised in the same or in a separate expression cassette or vector from that comprising a guide nucleic acid and/or a recruiting guide nucleic acid.

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same but which have not been codon optimized for expression in a plant.

In some embodiments, the invention provides cells (e.g., plant cells, animal cells, bacterial cells, archaeon cells, and the like) comprising one or more polynucleotides, guide nucleic acids, nucleic acid constructs, expression cassettes or vectors of the invention.

When used in combination with guide nucleic acids, the nucleic acid constructs of the invention (and expression cassettes and vectors comprising the same) may be used to modify a target nucleic acid. A target nucleic acid may be contacted with a nucleic acid construct of the invention and/or expression cassettes and/or vectors comprising the same prior to, concurrently with or after contacting the target nucleic acid with the guide nucleic acid. In some embodiments, the nucleic acid constructs of the invention and a guide nucleic acid may be comprised in the same expression cassette or vector and therefore, a target nucleic acid may be contacted concurrently with the nucleic acid constructs of the invention and guide nucleic acid. In some embodiments, the nucleic acid constructs of the invention and a guide nucleic acid may be in different expression cassettes or vectors and thus, a target nucleic acid may be contacted with the nucleic acid constructs of the invention prior to, concurrently with, or after contact with a guide nucleic acid.

In some embodiments, after contacting a target nucleic acid with a polypeptide, composition, complex (e.g., an assembled ribonucleoprotein complex), nucleic acid construct, expression cassette, and/or vector of the present invention, the cell and/or organism comprising the target nucleic acid may be exposed to and/or provided in an environment having a temperature of greater than 25° C. for a period of time (e.g., 1, 2, 3, 4, 5, 6, 7, 8, 9, 10, 11, 12, 13, 14, 15, 20, 30, 40, 50, 60 or more minutes, hours, or days). In some embodiments, the cell and/or organism is exposed to (e.g., provided, incubated, cultured, grown, or the like in an environment at) a temperature in a range of about 26° C., 28° C., 30° C., or 32° C. to about 34° C., 36° C., 38° C., 40° C., or 42° C. for a period of time (e.g., 1, 2, 3, 4, 5, 6, 7, 8, 9, 10, 11, 12, 13, 14, 15, 20, 30, 40, 50, 60 or more minutes, hours, or days). Exposing the cell and/or organism to a temperature of greater than 25° C. for a period of time may increase editing efficiency optionally by increasing reverse transcriptase activity and/or breaking RNA secondary structure elements in the extended guide nucleic acid. In some embodiments, exposing the cell and/or organism to a temperature of greater than 25° C. may improve performance of a polypeptide, composition, complex (e.g., an assembled ribonucleoprotein complex), nucleic acid construct, expression cassette, and/or vector of the present invention. In some embodiments, the organism is a plant tissue and after contacting and/or transforming a plant cell of the plant tissue with a polypeptide, composition, complex (e.g., an assembled ribonucleoprotein complex), nucleic acid construct, expression cassette, and/or vector of the present invention, the plant tissue is incubated at a temperature of greater than 25° C. for a period of time (e.g., 1, 2, 3, 4, 5, 6, 7, 8, 9, 10, 11, 12, 13, 14, 15, 20, 30, 40, 50, 60 or more minutes, hours, or days). In some embodiments, a method of the present invention comprises exposing a cell and/or organism to two or more different temperatures. For example, before, during, and/or after contacting and/or transforming a cell of an organism with a polypeptide, composition, complex (e.g., an assembled ribonucleoprotein complex), nucleic acid construct, expression cassette, and/or vector of the present invention, the cell is exposed to a first temperature of about 25° C. or less and then a second temperature of greater than 25° C. (e.g., about 26° C. to about 42° C.) or vice versa. In some embodiments, the first temperature is before and/or during the contacting and/or transforming step and the second temperature is after the contacting and/or transforming step.

According to some embodiments of the present invention, a polypeptide, polynucleotide, complex, composition, system, kit, and/or method of the present invention may be used and/or configured to modify (e.g., edit) one or more locus (loci) in a genome to alter gene function. In some embodiments, this may be achieved through a modification to a promoter, enhancer, 5′ UTR, exon, intron, 3′ UTR, terminator, miRNA binding site, and/or other functional element and/or junction between such elements. In some embodiments, a polypeptide, polynucleotide, complex, composition, system, kit, and/or method of the present invention may be used and/or configured to provide one or more targeted promoter sequence change(s). Targeted promoter sequence changes could be designed in a rational way to increase or decrease gene expression at any spatio-temporal point through insertion or deletion of known regulatory sequences. Targeted sequence changes may also be used in non-rational designs to develop allelic diversity that is screened to phenotypically determine a favorable allele. In some embodiments, a method of the present invention comprises generating allelic diversity to be screened such as by targeting a promoter region in a promoter bashing type approach. A library may be generated that includes 2 to 5, 10, 25, 50, 100, 200, 300, 400, 500, or more extended guide nucleic acids that are targeted against a gene promoter or coding sequence, which may aid in introducing and/or which may introduce a large amount of allelic variation that may be useful for screening for optimized phenotypes.

In some embodiments, a polynucleotide, complex, composition, system, kit, and/or method of the present invention comprises a crRNA (e.g., 1, 2, 3, 4, or more crRNA(s)) that has an extended 3′ extension, and the crRNA may aid in and/or be configured to aid in creating allelic diversity in an organism. The crRNA may be delivered with a DNA binding domain and/or DNA endonuclease (e.g., a CRISPR Cas polypeptide) or may be delivered separately. In some embodiments, the crRNA may be delivered assembled and/or in the same complex as a DNA binding domain and/or DNA endonuclease (e.g., a CRISPR Cas polypeptide). In some embodiments, a crRNA and a DNA binding domain and/or DNA endonuclease are delivered separately to a cell (e.g., a plant cell). In some embodiments, a first organism (e.g., a first plant or line (A)) may be transformed with a DNA binding domain and/or DNA endonuclease (e.g., a CRISPR Cas polypeptide) and optionally 0, 1, 2, or 3 crRNA(s), and a second organism (e.g., a second plant or line (B)) may be transformed with one or more crRNA(s). The first organism (e.g., line A) may be modified (e.g., edited) at a first target nucleic acid (e.g., a first loci) that is targeted by the crRNA(s) in line A, if at least one crRNA is present. The second organism (e.g., line B) would not be modified by the crRNA(s) in the second organism due to lack of a DNA binding domain and/or DNA endonuclease. The method may further comprise crossing the first and second organisms, which may result in modifications in progeny resulting from the cross at a second target nucleic acid (e.g., a second loci) that is unmodified in the first and second organisms, but which may be modified in progeny due to the new combination of unmodified target nucleic acids and the editing machinery. A variety of modifications and/or repair outcomes may be inherited by the progeny of the cross, which may result in allelic diversity that may be phenotypically screened for desirable outcomes. This method may provide a high density of allelic variation that is introduced at a target nucleic acid and may allow for phenotyping to be used as the primary screen.

According to some embodiments of the present invention, a polypeptide, polynucleotide, complex, composition, system, kit, and/or method of the present invention may be used and/or configured to co-modify (e.g., co-edit) genes that confer phenotypes aiding in the isolation of modified plants. This application has high value for crops with low efficiency transformation systems and applies in regard to the requirement for modifying without integration of a transgenic DNA sequence. This may be particularly useful for crops such as cane berries, stonefruits, and other clonally propagated hybrid crops with long generation times. In some embodiments, at least two pegRNAs or a pegRNA and a guide RNA are delivered that are directed to two different target nucleic acids (e.g., two different genes). The first target nucleic acid may be in a trait gene of interest and may be modified using an editing system such as described herein (e.g., such as with a prime editor or any other type of genome editing tool to confer an economically valuable phenotype. The second target nucleic acid may be a different target nucleic acid (e.g., a different gene) that is modified using an editing system (e.g., a prime editor) to confer a phenotype that assists in the identification and/or isolation of cells, tissues, or plants that obtain this edit. For example, the phenotype may be a visual phenotype (e.g., thornless, glossy), a herbicide-resistant phenotype (e.g., ALS inhibitors, glyphosate, PPO inhibitors, etc), and/or an antibiotic-resistant phenotype. A modification conferring such a phenotype may enable the identification and/or isolation of a cell, tissue, or plant that had received the editing machinery. In this way, the provided phenotype acts similar to a selectable marker cassette as a tool to aid in the recovery of modified plants. However, a key difference is that the method does not require the genomic integration of a transgenic marker cassette. Because both pegRNAs may be delivered by the same mechanism, cells that obtain the modification in the second target nucleic acid may have a much greater than random probability of obtaining a modification in the first target nucleic acid. Thus, they may be used to assist in the recovery of non-transgenic, modified organisms (e.g., plants) obtained via transient delivery of the editing tools. A difficulty in trying to obtain non-transgenic, modified plants is the inability to prevent regeneration of untreated cells, requiring handling and screening of thousands or millions of explants. Thus, embodiments of the present invention have major economic benefits and can enable a pipeline of non-transgenic, modified organisms (e.g., plants) that would be impractical to implement without a selection tool.

A target nucleic acid of any plant or plant part may be modified (e.g., mutated, e.g., base edited, cleaved, nicked, etc.) using the nucleic acid constructs of the invention (e.g., SEQ ID NOs: 1-129). Any plant (or groupings of plants, for example, into a genus or higher order classification) may be modified using the nucleic acid constructs of this invention including an angiosperm, a gymnosperm, a monocot, a dicot, a C3, C4, CAM plant, a bryophyte, a fern and/or fern ally, a microalgae, and/or a macroalgae. A plant and/or plant part useful with this invention may be a plant and/or plant part of any plant species/variety/cultivar. The term “plant part,” as used herein, includes but is not limited to, embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell can be in the form of an isolated single cell or can be a cultured cell or can be a part of a higher-organized unit such as, for example, a plant tissue or a plant organ.

Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa,Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, sunflower, raspberry, blackberry, black raspberry and/or cherry.

The present invention further comprises a kit or kits to carry out the methods of this invention. A kit of this invention can comprise reagents, buffers, and apparatus for mixing, measuring, sorting, labeling, etc, as well as instructions and the like as would be appropriate for modifying a target nucleic acid.

In some embodiments, the invention provides a kit comprising one or more nucleic acid constructs of the invention and/or expression cassettes and/or vectors and/or cells comprising the same as described herein, with optional instructions for the use thereof. In some embodiments, a kit may further comprise a CRISPR-Cas guide nucleic acid (corresponding to the CRISPR-Cas nuclease encoded by the polynucleotide of the invention) and/or expression cassette and/or vector comprising the same. In some embodiments, the guide nucleic acid may be provided on the same expression cassette and/or vector as a nucleic acid construct of the invention. In some embodiments, the guide nucleic acid may be provided on a separate expression cassette or vector from that comprising the nucleic acid construct of the invention.

Accordingly, in some embodiments, kits are provided comprising a nucleic acid construct comprising (a) a polynucleotide(s) as provided herein and (b) a promoter that drives expression of the polynucleotide(s) of (a). In some embodiments, the kit may further comprise a nucleic acid construct encoding a guide nucleic acid, wherein the construct comprises a cloning site for cloning of a nucleic acid sequence identical or complementary to a target nucleic acid sequence into backbone of the guide nucleic acid.

In some embodiments, the nucleic acid construct of the invention may be an mRNA that may encode one or more introns within the encoded polynucleotide(s). In some embodiments, a nucleic acid construct of the invention and/or an expression cassette and/or vector comprising the same, may further encode one or more selectable markers useful for identifying transformants (e.g., a nucleic acid encoding an antibiotic resistance gene, herbicide resistance gene, and the like).

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1: Prime Editing Through Recruitment

Previously published strategies for prime editing rely on the use of a reverse transcriptase that is linked to an effector protein through a polypeptide linker. This will naturally restrict the reverse transcriptase to a region accessible by this linker length. To alleviate this issue, methods were developed to recruit reverse transcriptase (RT) to the genomic region, thereby causing a localized concentration increase at the site of editing. Two methods were tested to achieve this goal, recruitment to the Cas effector protein by the addition of peptide epitopes (Suntag), and recruitment to the guide through the addition of hairpin loops.

### Methods:

### Human Cell Testing

Eukaryotic HEK293T (ATCC CRL-3216) cells were cultured in Dulbecco's Modified Eagle's Medium plus GlutaMax (ThermoFisher) supplemented with 10% (v/v) FBS (FBS), at 37° C. with 5% CO2. Cas and reverse transcriptase components were synthesized using solid-state synthesis and subsequently cloned into plasmids behind a CMV promoter. CRISPR RNAs (crRNAs) and pegRNAs (e.g., extended guide nucleic acids) were cloned behind a human U6 promoter. HEK293T cells were seeded on 48-well collagen-coated BioCoat plates (Corning). Cells were transfected at ˜70% confluency. 750 ng of protein plasmid and 250 ng of crRNA expression plasmids were transfected using 1.5 μl of Lipofectamine 3000 (ThermoFisher Scientific) per well according to the manufacturer's protocol. Genomic DNA from transfected cells were obtained after 3 days and indels were detected and quantified using high-throughput Illumina amplicon sequencing.

### Recruitment of RT for Editing Through Epitope Tags

To test the strategy of reverse transcriptase recruitment, a three-plasmid system was designed for expression in human cells. As a control, a PE2 architecture, which consists of a nCas9 protein that is directly fused to the MuLV (5M) (Murine leukemia virus reverse transcriptase with five mutations-D200N+L603W+T330P+T306K+W313F) (Anzalone et al. 2019) reverse transcriptase that is co-delivered with a single pegRNA, was tested alongside the recruitment designs.

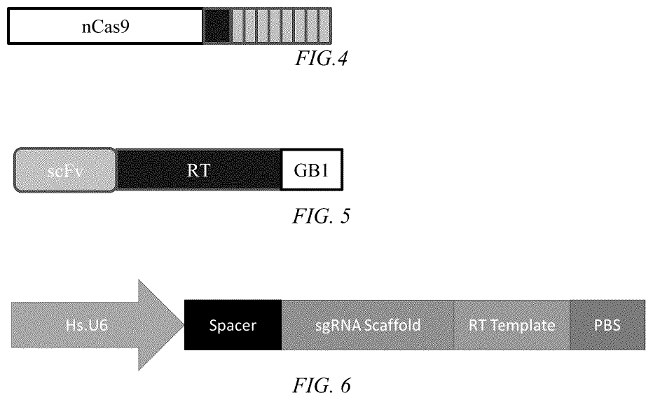

To enable recruitment of the reverse transcriptase to the Cas protein, a set of eight GCN4 epitope motifs was added to the C terminus of a nickase Cas9 protein sequence with a linker between the nCas9 (H840A) and eight GCN4 epitope motifs (nCas9::GCN4) as shown in FIG. 4. The nCas9::GCN4 expression plasmid consists of a CMV promoter driving the nCas9::GCN4 transcription unit, which is separated from an EGFP marker by the P2A ribosomal cleavage sequence. Transcription is terminated by the bGH poly A signal motif. A nucleotide sequence including nCas9::GCN4::P2A: EGFP is provided in SEQ ID NO: 53.

On a separate plasmid the reverse transcriptase is delivered. The reverse transcriptase MuLV-5M is fused to a single-chain variable fragment (scFv) which is an antibody that will bind to the GCN4 epitopes that are fused to nickase Cas9. The reverse transcriptase is followed by the guanine nucleotide-binding protein subunit beta (GB1) sequence for increased solubility (scFV::RT::GB1) as shown in FIG. 5. The scFV::RT::GB1 expression plasmid consists of a CMV promoter driving the scFV::RT::GB1 transcription unit, which is separated from an EGFP marker by the P2A ribosomal cleavage sequence. Transcription is terminated by the bGH poly A signal motif. A nucleotide sequence including scFV::MuLV(5M)::GB1::P2A::EGFP is provided in SEQ ID NO: 54.

Additionally, a third plasmid is delivered that contains the pegRNA (sgRNA scaffold) in the form of a guide scaffold and sequence for Cas9 behind the Homo sapiens U6 promoter (Hs. U6). The sequence for the pegRNA was designed to contain a reverse transcriptase template, and primer binding sequence (PBS) for the purpose of designed edits from the reverse transcriptase, as previously described for prime editing. The pegRNA structure with promoter is shown in FIG. 6. For the purpose of this test, 16 separate guide plasmids were utilized to target 4 separate sites in the human genome as provided in Table 2.

TABLE 2
Guide plasmids for reverse transcriptase recruitment testing.

| Guide | | Designed | |
|---|---|---|---|
| Plasmid | Target | Change | pegRNA Sequence |
| pWISE1580 | FANCF | 5GtoT | GGAATCCCTTCTGCAGCACCGTTTTAGAGCTAG |
| | | | AAATAGCAAGTTAAAATAAGGCTAGTCCGTTA |
| | | | TCAACTTGAAAAAGTGGCACCGAGTCGGTGCG |
| | | | GAAAAGCGATCAAGGTGCTGCAGAAGGGA |
| | | | (SEQ ID NO: 55) |
| pWISE1581 | | 7AtoC | GGAATCCCTTCTGCAGCACCGTTTTAGAGCTAG |
| | | | AAATAGCAAGTTAAAATAAGGCTAGTCCGTTA |
| | | | TCAACTTGAAAAAGTGGCACCGAGTCGGTGCG |
| | | | GAAAAGCGAGCCAGGTGCTGCAGAAGGGAT |
| | | | (SEQ ID NO: 56) |
| pWISE1582 | | 4GATins | GGAATCCCTTCTGCAGCACCGTTTTAGAGCTAG |
| | | | AAATAGCAAGTTAAAATAAGGCTAGTCCGTTA |
| | | | TCAACTTGAAAAAGTGGCACCGAGTCGGTGCG |
| | | | GAAAAGCGATCCAATCGGTGCTGCAGAAGGGA |
| | | | T (SEQ ID NO: 57) |
| pWISE1583 | | 6Gdel | GGAATCCCTTCTGCAGCACCGTTTTAGAGCTAG |
| | | | AAATAGCAAGTTAAAATAAGGCTAGTCCGTTA |
| | | | TCAACTTGAAAAAGTGGCACCGAGTCGGTGCG |
| | | | GAAAAGCGATCAGGTGCTGCAGAAGGGAT |
| | | | (SEQ ID NO: 58) |
| pWISE1584 | RNF2 | 4AtoC | GTCATCTTAGTCATTACCTGGTTTTAGAGCTAG |
| | | | AAATAGCAAGTTAAAATAAGGCTAGTCCGTTA |
| | | | TCAACTTGAAAAAGTGGCACCGAGTCGGTGCA |
| | | | ACGAACACCGCAGGTAATGACTAAGATG |
| | | | (SEQ ID NO: 59) |
| pWISE1585 | | 4AtoG | GTCATCTTAGTCATTACCTGGTTTTAGAGCTAG |
| | | | AAATAGCAAGTTAAAATAAGGCTAGTCCGTTA |
| | | | TCAACTTGAAAAAGTGGCACCGAGTCGGTGCA |
| | | | ACGAACACCCCAGGTAATGACTAAGATG |
| | | | (SEQ ID NO: 60) |
| pWISE1586 | | 5GtoT | GTCATCTTAGTCATTACCTGGTTTTAGAGCTAG |
| | | | AAATAGCAAGTTAAAATAAGGCTAGTCCGTTA |
| | | | TCAACTTGAAAAAGTGGCACCGAGTCGGTGCA |
| | | | ACGAACACATCAGGTAATGACTAAGATG |
| | | | (SEQ ID NO: 61) |
| pWISE1587 | | 6GtoA | GTCATCTTAGTCATTACCTGGTTTTAGAGCTAG |
| | | | AAATAGCAAGTTAAAATAAGGCTAGTCCGTTA |
| | | | TCAACTTGAAAAAGTGGCACCGAGTCGGTGCA |
| | | | ACGAACATCTCAGGTAATGACTAAGATG |
| | | | (SEQ ID NO: 62) |
| pWISE1589 | RUNX1 | 5GtoT | GCATTTTCAGGAGGAAGCGAGTTTTAGAGCTA |
| | | | GAAATAGCAAGTTAAAATAAGGCTAGTCCGTT |
| | | | ATCAACTTGAAAAAGTGGCACCGAGTCGGTGC |
| | | | TGTCTGAAGCAATCGCTTCCTCCTGAAAAT |
| | | | (SEQ ID NO: 63) |
| pWISE1590 | | 6GtoC | GCATTTTCAGGAGGAAGCGAGTTTTAGAGCTA |
| | | | GAAATAGCAAGTTAAAATAAGGCTAGTCCGTT |
| | | | ATCAACTTGAAAAAGTGGCACCGAGTCGGTGC |
| | | | TGTCTGAAGGCATCGCTTCCTCCTGAAAAT |
| | | | (SEQ ID NO: 64) |
| pWISE1591 | | 1ATGins | GCATTTTCAGGAGGAAGCGAGTTTTAGAGCTA |
| | | | GAAATAGCAAGTTAAAATAAGGCTAGTCCGTT |
| | | | ATCAACTTGAAAAAGTGGCACCGAGTCGGTGC |
| | | | TGTCTGAAGCCATCCATGCTTCCTCCTGAAAAT |
| | | | (SEQ ID NO: 65) |
| pWISE1592 | | 2Gdel | GCATTTTCAGGAGGAAGCGAGTTTTAGAGCTA |
| | | | GAAATAGCAAGTTAAAATAAGGCTAGTCCGTT |
| | | | ATCAACTTGAAAAAGTGGCACCGAGTCGGTGC |
| | | | TGTCTGAAGCCATGCTTCCTCCTGAAAAT |
| | | | (SEQ ID NO: 66) |
| pWISE1594 | DMNT1 | 5GtoT | GATTCCTGGTGCCAGAAACAGTTTTAGAGCTA |
| | | | GAAATAGCAAGTTAAAATAAGGCTAGTCCGTT |
| | | | ATCAACTTGAAAAAGTGGCACCGAGTCGGTGC |
| | | | GTCACCACTGTTTCTGGCACCAGG |
| | | | (SEQ ID NO: 67) |
| pWISE1595 | | 6GtoC | GATTCCTGGTGCCAGAAACAGTTTTAGAGCTA |
| | | | GAAATAGCAAGTTAAAATAAGGCTAGTCCGTT |
| | | | ATCAACTTGAAAAAGTGGCACCGAGTCGGTGC |
| | | | GTCACGCCTGTTTCTGGCACCAGG |
| | | | (SEQ ID NO: 68) |
| pWISE1596 | | 1TCAins | GATTCCTGGTGCCAGAAACAGTTTTAGAGCTA |
| | | | GAAATAGCAAGTTAAAATAAGGCTAGTCCGTT |
| | | | ATCAACTTGAAAAAGTGGCACCGAGTCGGTGC |
| | | | TCCCGTCACCCCTGTGATTTCTGGCACCAGG |
| | | | (SEQ ID NO: 69) |
| pWISE1597 | | 3-5AGGdel | GATTCCTGGTGCCAGAAACAGTTTTAGAGCTA |
| | | | GAAATAGCAAGTTAAAATAAGGCTAGTCCGTT |
| | | | ATCAACTTGAAAAAGTGGCACCGAGTCGGTGC |
| | | | TCCCGTCACCGTTTCTGGCACCAGG |
| | | | (SEQ ID NO: 70) |

To examine whether editing could be enabled without recruitment, and thus through overexpression of the reverse transcriptase, an in trans treatment was performed utilizing the same scFV::Reverse Transcriptase::GB1 plasmid, but with a standard nCas9 that did not contain the GCN4 motif.

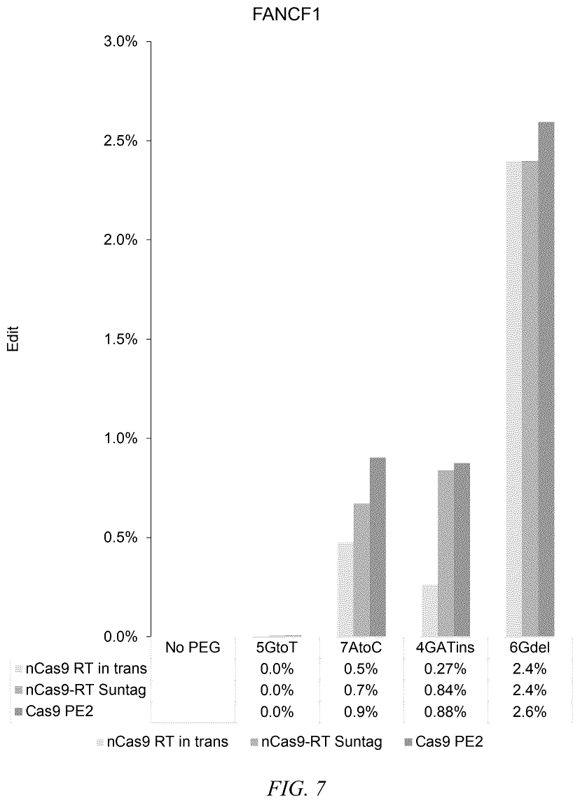

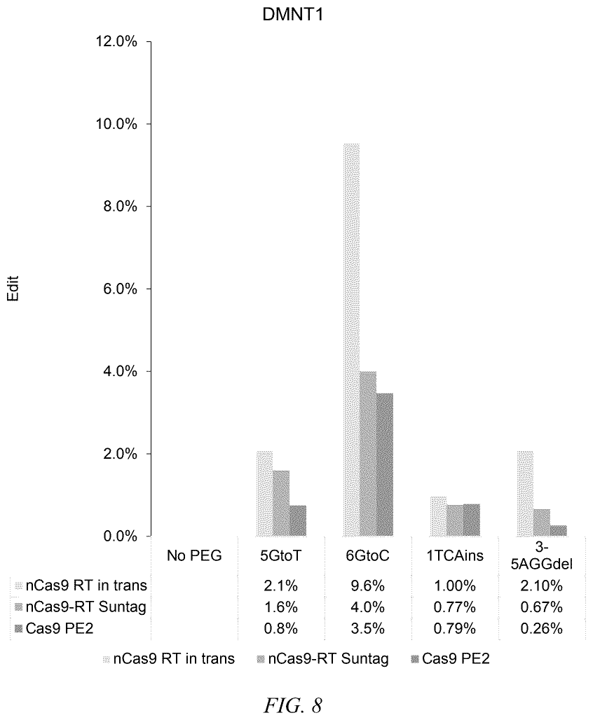

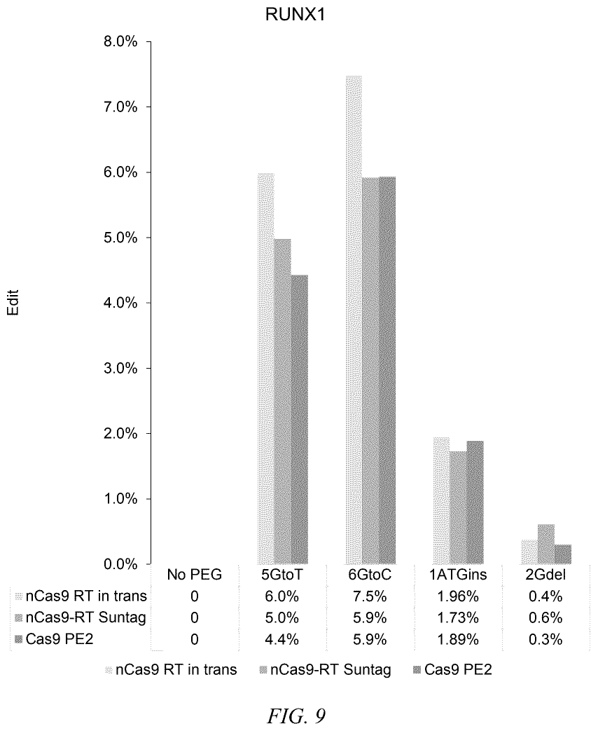

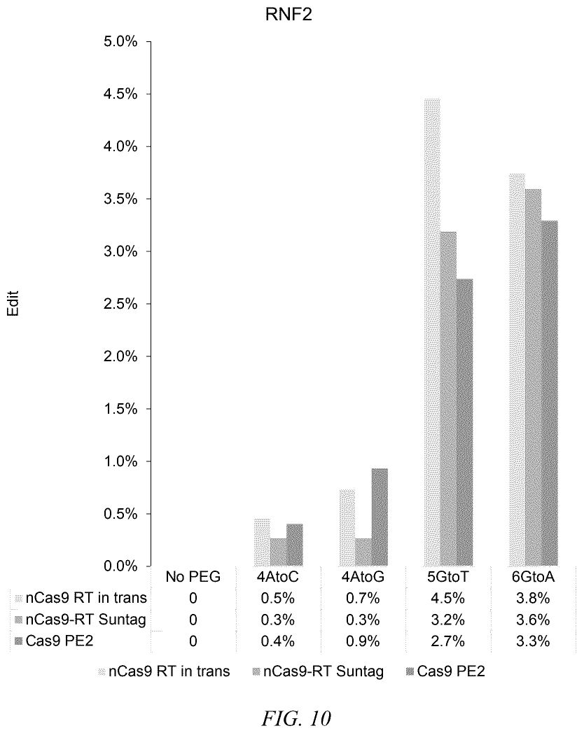

Following delivery to cells, genomic DNA was extracted and the target regions sequenced via amplicon sequencing. As shown in FIGS. 7-10, the recruitment strategy is shown to be equivalent to the previously published PE2 strategy.

### Recruitment of RT to an Upstream Template

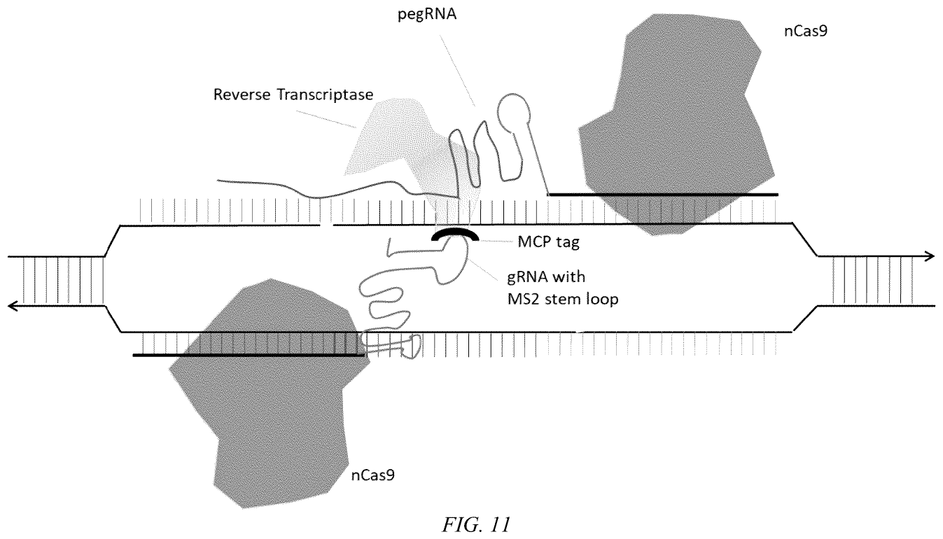

Another method of recruiting the reverse transcriptase to the edit site is to recruit it to the guide itself. To examine this architecture, a strategy was designed wherein one guide would recruit the reverse transcriptase so that it could be positioned nearby the template that is attached to a second guide. As shown in FIG. 11, the recruitment is achieved by utilizing an MS2 RNA stem-loop sequence as a 3′ extension after the sgRNA scaffold. This then recruits the reverse transcriptase which has been engineered to contain the MCP coat protein sequence that will bind to the MS2 loop on the RNA, thus recruiting the reverse transcriptase to the guide RNA. A nucleotide sequence including MCP::MuLV (5M) is provided in SEQ ID NO: 71. The gRNA is positioned to be upstream at a nearby genomic location, the template and primer binding sites designed to be positioned nearby the recruited reverse transcriptase. Nucleotides sequences for the FANCF gRNAs are provided in SEQ ID NOs: 72-73.

The components were separately introduced to human cells on plasmid vectors using the CMV promoter for nCas9 and the reverse transcriptase, and the human U6 promoter for the guide RNAs. A nucleotide sequence including nCas9 (H840A)::P2A::EGFP is provided in SEQ ID NO: 74. As a control, the same edits were attempted with a PE3 strategy, where the reverse transcriptase is linked to the nCas9 and the guide originally containing the MS2 loop is exchanged for a pegRNA containing the template for editing. Nucleotides sequences for the FANCF pegRNAs are provided in SEQ ID NOs: 75-76.

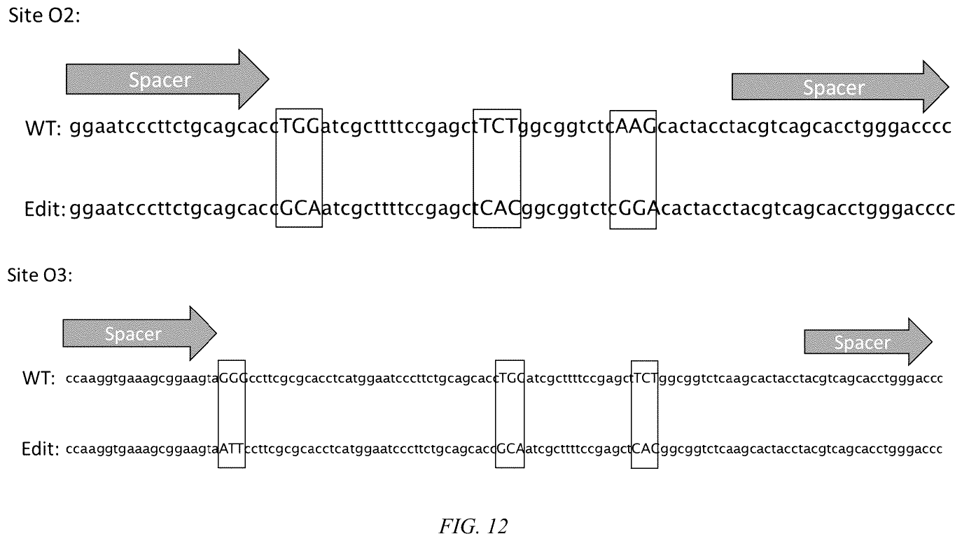

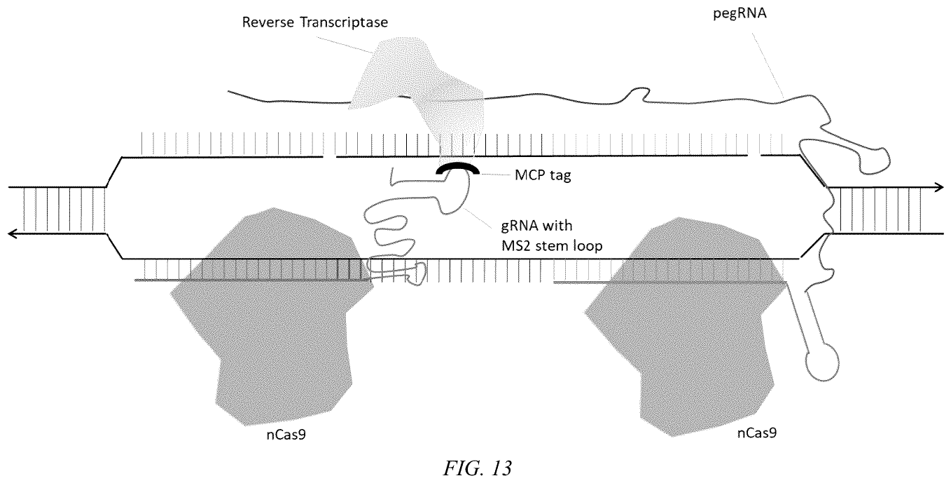

To examine if the recruitment strategy could edit through recruitment two targets at the FANCF locus in human cells were examined that had designed changes between two separate spacers. These targets represent multiple changes, and also a wide window, which is expected to be possible with this strategy. An example of the edits attempted is shown in FIG. 12 for the opposite strand strategy (WT sequence for site O2 (SEQ ID NO: 77); Edit sequence for site O2 (SEQ ID NO: 78); WT sequence for site O3 (SEQ ID NO: 79); and Edit sequence for site O3 (SEQ ID NO: 80). The design is similar for the same-strand strategy as shown in FIG. 13.

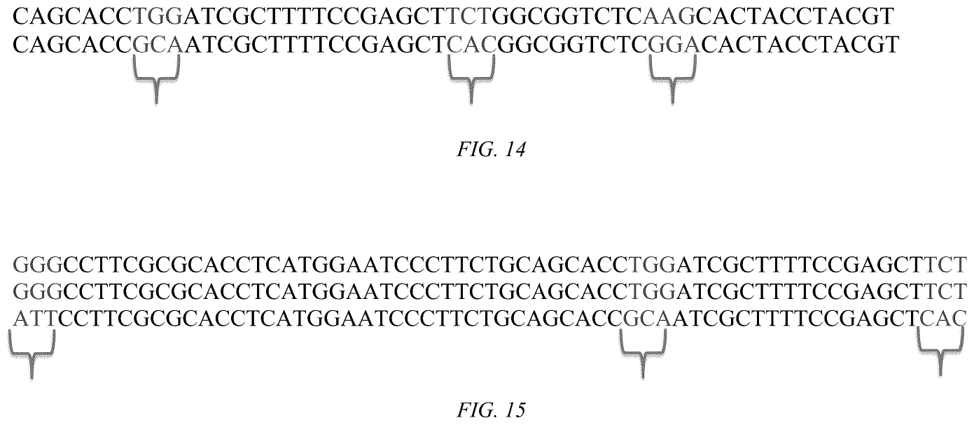

Following delivery of the reagents, the target was sequenced via amplicon sequencing. The PE3 positive control showed editing at both sites for both the opposite and same strand strategies. In the experimental positive edits were only obtained with the opposite strand strategy where editing was observed at both the O2 and O3 sites (FIG. 12), as can be seen in the alignments provided in FIG. 14 (Top line: SEQ ID NO: 81; Bottom line: SEQ ID NO: 82) and FIG. 15 (Top line: SEQ ID NO: 83; Middle line: SEQ ID NO: 84; Bottom line: SEQ ID NO: 85). Edits were not observed when the reverse transcriptase was not introduced to the system.

### Example 2: Evidence of Prime Editing in Plants

### Methods

### Tobacco Infiltration:

Briefly, 4 week old Nicotiana benthamiana plants were used for infiltration with editing constructs. Prior to infiltration, all side shoots and flower buds were removed from plants and plants watered. Constructs were inoculated into LB liquid media with appropriate antibiotics and shaken for 2 days at 28 centigrade. The morning of infiltration, cultures were resuspended in infiltration buffer (10 mM MgCl2, 10 mM MES, pH5.6) and diluted to reach a final OD of 0.7. Prime constructs were mixed at a 3:1 editor to reporter ratio with pWISE711, which contained a ZsGreen fluorescent reporter. Leaves were infiltrated with a needless syringe into the underside of a leaf. Following infiltration, plants were allowed to rest for 1 hour on the lab bench before being moved to a growth chamber. After 5-6 days, plants were collected from growth chamber and treated leaves visualized with a bluelight flashlight. Leaf samples were collected from areas showing fluorescence, and thus presence of introduced constructs. Genomic DNA was collected from these samples before being used for amplicon sequencing.

### Experimental Design:

To adapt the previously published prime editing experiments performed in human cells to plants, an experiment was designed to interrogate different reverse transcriptases and codon optimizations. First, the MuMLV (5M) reverse transcriptase was codon optimized for monocots and dicots. Additionally, the soybean chlorotic mottle virus (SbCMV) (Uniprot ID P15629) and cauliflower mosaic virus (CaMV) (Uniprot ID P03556) reverse transcriptases were optimized for dicots as well as using the native sequence. The various reverse transcriptases used in the experiment are listed in Table 3.

TABLE 3
Reverse Transcriptases Used for Experiment

| | | Codon Optimization |
|---|---|---|
| Name | Reverse Transcriptase | Target |
| MMLV_MO1 (SEQ ID | MuMLV(5M) | Monocot |
| NO: 86) | | |
| MMLV_MO2 (SEQ ID | MuMLV(5M) | Monocot |
| NO: 87) | | |
| MMLV_MO3 (SEQ ID | MuMLV(5M) | Monocot |
| NO: 88) | | |
| MMLV_DO1 (SEQ ID | MuMLV(5M) | Dicot |
| NO: 89) | | |
| MMLV_DO2 (SEQ ID | MuMLV(5M) | Dicot |
| NO: 90) | | |
| SbCMV_Native_Fragment | SbCMV | Native Sequence |
| (SEQ ID NO: 91) | | |
| SbCMV_DO1 (SEQ ID | SbCMV | Dicot |
| NO: 92) | | |
| CaMV_Native_Fragment | CaMV | Native Sequence |
| (SEQ ID NO: 93) | | |
| CaMV_DO1 (SEQ ID | CaMV | Dicot |
| NO: 94) | | |

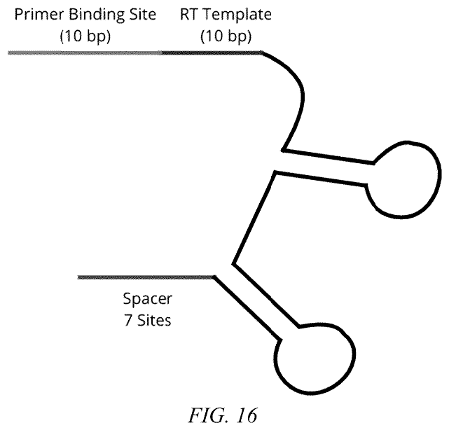

These reverse transcriptases were linked to the nickase variant of SpCas9 (H840A) by the XTEN linker. To examine the impact of expression, each of these editors was placed behind either a double viral promoter consisting of an enhancer from banana streak virus, and promoter and 5′ UTR from dahlia mosaic virus, or ubiquitin 2 promoter from Medicago truncatula. These 18 editor cassettes (9 reverse transcriptase sequences driven by 2 promoters) were then combined with a double guide cassette targeting either the PDS or actin locus of tobacco in the PE3 architecture, containing a pegRNA containing the template for editing with the reverse transcriptase, and a standard sgRNA that will introduce a nick near the target site of the pegRNA; the nicking sequence being one of SEQ ID NOs: 122-128. The pegRNA sequences are provided in Table 4. Each of the pegRNAs have a sequence of one of SEQ ID NOs: 95-101, which was used behind a glycine max 7SL pol III promoter, and included a spacer having a sequence of one of SEQ ID NOs: 102-108, a sgRNA scaffold having a sequence of SEQ ID NO: 129, a primer binding site having a sequence of one of SEQ ID NOs: 115-121, and a reverse transcriptase template having a sequence of one of SEQ ID NOs: 109-114 or SEQ ID NO: 161 that encodes the desired change as shown in FIG. 16. The sgRNA cassette sequences are also provided in Table 4. Each of the sgRNAs have a sequence of one of SEQ ID NOs: 130-136 and included a spacer having a sequence of one of SEQ ID NOs: 137-143 and a sgRNA scaffold having a sequence of SEQ ID NO: 129. For this experiment, all reverse transcriptase templates and primer binding sites were 10 bp in length, and the encoded change was a 6 bp deletion. These 126 constructs were then infiltrated into tobacco leaves as described in the methods section.

TABLE 4
pegRNA and sgRNA sequences.

| | | | Reverse | | |
|---|---|---|---|---|---|
| | | | Transcriptase | Primer Binding | |
| Full Sequence | Spacer | Target | Target | Site | Nicking Spacer |
| AGATGAAACCAAAAGAAGAGGTTTTAGA | AGATGAAACCAAAA | Actin | ATGGCAATGG | TTCTTTTGGTT | CTGGCCCCTCCA |
| GCTAGAAATAGCAAGTTAAAATAAGGCT | GAAGAG | | (SEQ ID | (SEQ ID | TTGTGCAT |
| AGTCCGTTATCAACTTGAAAAAGTGGCA | (SEQ ID NO: 102) | | NO: 109) | NO: 115) | (SEQ ID NO: 122) |
| CCGAGTCGGTGCATGGCAATGGTTCTTTT | | | | | |
| GGTT (SEQ ID NO: 95) | | | | | |
| CACTTCCTATGCACAATGGAGTTTTAGAG | CACTTCCTATGCAC | Actin | TGAGTCTGGC | ATTGTGCATAG | TAATTATCATTA |
| CTAGAAATAGCAAGTTAAAATAAGGCTA | AATGGA | | (SEQ ID | (SEQ ID | TAATTCTT |
| GTCCGTTATCAACTTGAAAAAGTGGCACC | (SEQ ID NO: 103) | | NO: 161) | NO: 116) | (SEQ ID NO: 123) |
| GAGTCGGTGCTGAGTCTGGCATTGTGCAT | | | | | |
| AG (SEQ ID NO: 96) | | | | | |
| TTGGTAGTAGCGACTCCATGGTTTTAGAG | TTGGTAGTAGCGAC | PDS | TTAACTTATG | GGAGTCGCTAC | CATTCAAAACAA |
| CTAGAAATAGCAAGTTAAAATAAGGCTA | TCCATG | | (SEQ ID | (SEQ ID | ACCTTTAA |
| GTCCGTTATCAACTTGAAAAAGTGGCACC | (SEQ ID NO: 104) | | NO: 110) | NO: 117) | (SEQ ID NO: 124) |
| GAGTCGGTGCTTAACTTATGGGAGTCGCT | | | | | |
| AC (SEQ ID NO: 97) | | | | | |
| GCTCTTCCTGCGCCATTAAAGTTTTAGAG | GCTCTTCCTGCGCC | PDS | TAAGTACTTA | AATGGCGCAGG | TTTGCATAATCA |
| CTAGAAATAGCAAGTTAAAATAAGGCTA | ATTAAA | | (SEQ ID | (SEQ ID | ACGCTGAA |
| GTCCGTTATCAACTTGAAAAAGTGGCACC | (SEQ ID NO: 105) | | NO: 111) | NO: 118) | (SEQ ID NO: 125) |
| GAGTCGGTGCTAAGTACTTAAATGGCGC | | | | | |
| AGG (SEQ ID NO: 98) | | | | | |
| GCCGTTAATTTGAGAGTCCAGTTTTAGAG | GCCGTTAATTTGAG | PDS | AGCTGAATTA | ACTCTCAAATT | AATCCTTAACTT |
| CTAGAAATAGCAAGTTAAAATAAGGCTA | AGTCCA | | (SEQ ID | (SEQ ID | ATGCCCCA |
| GTCCGTTATCAACTTGAAAAAGTGGCACC | (SEQ ID NO: 106) | | NO: 112) | NO: 119) | (SEQ ID NO: 126) |
| GAGTCGGTGCAGCTGAATTAACTCTCAA | | | | | |
| ATT (SEQ ID NO: 99) | | | | | |
| GAGATTGTTATTGCTGGTGCGTTTTAGAG | GAGATTGTTATTGCT | PDS | GAAAAAATCA | CAGCAATAACA | ATGAAAACTACA |
| CTAGAAATAGCAAGTTAAAATAAGGCTA | GGTGC | | (SEQ ID | (SEQ ID | AATATAGA |
| GTCCGTTATCAACTTGAAAAAGTGGCACC | (SEQ ID NO: 107) | | NO: 113) | NO: 120) | (SEQ ID NO: 127) |
| GAGTCGGTGCGAAAAAATCACAGCAATA | | | | | |
| ACA (SEQ ID NO: 100) | | | | | |
| GAGGCAAGAGATGTCCTAGGGTTTTAGA | GAGGCAAGAGATGT | PDS | CTTCACCTTT | AGGACATCTCT | GGGAAGGACAC |
| GCTAGAAATAGCAAGTTAAAATAAGGCT | CCTAGG | | (SEQ ID | (SEQ ID) | AAAAGAAAA |
| AGTCCGTTATCAACTTGAAAAAGTGGCA | (SEQ ID NO: 108) | | NO: 114) | NO: 121) | (SEQ ID NO: 128) |
| CCGAGTCGGTGCCTTCACCTTTAGGACAT | | | | | |
| CTCT (SEQ ID NO: 101) | | | | | |
| CTGGCCCCTCCATTGTGCATGTTTTAGAG | CTGGCCCCTCCATT | Actin- | | | |
| CTAGAAATAGCAAGTTAAAATAAGGCTA | GTGCAT | sgRNA | | | |
| GTCCGTTATCAACTTGAAAAAGTGGCACC | (SEQ ID NO: 137) | nicking | | | |
| GAGTCGGTGC (SEQ ID NO: 130) | | guide | | | |
| TAATTATCATTATAATTCTTGTTTTAGAG | TAATTATCATTATAA | Actin- | | | |
| CTAGAAATAGCAAGTTAAAATAAGGCTA | TTCTT | sgRNA | | | |
| GTCCGTTATCAACTTGAAAAAGTGGCACC | (SEQ ID NO: 138) | nicking | | | |
| GAGTCGGTGC (SEQ ID NO: 131) | | guide | | | |
| CATTCAAAACAAACCTTTAAGTTTTAGAG | CATTCAAAACAAAC | PDS- | | | |
| CTAGAAATAGCAAGTTAAAATAAGGCTA | CTTTAA | sgRNA | | | |
| GTCCGTTATCAACTTGAAAAAGTGGCACC | (SEQ ID NO: 139) | nicking | | | |
| GAGTCGGTGC (SEQ ID NO: 132) | | guide | | | |
| TTTGCATAATCAACGCTGAAGTTTTAGAG | TTTGCATAATCAAC | PDS- | | | |
| CTAGAAATAGCAAGTTAAAATAAGGCTA | GCTGAA | sgRNA | | | |
| GTCCGTTATCAACTTGAAAAAGTGGCACC | (SEQ ID NO: 140) | nicking | | | |
| GAGTCGGTGC (SEQ ID NO: 133) | | guide | | | |
| AATCCTTAACTTATGCCCCAGTTTTAGAG | AATCCTTAACTTATG | PDS- | | | |
| CTAGAAATAGCAAGTTAAAATAAGGCTA | CCCCA | sgRNA | | | |
| GTCCGTTATCAACTTGAAAAAGTGGCACC | (SEQ ID NO: 141) | nicking | | | |
| GAGTCGGTGC (SEQ ID NO: 134) | | guide | | | |
| ATGAAAACTACAAATATAGAGTTTTAGA | ATGAAAACTACAAA | PDS- | | | |
| GCTAGAAATAGCAAGTTAAAATAAGGCT | TATAGA | sgRNA | | | |
| AGTCCGTTATCAACTTGAAAAAGTGGCA | (SEQ ID NO: 142) | nicking | | | |
| CCGAGTCGGTGC (SEQ ID NO: 135) | | guide | | | |
| GGGAAGGACACAAAAGAAAAGTTTTAGA | GGGAAGGACACAAA | PDS- | | | |
| GCTAGAAATAGCAAGTTAAAATAAGGCT | AGAAAA | sgRNA | | | |
| AGTCCGTTATCAACTTGAAAAAGTGGCA | (SEQ ID NO: 143) | nicking | | | |
| CCGAGTCGGTGC (SEQ ID NO: 136) | | guide | | | |

### Results

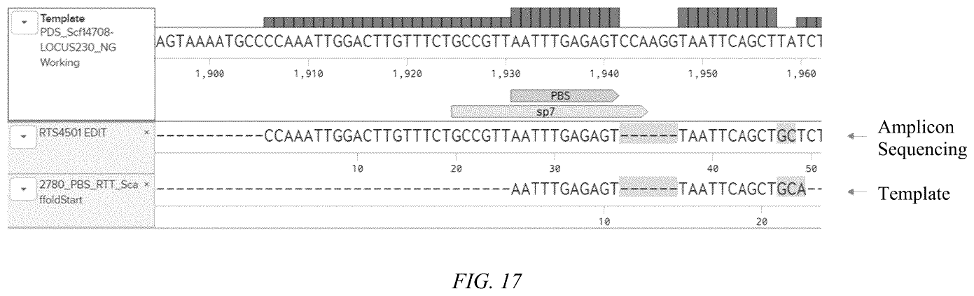

Following amplicon sequencing, positive editing was observed for pWISE2780 (SEQ ID NO: 144) where the MMLV_MO1 codon optimization of the MuMLV (5M) reverse transcriptase was used behind the double viral promoter. The desired 6 bp deletion was observed as well as an insertion of 2 bp that incorporated the start of the scaffold sequence following the reverse transcriptase template, the end result being a 6 bp deletion and a 2 bp insertion as shown in FIG. 17 in which the top row is the targeted gene sequence (SEQ ID NO: 130) with the spacer and primer binding site annotated. The second row (SEQ ID NO: 131) of FIG. 17 is the amplicon sequencing result aligned to the reference showing the targeted deletion and insertion and the bottom row is SEQ ID NO: 132.

The foregoing is illustrative of the present invention, and is not to be construed as limiting thereof. The invention is defined by the following claims, with equivalents of the claims to be included therein.

1-96. (canceled)

97. A method of modifying a target nucleic acid in a plant cell, the method comprising:
contacting the target nucleic acid with:
a Cas9 nickase (nCas9);
a reverse transcriptase, wherein the reverse transcriptase is fused to the nCas9 or recruited to the nCas9; and
an extended guide nucleic acid, thereby modifying the target nucleic acid to provide a modified target nucleic acid in the plant cell.

98. The method of claim 97, wherein the reverse transcriptase is fused to the nCas9.

99. The method of claim 98, wherein the reverse transcriptase is fused to the nCas9 via a peptide linker having a length of 10 to 20 amino acid residues.

100. The method of claim 98, wherein the reverse transcriptase is fused to the C-terminus of the nCas9.

101. The method of claim 97, wherein the reverse transcriptase is recruited to the nCas9.

102. The method of claim 101, wherein the nCas9 is a nCas9 fusion protein comprising the nCas9 fused to a peptide tag and the reverse transcriptase is a reverse transcriptase fusion protein comprising the reverse transcriptase fused to an affinity polypeptide.

103. The method of claim 102, wherein the peptide tag comprises a GCN4 peptide repeat unit and the affinity polypeptide comprises a scFv antibody that is configured to bind the peptide tag.

104. The method of claim 97, further comprising introducing an expression cassette comprising a first polynucleotide encoding a plant specific promoter and a second polynucleotide encoding the nCas9 and/or the reverse transcriptase, wherein the first polynucleotide encoding the plant specific promoter is operably associated with the second polynucleotide encoding the nCas9 and/or the reverse transcriptase.

105. The method of claim 104, wherein the plant specific promoter is a ubiquitin promoter or viral promoter.

106. The method of claim 97, further comprising editing the target nucleic acid to include a mutation and thereby provide the modified target nucleic acid, wherein the mutation is a base deletion, a base insertion, or a base substitution.

107. The method of claim 97, wherein the extended guide nucleic acid is operably linked to a Pol II promoter.

108. The method of claim 97, further comprising contacting the target nucleic acid with a 5′ flap endonuclease (FEN).

109. The method of claim 108, wherein the FEN is a FEN1 polypeptide.

110. The method of claim 108, wherein the FEN is overexpressed in the plant cell.

111. The method of claim 97, wherein the plant cell is a dicot plant cell.

112. The method of claim 97, wherein the plant cell is a monocot plant cell.

113. The method of claim 97, further comprising regenerating the plant cell comprising the modified target nucleic acid to produce a plant comprising the modified target nucleic acid.

114. The method of claim 97, wherein the extended guide nucleic acid comprises a primer binding site and a reverse transcriptase template that is more than 50 nucleotides in length.

115. The method of claim 114, wherein the primer binding site is 1, 2, 3, 4, or 5 to 6, 7, 8, 9, 10, 11, 12, 13, 14, or 15 nucleotides in length.

116. The method of claim 114, wherein the reverse transcriptase template is more than 65 nucleotides in length and is after the primer binding site.
